(19) United States
(12) Patent Application Publication
(10) Pub. No.: US 2008/0058523 A1
(43) Pub. Date: Mar. 6, 2008

(54) Processes for synthesizing piperazine-piperidine compounds

(76) Inventors: Weiguo Liu, Parsippany, NJ (US); Vladimir Dragan, Chester, NY (US); Henry Lee Strong, Somerset, NJ (US); Yanzhong Wu, Bardonia, NY (US); Zhixin Wen, Nanuet, NY (US); Jessica Kangping Liang, Annandale, NJ (US); Haris Durutlic, New Windsor, NY (US); Karen Wiggins Sutherland, New City, NY (US); Anthony Scott Pilcher, Colchester, VT (US)

Correspondence Address: WilmerHale/Wyeth, 60 STATE STREET, BOSTON, MA, 02109, US

(21) Appl. No.: 11/811,328

(22) Filed: Jun. 7, 2007

Related U.S. Application Data
(60) Provisional application No. 60/812,148, filed on Jun. 9, 2006.

(51) Int. Cl. C07D 401/10; C07D 401/14

(52) U.S. Cl. 544/363

(57) ABSTRACT

The present invention relates to processes for synthesizing piperazine-piperidine compounds, and compounds useful as 5-HT_{1A} binding agents, particularly as 5-HT_{1A} receptor antagonists and agonists. The processes also allow for safer and environmentally tolerant production of these useful compounds.

This Application claims the benefit of priority to U.S. Provisional Application No. 60/812,148, filed Jun. 9, 2006, the specification of which is incorporated by reference in its entirety

Throughout this application, various publications are referenced. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure, as it appears in the U.S. Patent and Trademark Office patent file or records, but otherwise reserves any and all copyright rights whatsoever.

### FIELD OF THE INVENTION

The present invention relates to processes and methods for the synthesis of piperazine-piperidine compounds. These processes allow for safer and environmentally tolerant production of these compounds, which are useful as 5-HT_{1A} binding agents, particularly as 5-HT_{1A} receptor antagonists and agonists.

### BACKGROUND OF THE INVENTION

Certain N-aryl-piperazine derivatives possess pharmaceutical activity. In particular, certain N-aryl piperazine derivatives act on the central nervous system (CNS) by binding to 5-HT receptors. In pharmacological testing, it has been shown that the certain N-aryl-piperazine derivatives bind to receptors of the 5-HT_{1A} type. Many of the N-aryl piperazine derivatives exhibit activity as 5-HT_{1A} antagonists. See, for example, W. C. Childers, et al., J. Med. Chem., 48: 3467-3470 (2005), U.S. Pat. Nos. 6,465,482, 6,127,357, 6,469,007, and 6,586,436, and PCT Publication No. WO 97/03982, the disclosures of which are incorporated herein by reference.

Standard processes for the production of piperazine-piperadine derivatives have disadvantages that include hazardous combinations of reaction materials and reaction materials that pose environmental risks. In addition, certain processes utilize chlorinated solvents such as dichloromethane during the production of piperazine-piperadine derivatives. These solvents have undesired toxicity profiles. The processes also produce byproducts that are potentially dangerous to the environment. Finally, the chlorinated compounds used in these processes can produce certain side effects to patients taking pharmaceutical compounds containing residual chlorinated solvents. Accordingly, there remains a need to identify processes that are safer for individuals working with the reaction materials, produce pharmaceutical compounds with decreased toxicity, and generate fewer environmentally toxic byproducts.

### DESCRIPTION OF THE INVENTION

### Definitions

The term “(C₁-C₆)-alkyl” as used herein refers to a linear or branched, saturated hydrocarbon having from 1 to 6 carbon atoms. Representative (C₁-C₆)-alkyl groups include, but are not limited to, methyl, ethyl, propyl, isopropyl, butyl, sec-butyl, tert-butyl, pentyl, isopentyl, neopentyl, hexyl, isohexyl, and neohexyl. In one embodiment, the (C₁-C₆)-alkyl group is substituted with one or more of the following groups: halogen, —N₃, —NO₂, —CN, —OR′, —SR′, —SO₂R′, —SO₂N(R′)₂, —N(R′)₂, —COR′, —CO₂R′, —NR′CO₂R′, —NR′COR′, —NR′CONR′, or —CON(R′)₂, wherein each R′ is independently hydrogen or unsubstituted (C₁-C₆)-alkyl.

The term “(C₂-C₆)-alkenyl” as used herein refers to a linear or branched hydrocarbon having from 2 to 6 carbon atoms and having at least one carbon-carbon double bond. In one embodiment, the (C₂-C₆)-alkenyl has one or two double bonds. The (C₂-C₆)-alkenyl moiety may exist in the E or Z conformation and the compounds of the present invention include both conformations. In one embodiment, the (C₂-C₆)-alkenyl group is substituted with one or more of the following groups: halogen, —N₃, —NO₂, —CN, —OR′, —SR′, —SO₂R′, —SO₂N(R′)₂, —N(R′)₂, —COR′, —CO₂R′, —NR′CO₂R′, —NR′COR′, —NR′CONR′, or —CON(R′)₂, wherein each R′ is independently hydrogen or unsubstituted (C₁-C₆)-alkyl.

The term “(C₂-C₆)-alkynyl” as used herein refers to a linear or branched hydrocarbon having from 2 to 6 carbon atoms and having at least one carbon-carbon triple bond. In one embodiment, the (C₂-C₆)-alkenyl group is substituted with one or more of the following groups: halogen, —N₃, —NO₂, —CN, —OR′, —SR′, —SO₂R′, —SO₂N(R′)₂, —N(R′)₂, —COR′, —CO₂R′, —NR′CO₂R′, —NR′COR′, —NR′CONR′, or —CON(R′)₂, wherein each R′ is independently hydrogen or unsubstituted (C₁-C₆)-alkyl.

“(C₁-C₆)-haloalkyl” refers to a C₁-C₆ alkyl group, as defined above, wherein one or more of the C₁-C₆ alkyl group's hydrogen atoms has been replaced with —F, —Cl, —Br or —I. Representative examples of an alkylhalo group include, but are not limited to, —CH₂F, —CCl₃, —CF₃, —CH₂Cl, —CH₂CH₂Br, —CH₂CH₂I, —CH₂CH₂CH₂F, —CH₂CH₂CH₂Cl, —CH₂CH₂CH₂CH₂Br, —CH₂CH₂CH₂CH₂I, —CH₂CH₂CH₂CH₂CH₂Br, —CH₂CH₂CH₂CH₂CH₂I, —CH₂CH(Br)CH₃, —CH₂CH(Cl)CH₂CH₃, —CH(F)CH₂CH₃, —C(CH₃)₂(CH₂Cl), —CH₂CH₂CH₂CH₂CH₂CH₂Br, and —CH₂CH₂CH₂CH₂CH₂CH₂I.

The term “(C₁-C₆)-alkyoxy,” as used herein, means a functional group having the formula L-O in which L is a linear or branched, saturated hydrocarbon having from 1 to 6 carbon atoms. Representative (C₁-C₆)-alkyoxy groups include, but are not limited to, methoxy, ethoxy, propoxy, isopropoxy, butoxy, sec-butoxy, tert-butoxy, pentoxy, isopentyloxy, neopentoxy, hexyloxy, isohexyloxy, and neohexyloxy. In one embodiment, the (C₁-C₆)-alkyl group is substituted with one or more of the following groups: halogen, —N₃, —NO₂, —CN, —OR′, —SR′, —SO₂R′, —SO₂N(R′)₂, —N(R′)₂, —COR′, —CO₂R′, —NR′CO₂R′, —NR′COR′, —NR′CONR′, or —CON(R′)₂, wherein each R′ is independently hydrogen or unsubstituted (C₁-C₆)-alkyl.

The term “aryl” as used herein refers to an aromatic species containing 1 to 3 aromatic rings, either fused or linked. In one embodiment, the aryl group is substituted with one or more of the following groups: C₁-C₆)-alkyl, —V-halogen, —V—N₃, —V—NO₂, —V—CN, —V—OR′, —V—SR′, —V—SO₂R′, —V—SO₂N(R′)₂, —V—N(R′)₂, —V—COR′, —V—CO₂R′, —V—NR′CO₂R′, —V—NR′COR′, —V—NR′CONR′, or —V—CON(R′)₂, wherein each R′ is independently hydrogen or unsubstituted (C₁-C₆)-alkyl; and wherein each V is independently a bond or (C₁-C₆)-alkyl.

The term “conditions effective to” as used herein refers to synthetic reaction conditions which will be apparent to those skilled in the art of synthetic organic chemistry.

The term “cyclic group” as used herein includes a cycloalkyl group and a heterocyclic group. Any suitable ring position of the cyclic group may be covalently linked to the defined chemical structure. In one embodiment, the cyclic group is substituted with one or more of the following groups: C₁-C₆)-alkyl, —V-halogen, —V—N₃, —V—NO₂, —V—CN, —V—OR′, —V—SR′, —V—SO₂R′, —V—SO₂N(R′)₂, —V—N(R′)₂, —V—COR′, —V—CO₂R′, —V—NR′CO₂R′, —V—NR′COR′, —V—NR′CONR′, or —V—CON(R′)₂, wherein each R′ is independently hydrogen or unsubstituted (C₁-C₆)-alkyl; and wherein each V is independently a bond or (C₁-C₆)-alkyl.

The term “cycloalkyl group” as used herein refers to a three- to seven-membered saturated or partially unsaturated carbon ring. Any suitable ring position of the cycloalkyl group may be covalently linked to the defined chemical structure. Exemplary cycloalkyl groups include cyclopropyl, cyclobutyl, cyclopentyl, cyclohexyl, and cycloheptyl. In one embodiment, the cycloalkyl group is substituted with one or more of the following groups: C₁-C₆)-alkyl, —V-halogen, —V—N₃, —V—NO₂, —V—CN, —V—OR′, —V—SR′, —V—SO₂R′, —V—SO₂N(R′)₂, —V—N(R′)₂, —V—COR′, —V—CO₂R′, —V—NR′CO₂R′, —V—NR′COR′, —V—NR′CONR′, or —V—CON(R′)₂, wherein each R′ is independently hydrogen or unsubstituted (C₁-C₆)-alkyl; and wherein each V is independently a bond or (C₁-C₆)-alkyl.

The term “halogen” as used herein refers to fluorine, chlorine, bromine, and iodine.

The term “heterocyclic group” as used herein refers to a monocyclic, bicyclic or tricyclic, saturated, partially saturated, or unsaturated cycloalkyl group in which one to four of the ring carbon atoms have been independently replaced with a N, O, or S atom and the ring or each ring is three to seven-membered. Any suitable ring position of the heterocyclic group may be covalently linked to the defined chemical structure. Exemplary heterocyclic groups include, but are not limited to, azepanyl, azetidinyl, aziridinyl, furanyl, furazanyl, homopiperazinyl, imidazolidinyl, imidazolinyl, isothiazolyl, isoxazolyl, morpholinyl, oxadiazolyl, oxazolidinyl, oxazolyl, oxazolidinyl, pyrimidinyl, phenanthridinyl, phenanthrolinyl, piperazinyl, piperidinyl, pyranyl, pyrazinyl, pyrazolidinyl, pyrazolinyl, pyrazolyl, pyridazinyl, pyridooxazolyl, pyridoimidazolyl, pyridothiazolyl, pyridinyl, pyrimidinyl, pyrrolidinyl, pyrrolinyl, quinuclidinyl, tetrahydrofuranyl, thiadiazinyl, thiadiazolyl, thienyl, thienothiazolyl, thienooxazolyl, thienoimidazolyl, thiomorpholinyl, thiophenyl, triazinyl, and triazolyl. In one embodiment, the heterocyclic group is substituted with one or more of the following groups: C₁-C₆)-alkyl, —V-halogen, —V—N₃, —V—NO₂, —V—CN, —V—OR′, —V—SR′, —V—SO₂R′, —V—SO₂N(R′)₂, —V—N(R′)₂, —V—COR′, —V—CO₂R′, —V—NR′CO₂R′, —V—NR′COR′, —V—NR′CONR′, or —V—CON(R′)₂, wherein each R′ is independently hydrogen or unsubstituted (C₁-C₆)-alkyl; and wherein each V is independently a bond or (C₁-C₆)-alkyl.

The term “isolated and purified” as used herein refers to separate from other components of a reaction mixture or a natural source. In certain embodiments, the isolate contains at least about 50%, at least about 55%, at least about 60%, at least about 65%, at least about 70%, at least about 75%, at least about 80%, at least about 85%, at least about 90%, at least about 95%, or at least about 98% of the compound or pharmaceutically acceptable salt of the compound by weight of the isolate.

The term “pharmaceutically acceptable salt” as used herein refers to a salt of an acid and one or more basic nitrogen atoms of a compound of the present invention. Exemplary salts include, but are not limited to, sulfate, citrate, acetate, oxalate, chloride, hydrochloride, bromide, hydrobromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, succinate, fumarate, maleate, malonate, mandelate, malate, phthalate, and pamoate. The term “pharmaceutically acceptable salt” as used herein also refers to a salt of a compound of the present invention having an acidic functional group, such as a carboxylic acid functional group, and a base. Exemplary bases include, but are not limited to, hydroxide of alkali metals including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like. The term “pharmaceutically acceptable salt” also includes a hydrate of a compound of the present invention.

The term “phenyl” as used herein refers to a substituted or unsubstituted phenyl group. In one embodiment, the phenyl group is substituted with one or more of the following groups: —V-halogen, —V—N₃, —V—NO₂, —V—CN, —V—OR′, —V—SR′, —V—SO₂R′, —V—SO₂N(R′)₂, —V—N(R′)₂, —V—COR′, —V—CO₂R′, —V—NR′CO₂R′, —V—NR′COR′, —V—NR′CONR′, or —V—CON(R′)₂, wherein each R′ is independently hydrogen or unsubstituted (C₁-C₆)-alkyl; and wherein each V is independently a bond or (C₁-C₆)-alkyl.

The term “substantially free of its corresponding opposite enantiomer” as used herein means that the compound contains no more than about 10% by weight of its corresponding opposite enantiomer. In other embodiments, the compound that is substantially free of its corresponding opposite entantiomer contains no more than about 5%, no more than about 1%, no more than about 0.5%, or no more than about 0.1% by weight of its corresponding opposite enantiomer. An enantiomer that is substantially free of its corresponding opposite enantiomer includes a compound that has been isolated and purified or has been prepared substantially free of its corresponding opposite enantiomer.

The term “5-HT_{1A}-related disorder” as used herein refers to a condition which is mediated through the 5-HT_{1A} receptor. In some embodiments, a 5-HT_{1A}-related disorder is a condition for which it would be beneficial to prevent activation of the 5-HT_{1A} receptor. In other embodiments, a 5-HT_{1A}-related disorder is a condition for which it would be beneficial to activate the 5-HT_{1A} receptor. In one embodiment, a 5-HT_{1A}-related disorder affects the central nervous system (i.e., a CNS-related disorder). Exemplary 5-HT_{1A}-related disorders include, without limitation, depression, single episodic or recurrent major depressive disorders, dysthymic disorders, depressive neurosis and neurotic depression, melancholic depression including anorexia, weight loss, insomnia, early morning waking or psychomotor retardation; atypical depression (or reactive depression) including increased appetite, hypersomnia, psychomotor agitation or irritability, seasonal affective disorder, pediatric depression, child abuse induced depression and postpartum depression; bipolar disorders or manic depression, for example, bipolar I disorder, bipolar II disorder and cyclothymic disorder; conduct disorder; disruptive behavior disorder; disorders of attention and learning such as attention deficit hyperactivity disorder (ADHD) and dyslexia; behavioral disturbances associated with mental retardation, autistic disorder, pervasive development disorder and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia, substance-induced psychotic disorder, shared psychotic disorder, and psychotic disorder due to a general medical condition; delirium, dementia, and amnestic and other cognitive or neurodegenerative disorders, such as Parkinson's disease (PD), Huntington's disease (HD), Alzheimer's disease, senile dementia, dementia of the Alzheimer's type, mild cognitive impairment (MCI), memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies; cognitive deficits associated with neurological conditions including, for example, Parkinson's disease (PD), Huntington's disease (HD), Alzheimer's disease; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremor; chemical dependencies and addictions (e.g., dependencies on, or addictions to, alcohol, heroin, cocaine, benzodiazepines, nicotine, or phenobarbital); behavioral addictions such as an addiction to gambling; and ocular disorders such as glaucoma and ischemic retinopathy; and sexual dysfunction when used in combination with an SSRI, sexual dysfunction associated with drug treatment (e.g., treatment with SSRI's).

Methods for Making the Compounds of the Invention

The methods of the present invention can be utilized to generate piperazine-piperidine derivatives and pharmaceutically acceptable salts thereof. The present invention provides methods used in the synthesis of compounds of Formula (V):

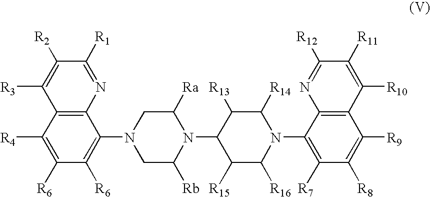

and pharmaceutically acceptable salts and hydrates thereof,

wherein R₁, R₂, R₃, R₄, R₅, R₆, R₇, R₈, R₉, R₁₀, R₁₁, R₁₂, R₁₃, R₁₄, R₁₅, and R₁₆, are each independently —H, (C₁-C₆)-alkyl, (C₁-C₆)-haloalkyl, (C₂-C₆)-alkenyl, or (C₂-C₆)-alkynyl, halogen, —CF₃, —NO₂, —CN, —OR₂₅, —OSO₂R₂₅, —SR₂₅, —SO₂R₂₅, —SO₂N(R₂₅)₂, N(R₂₅)₂, C(O), —COR₂₅, —CO₂R₂₅, —NR₂₅CO₂R₂₅, —NR₂₅COR₂₅, —NR₂₅CON(R₂₅)₂, or —CON(R₂₅)₂;

Rₐ and R_{b} are each independently —H or —CH₃; and

R₂₅ is —H; or linear or branched (C₁-C₆)-alkyl, (C₁-C₆)-haloalkyl, (C₂-C₆)-alkenyl, or (C₂-C₆)-alkynyl.

The present invention provides methods for the synthesis of compounds of Formula (Va):

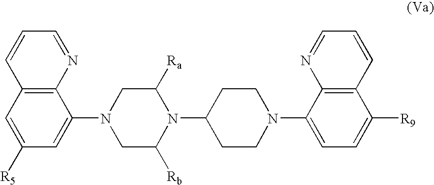

and pharmaceutically acceptable salts and hydrates thereof,

wherein R₅ and R₉ are each independently —H, (C₁-C₆)-alkyl, (C₁-C₆)-haloalkyl, (C₂-C₆)-alkenyl, or (C₂-C₆)-alkynyl, halogen, —CF₃, —NO₂, —CN, —OR₂₅, —OSO₂R₂₅, —SR₂₅, —SO₂R₂₅, —SO₂N(R₂₅)₂, —N(R₂₅)₂, C(O), —COR₂₅, —CO₂R₂₅, —NR₂₅CO₂R₂₅, —NR₂₅COR₂₅, —NR₂₅CON(R₂₅)₂, or —CON(R₂₅)₂;

Rₐ and R_{b} are each independently —H or —CH₃; and

R₂₅ is —H; or linear or branched (C₁-C₆)-alkyl, (C₁-C₆)-haloalkyl, (C₂-C₆)-alkenyl, or (C₂-C₆)-alkynyl.

In some embodiments, the compound of Formula (Va) is optionally substituted such that R₅ and R₉ are each independently hydrogen, halogen, (C₁-C₆)-alkyl, (C₁-C₆)-haloalkyl, (C₂-C₆)-alkenyl, or (C₂-C₆)-alkynyl, —CF₃, —NO₂, —CN, or —OR₂₅. In other embodiments, R₅ is hydrogen or —OR₂₅ such that R₂₅ is (C₁-C₆)-alkyl, and R₉ is a halogen such as fluorine, chlorine, or bromine. In more specific embodiments, the process of the present invention is used to synthesize a compound of Formula (Vb):

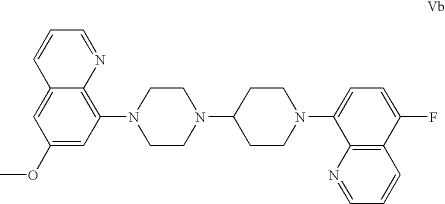

Generally, the methods of the present invention allow the production of compounds of Formulas (V), (Va), and (Vb) with increased safety during production and decreased toxicity after production of the compound. The present invention provides methods by which compounds of Formula (V), (Va), and (Vb) are synthesized by processes utilizing less volatile reaction steps. For instance, the processes of the present inventions do not utilize m-nitrobenzene compounds at high temperatures, which can create potentially volatile reactions leading to significant safety concerns.

Furthermore, the addition of nitrobenzenes or other nitro-containing compounds is carried out in a series of steps to reduce the potential for volatile exothermic reactions. In some embodiments, this is accomplished by premixing nitro-containing compounds such as 4-nitrobenzene with optionally substituted phenyl intermediates at room temperature prior to the addition of such a mixture to hot sulfuric acid. In certain embodiments, the addition of the mixture containing the optionally substituted intermediate and the nitro-containing compound is added slowly to the hot sulfuric acid, thereby allowing the nitro-containing compounds to be consumed during the addition and to reduce the amount of nitro-containing compounds in the hot sulfuric acid.

The present invention further reduces the reliance of the process on the use of chlorinated solvents such as dichloroethane during the production of the di-quinoline compounds of Formula (V). The reduction in the use of certain chlorinated compounds improves the toxicity profile of the resulting pharmaceutical compounds and decreases the potential for environmentally hazardous byproducts produced during the production.

In further embodiments, organic solvents such as toluene are utilized to produce compounds of Formulas (V), (Va), and (Vb). The use of toluene as opposed to chlorinated compounds allows for the production of pharmaceutical compounds having reduced levels of chlorinated byproducts in the final pharmaceutical product.

In certain embodiments, dichloromethane is used rather than dichloroethane to reduce the potential pharmaceutical toxicity of the solvents used during production of the piperazine-piperidine compounds. The Food and Drug Administration (“FDA”) has classified dichloromethane as a class 2 compound, whereas dichloroethane is a class 1 compound. FDA guidelines for pharmaceutical manufacturing state that Class 1 solvents should be avoided because of their unacceptable toxicity or their deleterious environmental effect. (See ICH Guideline Q3C Impurities: Residual Solvents.) In situations where Class 1 solvents must be used, their concentration is generally limited to less than 1500 ppm, with most solvents in this group being limited to less than 10 ppm (See id.). In particular, dichloroethane levels must be limited to 5 ppm (See id.). In contrast, the guidelines state that dichloromethane may be present in concentrations up to 600 ppm (See id.). Accordingly, by improving the synthesis of the compounds of Formulas (V), (Va), and (Vb) by replacing dichloroethane with dichloromethane the toxicity profile of the resulting compounds is reduced and the environmental impact decreased.

It should also be noted that the processes of the present invention allow for more cost effective production of piperazine-piperidine compounds. In some embodiments, the use of the cheaper intermediate bis(2-chloroethyl)amine hydrochloride to form the piperazine component of the compounds of the present invention creates a more cost effective synthesis.

In some embodiments, the toxicity profile of the present invention is improved by eliminating the use of the highly toxic sodium cyanoborohydride compound in the reductive amination step. The use of sodium cyanoborohydride represents a significantly dangerous compound that requires must be removed completely from the synthesized pharmaceutical compounds. Therefore, the present invention provides processes that do not use this compound and improves the safety connected with the production and use of the di-quinoline compounds.

In alternative embodiments, quinoline substituted piperazine intermediates are prepared by way of an intermediate of Formula VIII:

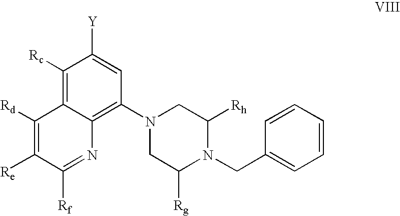

wherein Y, R_{c}, R_{d}, Rₑ, R_{f}, are each independently hydrogen, (C₁-C₆)-alkyl, (C₁-C₆)-haloalkyl, (C₂-C₆)-alkenyl, or (C₂-C₆)-alkynyl, halogen, —CF₃, —NO₂, —CN, —OR₂₅, —OSO₂R₂₅, —SR₂₅, —SO₂R₂₅, —SO₂N(R₂₅)₂, —N(R₂₅)₂, C(O), —COR₂₅, —CO₂R₂₅, —NR₂₅CO₂R₂₅, —NR₂₅COR₂₅, —NR₂₅CON(R₂₅)₂, or —CON(R₂₅)₂; and

R₂₅ is —H; or linear or branched (C₁-C₆)-alkyl, (C₁-C₆)-haloalkyl, (C₂-C₆)-alkenyl, or (C₂-C₆)-alkynyl; and R_{g} and Rₕ are each independently —H or CH₃.

In some embodiments, Y is hydrogen, (C₁-C₆)-alkyl, (C₁-C₆)-haloalkyl, (C₂-C₆)-alkenyl, or (C₂-C₆)-alkynyl, and R₂₅ is —H; or linear or branched (C₁-C₆)-alkyl, (C₁-C₆)-haloalkyl, (C₂-C₆)-alkenyl, or (C₂-C₆)-alkynyl.

In other embodiments, R_{c}, R_{d}, Rₑ, R_{f}, are each independently hydrogen, (C₁-C₆)-alkyl, (C₁-C₆)-haloalkyl, (C₂-C₆)-alkenyl, or (C₂-C₆)-alkynyl, halogen, —CF₃, —NO₂, and —CN.

In certain embodiments, R_{c}, R_{d}, Rₑ, R_{f}, are each independently H.

In still other embodiments, Y is methoxy, and R_{c}, R_{d}, Rₑ, R_{f}, are each independently H.

In the alternative embodiments, the synthesis of compounds of Formula Ia, described below, goes through a step of removal of the benzene group by hydrogen transfer in the presence of 1-methylcyclohexene, which decreases the environmental impact of synthesizing piperazine-piperidine compounds. This is shown in the following Scheme 1:

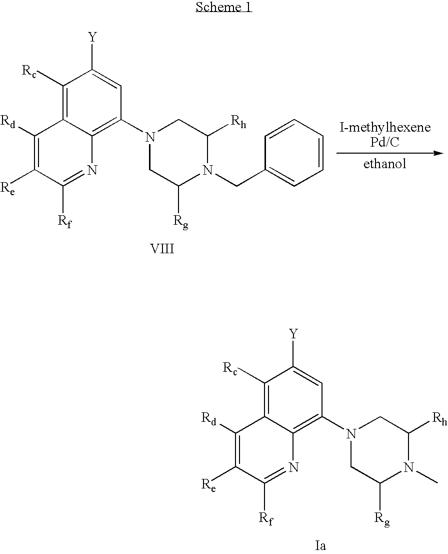

wherein R_{c}, R_{d}, Rₑ, R_{f}, are each independently hydrogen, (C₁-C₆)-alkyl, (C₁-C₆)-haloalkyl, (C₂-C₆)-alkenyl, or (C₂-C₆)-alkynyl, halogen, —CF₃, —NO₂, —CN, —OR₂₅, —OSO₂R₂₅, —SR₂₅, —SO₂R₂₅, —SO₂N(R₂₅)₂, —N(R₂₅)₂, C(O), —COR₂₅, —CO₂R₂₅, —NR₂₅CO₂R₂₅, —NR₂₅COR₂₅, —NR₂₅CON(R₂₅)₂, or —CON(R₂₅)₂; and

R₂₅ is —H; or linear or branched (C₁-C₆)-alkyl, (C₁-C₆)-haloalkyl, (C₂-C₆)-alkenyl, or (C₂-C₆)-alkynyl; and R_{g} and Rₕ, are each independently —H or CH₃.

The reduction in environmental hazard is due to 1-methylhexene yielding the less toxic byproduct toluene rather than benzene. Accordingly, the processes of the present invention provide for environmentally less hazardous byproducts, requiring less stringent disposal and containment of such byproducts.

The methods of the present invention also allow for the synthesis of the compounds of Formula (V), (Va), and (Vb) by way of quinoline-substituted piperazine compounds. The present invention provides processes by which quinoline-substituted piperazine compounds are isolated from a highly viscous state. In certain embodiments, the optionally substituted quinoline substituted piperadines are isolated by first introducing a dicarboxylic acid in conditions effective at yielding an acid addition salt of the quinoline-substituted piperazine. In one embodiment, the dicarboxylic acid is a (C₃-C₁₂)-alkyl dicarboxylic acid, i.e. malonic acid or a homologue thereof. In one embodiment, the dicarboxylic acid is a straight chain alkyl dicarboxylic acid. In one embodiment, the dicarboxylic acid is adipic acid, yielding the adipate salt of the optionally substituted quinoline-substituted piperazine.

In some embodiments, the optionally substituted quinoline substituted piperadines are 6-methoxy-8-(1-piperazinyl)quinoline.

In certain embodiments, the salt is further reacted in the presence of a base and organic solvent in conditions effective at yielding a solution containing the isolated quinoline substituted piperazine. Accordingly, the process of the present invention allows for effective isolation of quinoline-substituted piperazine compounds, even from viscous solutions not normally amendable to processing of the compounds.

The compounds and pharmaceutically acceptable salts of compounds can be prepared using a variety of methods of the present invention starting from commercially available compounds, known compounds, or compounds prepared by known methods. General synthetic routes to many of the compounds of the invention are included in the following schemes. The methods for making some intermediates of the invention are described in PCT Publication No. WO04/024731 and U.S. Pat. No. 4,465,482, both of which are hereby incorporated by reference. It is understood by those skilled in the art that protection and deprotection steps not shown in the Schemes may be required for these syntheses, and that the order of steps may be changed to accommodate functionality in the target molecule.

It should also be noted that a variety of intermediates can also be used to produce the compounds of Formula (V), (VIa), and (VIb). For instance, the quinoline-substituted compound of Formula Ib is used to produce a portion of the compound of Formula (V), (Va), and (Vb):

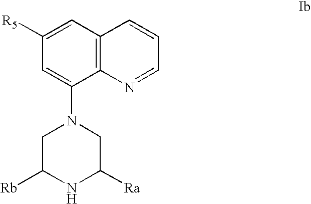

The intermediate of Formula Ib can be optionally substituted. In some embodiments, R₅ is hydrogen, (C₁-C₆)-alkyl, (C₁-C₆) haloalkyl, (C₂-C₆)-alkenyl, or (C₂-C₆)allynyl, CF₃, OR₂₅, —OSO₂R₂₅, —SR₂₅, —SO₂R₂₅, —SO₂N(R₂₅)₂, N(R₂₅)₂, C(O), COR₂₅, CO₂R₂₅, NR₂₅CO₂R₂₅, NR₂₅COR₂₅, —NR₂₅CON(R₂₅)₂, or CON(R₂₅)₂, and R₂₅ is —H; or linear or branched (C₁-C₆)-alkyl, (C₁-C₆) haloalkyl, (C₂-C₆)-alkenyl, or (C₂-C₆)-alkynyl. In some embodiments, R₅ is hydrogen, (C₁-C₆)-alkyl, halogen, —CF₃, or —OR₂₅. In other embodiments, R₅ is OR₂₅ and R₂₅ is —H; or linear or branched (C₁-C₆)-alkyl, (C₁-C₆) haloalkyl, (C₂-C₆)-alkenyl, or (C₂-C₆)-alkynyl. In certain embodiments, R₅ is a methoxy. In the above embodiments, Rₐ and R_{b} are each independently hydrogen or methyl. In some embodiments, R₅ is methoxy and Rₐ and R_{b} are hydrogen, which yields a compound of Formula Ic:

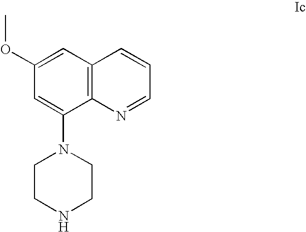

In addition, various intermediates can be utilized in the processes of the present invention to produce the optionally substituted quinolines that serve as intermediates of the quinoline-substituted piperidine components of the compounds of Formulas (V), (Va), and (Vb). In some embodiments, the compound of Formula IIa is used:

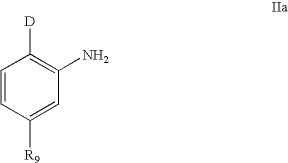

wherein R₉ is hydrogen, (C₁-C₆)-alkyl, halogen, —CF₃, or —OR₂₅; R₉ is any halogen; and D is any halogen.

In some embodiments, R₉ is any halogen and D is any halogen. In other embodiments, R₉ is fluorine and D is chlorine or bromine. In certain embodiments, R₉ is fluorine and D is bromine to yield the structure of Formula IIb:

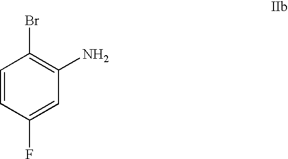

The above Formula IIb can be used to produce the quinoline structure that is used to synthesize the compounds of Formulas (V), (Va), and (Vb). In some embodiments, the optionally substituted quinoline compounds used in the processes of the present inventions have the structure of Formula IIIa:

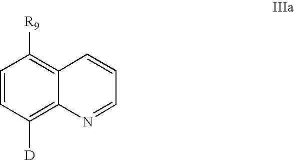

The compound of Formula IIIa are optionally substituted such that R₉ is hydrogen or any halogen and D is a good leaving group. In one embodiment, D is halogen. In some embodiments, R₉ is fluorine and D is bromine or chlorine. In other embodiments, R₉ is fluorine and D is bromine to yield the structure of Formula IIIb:

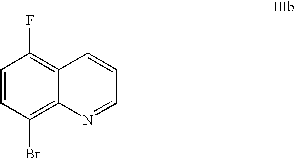

To further describe the methods and processes of the invention, the following non-limiting schemes illustrate the various synthetic means that can be utilized to produce pharmaceutically advantageous piperazine-piperidine compounds.

For instance, Scheme 1 illustrates the production of compounds of Formula (I). As shown in Scheme 1, a compound of Formula I and a compound of Formula IVa are reacted under conditions effective to produce the di-quinoline substituted piperazine-piperidine compound of Formula V, such as those described in Scheme 1.

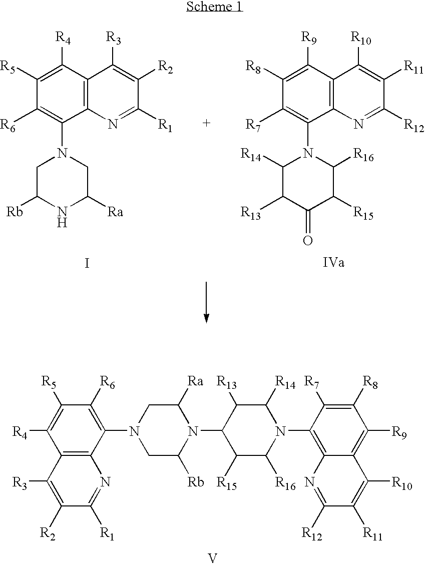

wherein R₁, R₂, R₃, R₄, R₅, R₆, R₇, R₈, R₉, R₁₀, R₁₁, R₁₂, R₁₃, R₁₄, R₁₅, and R₁₆, are each independently —H, (C₁-C₆)-alkyl, (C₁-C₆)-haloalkyl, (C₂-C₆)-alkenyl, or (C₂-C₆)-alkynyl, halogen, —CF₃, —NO₂, —CN, —OR₂₅, —OSO₂R₂₅, —SR₂₅, —SO₂R₂₅, —SO₂N(R₂₅)₂, —N(R₂₅)₂, C(O), —COR₂₅, —CO₂R₂₅, —NR₂₅CO₂R₂₅, —NR₂₅COR₂₅, —NR₂₅CON(R₂₅)₂, or —CON(R₂₅)₂;

Rₐ and R_{b} are each independently —H or —CH₃; and

R₂₅ is —H; or linear or branched (C₁-C₆)-alkyl, (C₁-C₆)-haloalkyl, (C₂-C₆)-alkenyl, or (C₂-C₆)-alkynyl.

Scheme 2 illustrates the production of compounds of Formula (I) and Formula (IV) in which R₁, R₂, R₃, R₁₀, R₁₁, R₁₂, R₁₃, R₁₄, R₁₅, and R₁₆ are each hydrogen and Rₐ, R_{b}, R₄, R₅, R₆, R₇, R₈ and R₉ are as defined above. An optionally substituted aniline compound of Formula IIc is reacted with an appropriate reagent under conditions effective to produce the quinoline compound of Formula IIIc. Numerous reagents and conditions affect this transformation. Many of these can be found in a review by G. Jones (Synthesis of the Quinoline Ring System, in Heterocyclic Compounds: Volume 32 (Quinoilines), Interscience, New York, N.Y., 1977, pp. 93-318). One such reagent is glycerol, as originally described by Skraup (Monatsh. (1880), 1, 316). R₄, R₅ and R₆ of IIc are as above for I and W is a good leaving group, for example halogen, p-toluenesulfonyl (—OTs), methanesulfonyl (—OMs) or trifluoromethanesulfonyl —OTr. The compound of Formula IIIc is then reacted with a protected-piperazine derivative under conditions effective to provide a protected piperazino-quinoline of Formula X, wherein A is a protecting group. Protecting groups are well known to those of skill in the art and include, without limitation, tert-butoxycarbonyl. Conditions that can effect this reaction include, but are not limited to, reacting the two components in the presence of a palladium complex such as those described by Buchwald et al., J. Am. Chem. Soc. 118:7215 (1996) and Hartwig et al., J. Am. Chem. Soc. 118:7217 (1996). The protected piperazino-quinoline of Formula X is then reacted under conditions to promote the removal of the protecting group (e.g., aqueous acid or mixtures of a water miscible organic solvent and aqueous acid), providing the substituted piperazino-quinoline compound of Formula I. Separately, compounds of Formula IV are produced by beginning with an optionally substituted aniline compound of Formula II and reacting it with glycerol under conditions effective to produce the quinoline compound of Formula III as described above. R₇, R₈ and R₉ are as above for Formula IVa and W is a suitable leaving group such as halogen, —OTs, —OMs or —OTr. The quinoline compound of Formula III is then reacted with a piperidin-4-one derivative where the carbonyl group is protected under conditions effective to provide the compound of Formula IX (e.g., a palladium-catalyzed coupling such as that described above). Suitable protecting groups are well known to those of skill in the art and include, without limitation, 1,4-dioxo-8-azaspiro-4,5-decane. The compound of Formula IX is then reacted under conditions to promote the removal of the protecting group (e.g., aqueous acid or a mixture of a water miscible organic solvent and aqueous acid), providing the piperidin-4-one compound of Formula IV. The piperidin-4-one compound of Formula IV is then reacted with the piperazino-quinoline compound of Formula I as described above in Schemes 1 and 2 to produce the di-quinoline piperazine-piperidine compound of Formula Vc.

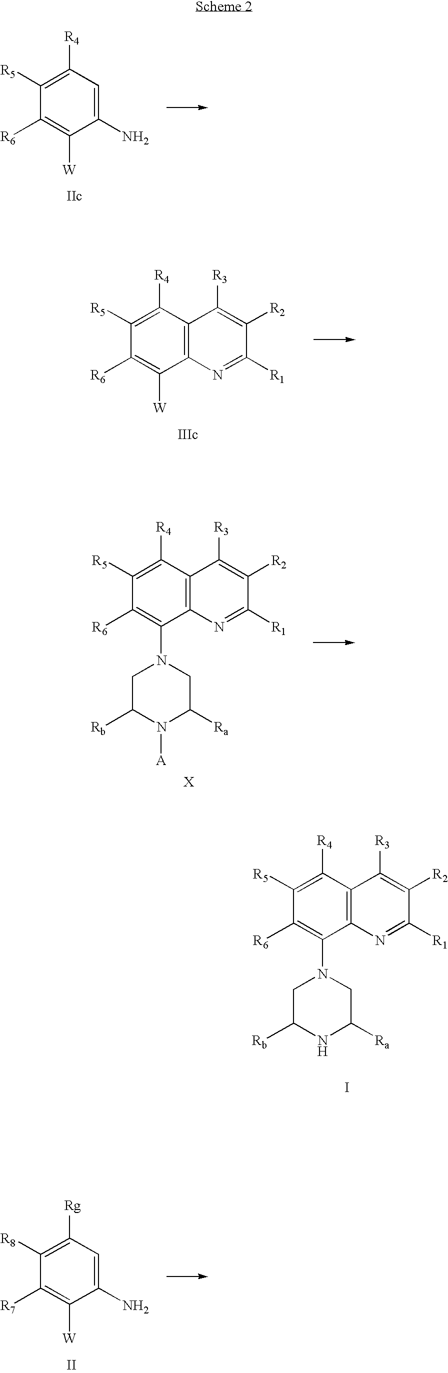

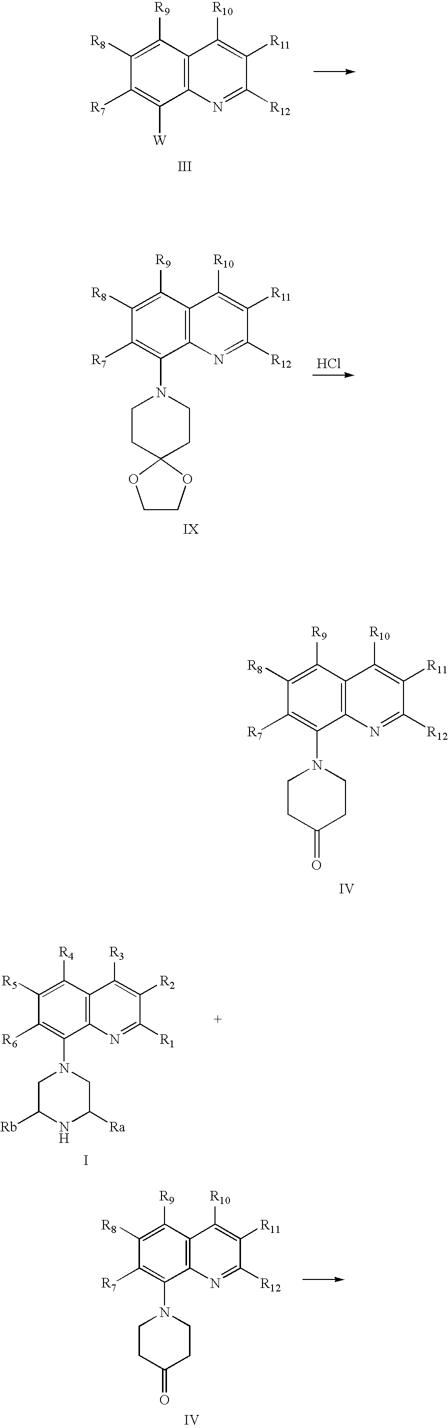

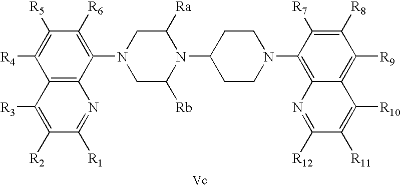

wherein R₁, R₂, R₃, R₄, R₅, R₆, R₇, R₈, R₉, R₁₀, R₁₁, and R₁₂, are each independently —H, (C₁-C₆)-alkyl, (C₁-C₆)-haloalkyl, (C₂-C₆)-alkenyl, or (C₂-C₆)-alkynyl, halogen, —CF₃, —NO₂, —CN, —OR₂₅, —OSO₂R₂₅, —SR₂₅, —SO₂R₂₅, —SO₂N(R₂₅)₂, —N(R₂₅)₂, C(O), —COR₂₅, —CO₂R₂₅, —NR₂₅CO₂R₂₅, —NR₂₅COR₂₅, —NR₂₅CON(R₂₅)₂, or —CON(R₂₅)₂;

Rₐ and R_{b} are each independently —H or —CH₃; and

R₂₅ is —H; or linear or branched (C₁-C₆)-alkyl, (C₁-C₆)-haloalkyl, (C₂-C₆)-alkenyl, or (C₂-C₆)-alkynyl.

Alternative syntheses for the quinoline compounds of Formulae IX and X are provided in Scheme 3, shown below. The aniline compounds of Formulae XII and XIII are reacted with an appropriate reagent under conditions effective to produce the quinoline compounds of Formulae IX and X. Reagents and conditions suitable for affecting this transformation are known to those of skill in the art and include, for example, methods described in G. Jones, supra. One exemplary reagent is glycerol. The compounds of Formulae XIV and XV are then reacted with appropriate reagents to yield the desired intermediate compounds of Formulae IX and X.

The methods of the present invention also provide means for more safely synthesizing piperazine-piperidine compounds that have toxic and environmentally damaging byproducts. The present invention provides the method of Scheme 2a in which the components of certain steps in the process have been altered to allow for safer synthesis of the compounds of interest. In certain embodiments, the reaction of optionally substituted anilines in conditions effective to form optionally substituted quinolines is performed by the process of Scheme 2a:

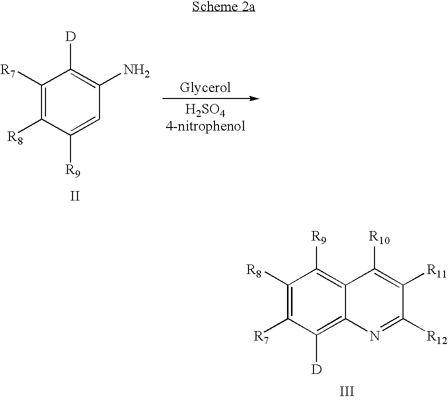

wherein D is a good leaving group, and R₇, R₈, R₉, R₁₀, R₁₁, and R₁₂, are each independently —H, (C₁-C₆)-alkyl, (C₁-C₆)-haloalkyl, (C₂-C₆)-alkenyl, or (C₂-C₆)-alkynyl, halogen, —CF₃, —NO₂, —CN, —OR₂₅, —OSO₂R₂₅, —SR₂₅, —SO₂R₂₅, —SO₂N(R₂₅)₂, —N(R₂₅)₂, C(O), —COR₂₅, —CO₂R₂₅, —NR₂₅CO₂R₂₅, —NR₂₅COR₂₅, —NR₂₅CON(R₂₅)₂, or —CON(R₂₅)₂;

Rₐ and R_{b} are each independently —H or —CH₃; and

R₂₅ is —H; or linear or branched (C₁-C₆)-alkyl, (C₁-C₆)-haloalkyl, (C₂-C₆)-alkenyl, or (C₂-C₆)-alkynyl.

In one embodiment, D is halogen.

According to the synthesis of Scheme 2a, 4-nitrophenol and optionally substituted aniline compounds of Formulae II are mixed together prior to addition to or the addition of acid to produce the quinoline compounds of Formula III. In certain embodiments, the acid is a strong acid. In other embodiments, the acid is H₂SO₄ or HCl. In one embodiment, glycerol is added to the reaction. By performing the present reaction accordingly, the reaction is less likely to represent a hazardous increase in temperature (e.g., thermal runaway). This reduces the possibility of a volatile reaction occurring, thereby improving the safety of the production process.

In additional embodiments, Scheme 2 is further modified to allow for isolation of piperazine-piperadine compounds without the use of potentially environmentally hazardous materials. In particular, piperadine and piperazine compounds are reacted in conditions in which toluene replaces chlorinated solvents to produce the following reaction Scheme 2b:

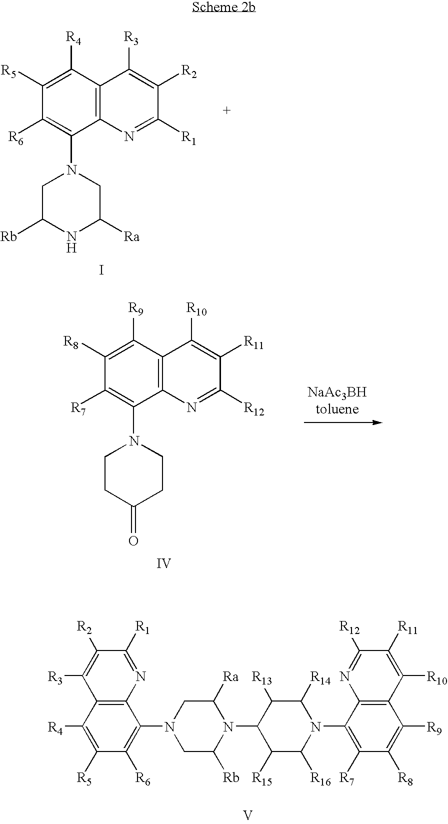

wherein R₁, R₂, R₃, R₄, R₅, R₆, R₇, R₈, R₉, R₁₀, R₁₁, R₁₂, R₁₃, R₁₄, R₁₅, and R₁₆ are each independently —H, (C₁-C₆)-alkyl, (C₁-C₆)-haloalkyl, (C₂-C₆)-alkenyl, or (C₂-C₆)-alkynyl, halogen, —CF₃, —NO₂, —CN, —OR₂₅, —OSO₂R₂₅, —SR₂₅, —SO₂R₂₅, —SO₂N(R₂₅)₂, —N(R₂₅)₂, C(O), —COR₂₅, —CO₂R₂₅, —NR₂₅CO₂R₂₅, —NR₂₅COR₂₅, —NR₂₅CON(R₂₅)₂, or —CON(R₂₅)₂;

Rₐ and R_{b} are each independently —H or —CH₃; and

R₂₅ is —H; or linear or branched (C₁-C₆)-alkyl, (C₁-C₆)-haloalkyl, (C₂-C₆)-alkenyl, or (C₂-C₆)-alkynyl.

The production of the di-quinoline substituted piperazine-piperidine compound of Formula V is accomplished by the reaction of the compounds of Formula I and Formula IV in effective conditions for the reaction to be completed. In certain embodiments, the use of toluene reduces the amount of chlorinated byproducts requiring disposal, which reduces the amount of hazardous environmental byproducts produced during synthesis of the piperazine-piperidine compounds. In addition, the use of toluene rather than chlorinated compounds such as CH₂Cl₂ reduces the toxicity of the compounds. Such decrease in toxicity is important due to the use of these compounds as pharmaceutical agents.

The process shown in Scheme 2b also has the advantage of increasing the yield of Formula V over processes utilizing CH₂Cl₂. In certain embodiments, the yield of the di-quinoline substituted piperazine-piperidine compound of Formula V in processes using toluene is between 2.5 times and 3 times greater than processes utilizing dichloromethane. In certain other embodiments, the yield is increased by 1.5 times to 2 times over processes utilizing dichloromethane. In other embodiments, the yield is increased by more than 3 times, and up to 10 times over processes using dichloromethane.

The isolation of quinoline-substituted piperazine compounds of Formula I has presented a problem during standard production procedures due to the generation of highly viscous solutions that create difficulties in isolating intermediates for further processing. Accordingly, the present invention provides methods of isolating the compounds of Formula I. In some embodiments, the reaction of Scheme 3 is modified to allow for improved isolation of di-quinoline substituted piperazine-piperidine compound of Formula V by way of Scheme 2c:

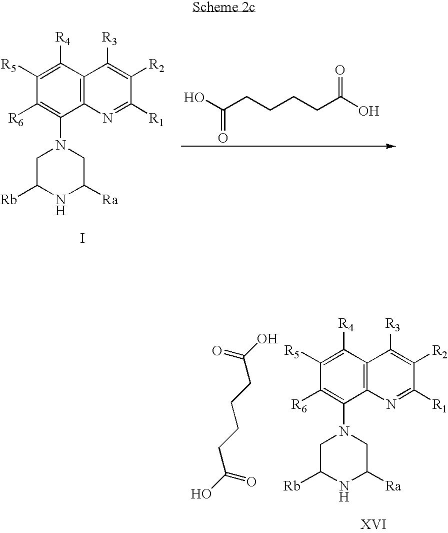

wherein R₁, R₂, R₃, R₄, R₅, and R₆, are each independently —H, (C₁-C₆)-alkyl, (C₁-C₆)-haloalkyl, (C₂-C₆)-alkenyl, or (C₂-C₆)-alkynyl, halogen, —CF₃, —NO₂, —CN, —OR₂₅, —OSO₂R₂₅, —SR₂₅, —SO₂R₂₅, —SO₂N(R₂₅)₂, —N(R₂₅)₂, C(O), —COR₂₅, —CO₂R₂₅, —NR₂₅CO₂R₂₅, —NR₂₅COR₂₅, —NR₂₅CON(R₂₅)₂, or —CON(R₂₅)₂;

Rₐ and R_{b} are each independently —H or —CH₃; and

R₂₅ is —H; or linear or branched (C₁-C₆)-alkyl, (C₁-C₆)-haloalkyl, (C₂-C₆)-alkenyl, or (C₂-C₆)-alkynyl.

The quinoline-substituted piperazine of Formula I is reacted with adipic acid in conditions effective to yield the adipate salt of the quinoline-substituted piperazine of Formula XVI. The reaction allows for further isolation of the quinoline-substituted piperazine in the presence of NaOH, toluene, CH₂Cl₂, and EtOAc according to Scheme 2d:

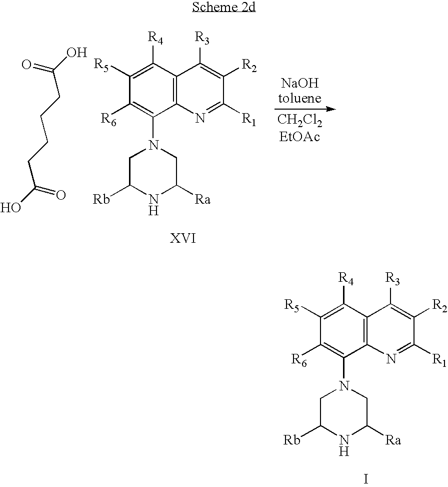

Accordingly, the present invention provides a method for isolating quinoline-substituted piperazine compounds of Formula XVI and Formula I.

In additional embodiments, Scheme 2 is modified to allow for isolation of piperazine-piperadine compounds without the use of potentially environmentally hazardous materials. In particular, piperadine and piperazine compounds are reacted in conditions in which toluene replaces chlorinated solvents to produce the following reaction Scheme 2e:

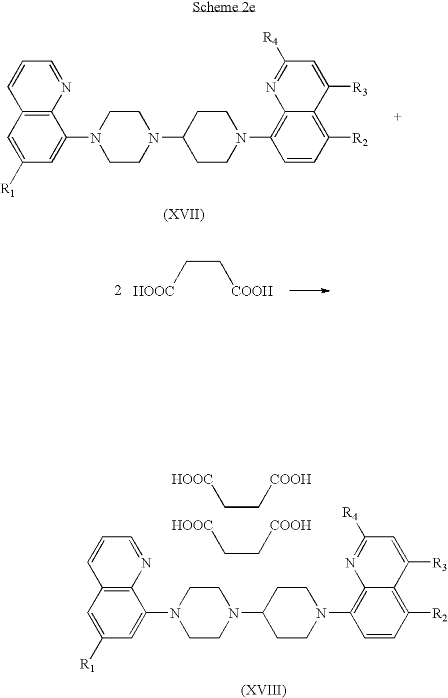

wherein R₁, R₂, R₃, and R₄, are each independently hydrogen, (C₁-C₆)-alkyl, (C₁-C₆)-haloalkyl, (C₂-C₆)-alkenyl, or (C₂-C₆)-alkynyl, halogen, —CF₃, —NO₂, —CN, —OR₂₅, —OSO₂R₂₅, —SR₂₅, —SO₂R₂₅, —SO₂N(R₂₅)₂, —N(R₂₅)₂, C(O), —COR₂₅, —CO₂R₂₅, —NR₂₅CO₂R₂₅, —NR₂₅COR₂₅, —NR₂₅CON(R₂₅)₂, or —CON(R₂₅)₂; and

R₂₅ is —H; or linear or branched (C₁-C₆)-alkyl, (C₁-C₆)-haloalkyl, (C₂-C₆)-alkenyl, or (C₂-C₆)-alkynyl.

The production of the di-quinoline substituted piperazine-piperidine compound of Formula (XXI) is accomplished as described above for compounds of Formula (I), (I′), and (I″).

In some embodiments, R₁, R₂, R₃, and R₄, are each independently hydrogen, (C₁-C₆)-alkyl, (C₁-C₆)-haloalkyl, (C₂-C₆)-alkenyl, or (C₂-C₆)-alkynyl, halogen, —CF₃, —OR₂₅, and R₂₅ is —H; or linear or branched (C₁-C₆)-alkyl, (C₁-C₆)-haloalkyl, (C₂-C₆)-alkenyl, or (C₂-C₆)-alkynyl. In certain embodiments, R₁, and R₃ are hydrogen, (C₁-C₆)-alkyl and R₂ is hydrogen or halogen (e.g., fluorine). In one embodiment, R₄, is hydrogen or —CF₃.

In certain embodiments, the process shown in Scheme 2e has the advantage of decreasing the amount of solvent found in the final compound. In some embodiments, the amount of each individual solvent is less than 0.25 w % of the compound identified in solution. In other embodiments, the amount of each solvent is less than 0.2 w % of the compound identified in solution. In still other embodiments, the amount of each solvent is less than 0.15 w % of the compound identified in solution. In further embodiments, the amount of each solvent is less than 0.1 w % of the compound identified in solution. In yet more embodiments, the amount of each solvent is less than 0.05 w % of the compound identified in solution. In still more embodiments, the amount of each solvent is less than 0.025 w % of the compound identified in solution. In additional embodiments, the amount of each solvent is less than 0.02 w % of the compound identified in solution. In another embodiment, the amount of each solvent is less than 0.01 w % of the compound identified in solution. In one embodiment, the presence of chlorinated solvents is decreased significantly from the final isolated compound.

In some embodiments, the process shown in Scheme 2e occurs in the presence of organic compounds including, but not limited to, THF, acetone, dichloromethane, and dichloroethane. In certain embodiments, the organic compounds are THF and acetone. In one embodiment, the compounds of Formula XVII are mixed with THF prior to addition to a solution of acetone and an organic acid. In another embodiment, the organic acid is succinic acid.

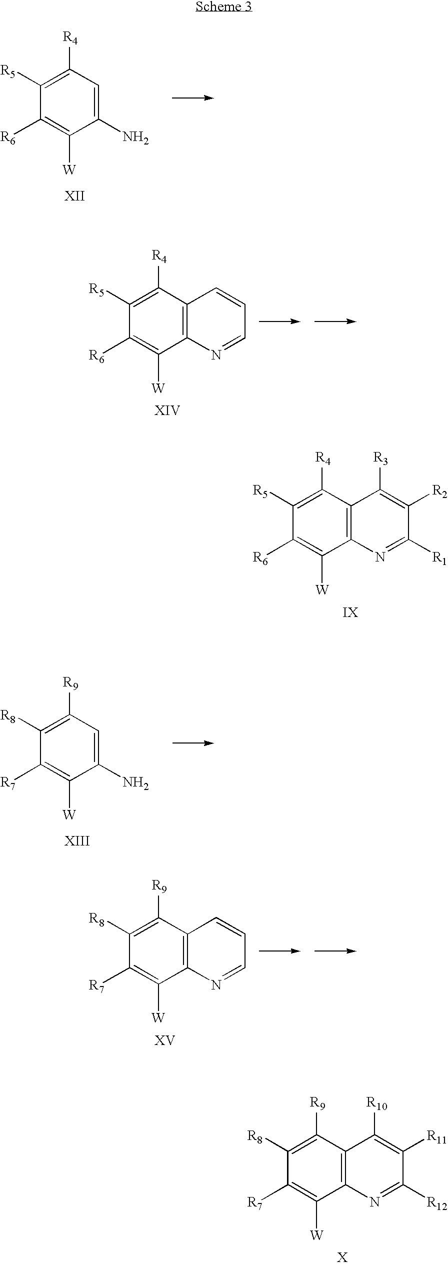

wherein W is halogen, and R₁, R₂, R₃, R₄, R₅, R₆, R₇, R₈, R₉, R₁₀, R₁₁, and R₁₂, are each independently —H, (C₁-C₆)-alkyl, (C₁-C₆)-haloalkyl, (C₂-C₆)-alkenyl, or (C₂-C₆)-alkynyl, halogen, —CF₃, —NO₂, —CN, —OR₂₅, —OSO₂R₂₅, —SR₂₅, —SO₂R₂₅, —SO₂N(R₂₅)₂, —N(R₂₅)₂, C(O), —COR₂₅, —CO₂R₂₅, —NR₂₅CO₂R₂₅, —NR₂₅COR₂₅, —NR₂₅CON(R₂₅)₂, or —CON(R₂₅)₂;

Rₐ and R_{b} are each independently —H or —CH₃; and

R₂₅ is —H; or linear or branched (C₁-C₆)-alkyl, (C₁-C₆)-haloalkyl, (C₂-C₆)-alkenyl, or (C₂-C₆)-alkynyl.

Schemes 1-4 illustrate the synthetic methodology used to prepare particular compounds of the present invention. One of skill in the art will recognize that Schemes 1-4 can be adapted to produce the other compounds according to the present invention and that other methods may be used to produce the compounds of the present invention.

Compounds of the Invention

The processes of synthesis described above are used to produce novel piperazine-piperidine compounds. In one embodiment, the process of the present invention is directed to synthesizing compounds of the Formula (V):

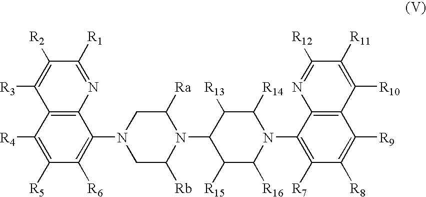

and pharmaceutically acceptable salts and hydrates thereof,

wherein R₁, R₂, R₃, R₄, R₅, R₆, R₇, R₈, R₉, R₁₀, R₁₁, R₁₂, R₁₃, R₁₄, R₁₅, and R₁₆, are each independently —H, (C₁-C₆)-alkyl, (C₁-C₆)-haloalkyl, (C₂-C₆)-alkenyl, or (C₂-C₆)-alkynyl, halogen, —CF₃, —NO₂, —CN, —OR₂₅, —OSO₂R₂₅, —SR₂₅, —SO₂R₂₅, —SO₂N(R₂₅)₂, —N(R₂₅)₂, C(O), —COR₂₅, —CO₂R₂₅, —NR₂₅CO₂R₂₅, —NR₂₅COR₂₅, —NR₂₅CON(R₂₅)₂, or —CON(R₂₅)₂;

Rₐ and R_{b} are each independently —H or —CH₃; and

R₂₅ is —H; or linear or branched (C₁-C₆)-alkyl, (C₁-C₆)-haloalkyl, (C₂-C₆)-alkenyl, or (C₂-C₆)-alkynyl.

In one embodiment, R₁ is (C₁-C₆)-alkyl, —OR₂₅, halogen, or —CF₃. In another embodiment, R₁ is (C₁-C₆)-alkyl, —OR₂₅, halogen, or —CF₃ and one of R₁₃, R₁₄, R₁₅, and R₁₆ is (C₁-C₆)-alkyl, —OR₂₅, or halogen. In a further embodiment, R₁ is (C₁-C₆)-alkyl, —OR₂₅, halogen, or —CF₃; one of R₁₃, R₁₄, R₁₅, and R₁₆ is (C₁-C₆)-alkyl, —OR₂₅, or halogen, and R₇, R₈, R₉, R₁₀, R₁₁, and R₁₂ are each hydrogen. In yet another embodiment, R₁ is (C₁-C₆)-alkyl, —OR₂₅, halogen, or —CF₃; one of R₁₃, R₁₄, R₁₅, and R₁₆ is (C₁-C₆)-alkyl, —OR₂₅, or halogen, and R₁, R₂, R₃, R₅, R₆, R₇, R₈, R₉, R₁₀, R₁₁, and R₁₂ are each hydrogen. In one embodiment, R₁ is (C₁-C₆)-alkyl, —OR₂₅, halogen or —CF₃ and R₁, R₂, R₃, R₅, R₆, R₇, R₈, R₉, R₁₀, R₁₁, R₁₂, R₁₃, R₁₄, R₁₅, and R₁₆ are each hydrogen.

In one embodiment, R₅ is (C₁-C₆)-alkyl, —OR₂₅, halogen, or —CF₃. In another embodiment, R₅ is (C₁-C₆)-alkyl, —OR₂₅, halogen, or —CF₃ and one of R₁₃, R₁₄, R₁₅, and R₁₆ is (C₁-C₆)-alkyl, —OR₂₅, or halogen. In a further embodiment, R₅ is (C₁-C₆)-alkyl, —OR₂₅, halogen, or —CF₃; one of R₁₃, R₁₄, R₁₅, and R₁₆ is (C₁-C₆)-alkyl, —OR₂₅, halogen or —CF₃, and R₇, R₈, R₉, R₁₀, R₁₁, and R₁₂ are each hydrogen. In yet another embodiment, R₅ is (C₁-C₆)-alkyl, —OR₂₅, halogen, or —CF₃; one of R₁₃, R₁₄, R₁₅, and R₁₆ is (C₁-C₆)-alkyl, —OR₂₅, or halogen; and R₁, R₂, R₃, R₄, R₆, R₇, R₈, R₉, R₁₀, R₁₁, and R₁₂ are each hydrogen. In one embodiment, R₅ is (C₁-C₆)-alkyl, —OR₂₅, halogen or —CF₃ and R₁, R₂, R₃, R₄, R₆, R₇, R₈, R₉, R₁₀, R₁₁, R₁₂, R₁₃, R₁₄, R₁₅, and R₁₆ are each hydrogen. In a further embodiment, one of R₁₃, R₁₄, R₁₅, and R₁₆ is (C₁-C₆)-alkyl, halogen, —CF₃, or —OR₂₅; R₅ is (C₁-C₆)-alkyl, —OR₂₅, halogen, or —CF₃; and the remaining substituents are each hydrogen.

In one embodiment, R₄ is (C₁-C₆)-alkyl, —OR₂₅, halogen, or —CF₃. In another embodiment, R₄ is (C₁-C₆)-alkyl, —OR₂₅, halogen, or —CF₃ and one of R₁₃, R₁₄, R₁₅, and R₁₆ is (C₁-C₆)-alkyl, —OR₂₅, or halogen. In a further embodiment, R₄ is (C₁-C₆)-alkyl, —OR₂₅, halogen, or —CF₃; one of R₁₃, R₁₄, R₁₅, and R₁₆ is (C₁-C₆)-alkyl, —OR₂₅, or halogen, and R₇, R₈, R₉, R₁₀, R₁₁, and R₁₂ are each hydrogen. In yet another embodiment, R₄ is (C₁-C₆)-alkyl, —OR₂₅, halogen, or —CF₃; one of R₁₃, R₁₄, R₁₅, and R₁₆ is (C₁-C₆)-alkyl, —OR₂₅, or halogen, and R₁, R₂, R₃, R₅, R₆, R₇, R₈, R₉, R₁₀, R₁₁, and R₁₂ are each hydrogen. In one embodiment, R₄ is (C₁-C₆)-alkyl, —OR₂₅, halogen or —CF₃ and R₁, R₂, R₃, R₅, R₆, R₇, R₈, R₉, R₁₀, R₁₁, R₁₂, R₁₃, R₁₄, R₁₅, and R₁₆ are each hydrogen.

In one embodiment, R₉ is (C₁-C₆)-alkyl, —OR₂₅, halogen, —CF₃, —NO₂ or —CN. In another embodiment, R₉ is (C₁-C₆)-alkyl, —OR₂₅, halogen, —CF₃, —NO₂ or —CN; one of R₁, R₂, R₃, R₄, R₅ and R₆ is (C₁-C₆)-alkyl, —OR₂₅, halogen, or —CF₃; and Rₐ and R_{b} are each independently —H or —CH₃; and the remaining substituents are each hydrogen. In a further embodiment, R₉ is (C₁-C₆)-alkyl, —OR₂₅, halogen, —CF₃, —NO₂ or —CN; one of R₁, R₂, R₃, R₄, R₅ and R₆ is (C₁-C₆)-alkyl, —OR₂₅, halogen, or —CF₃; one of R₁₃, R₁₄, R₁₅, and R₁₆ is (C₁-C₆)-alkyl, —OR₂₅, or halogen, and the remaining substituents are each hydrogen. In one embodiment, R₉ is (C₁-C₆)-alkyl, —OR₂₅, halogen, —CF₃, —NO₂ or —CN and one of R₄ or R₅ is (C₁-C₆)-alkyl, —OR₂₅, halogen, or —CF₃, and the remaining substituents are each hydrogen. In one embodiment R₉ is (C₁-C₆)-alkyl, —OR₂₅, halogen, —CF₃, —NO₂ or —CN; and all other R groups are each hydrogen. In one embodiment, R₉ is (C₁-C₆)-alkyl, —OR₂₅, halogen, —CF₃, —NO₂ or —CN; one of R₁₃, R₁₄, R₁₅, and R₁₆ is (C₁-C₆)-alkyl, —OR₂₅, or halogen, and the remaining substituents are each hydrogen.

In one embodiment, R₈ is (C₁-C₆)-alkyl, —OR₂₅, halogen, —CF₃, —NO₂ or —CN. In another embodiment, R₈ is (C₁-C₆)-alkyl, —OR₂₅, halogen, —CF₃, —NO₂ or —CN; one of R₁, R₂, R₃, R₄, R₅ and R₆ is (C₁-C₆)-alkyl, —OR₂₅, halogen, or —CF₃; Rₐ and R_{b} are each independently —H or —CH₃; and the remaining substituents are each hydrogen. In a further embodiment, R₈ is (C₁-C₆)-alkyl, —OR₂₅, halogen, —CF₃, —NO₂ or —CN; one of R₁, R₂, R₃, R₄, R₅ and R₆ is (C₁-C₆)-alkyl, —OR₂₅, halogen, or —CF₃; one of R₁₃, R₁₄, R₁₅, and R₁₆ is (C₁-C₆)-alkyl, —OR₂₅, or halogen, and the remaining substituents are each hydrogen. In one embodiment, R₈ is (C₁-C₆)-alkyl, —OR₂₅, halogen, —CF₃, —NO₂ or —CN and one of R₄ or R₅ is (C₁-C₆)-alkyl, —OR₂₅, halogen, or —CF₃, and the remaining substituents are each hydrogen. In one embodiment R₈ is (C₁-C₆)-alkyl, —OR₂₅, halogen, —CF₃, —NO₂ or —CN; and all other R groups are each hydrogen. In one embodiment, R₈ is (C₁-C₆)-alkyl, —OR₂₅, halogen, —CF₃, —NO₂ or —CN; one of R₁₃, R₁₄, R₁₅, and R₁₆ is (C₁-C₆)-alkyl, —OR₂₅, or halogen, and the remaining substituents are each hydrogen.

In one embodiment, R₇ is (C₁-C₆)-alkyl, —OR₂₅, halogen, —CF₃, —NO₂ or —CN. In one embodiment, R₇ is —OR₂₅ and R₂₅ is (C₁-C₆)-alkyl. In one embodiment, R₇ is —OCH₃

In one embodiment, R₁₀ is (C₁-C₆)-alkyl, —OR₂₅, halogen, —CF₃, —NO₂ or —CN.

In one embodiment, R₁₀ is —OR₂₅ and R₂₅ is (C₁-C₆)-alkyl. In one embodiment, R₁₀ is —OCH₃.

In one embodiment, R₁₁ is (C₁-C₆)-alkyl, —OR₂₅, halogen, —CF₃, —NO₂ or —CN. In one embodiment, R₁₁ is —OR₂₅ and R₂₅ is (C₁-C₆)-alkyl. In one embodiment, R₁₁ is —OCH₃

In one embodiment, R₁₂ is (C₁-C₆)-alkyl, —OR₂₅, halogen, —CF₃, —NO₂ or —CN. In one embodiment, R₁₂ is —CF₃.

In one embodiment, R₅ is (C₁-C₆)-alkyl, —OR₂₅, halogen, or —CF₃ and one of R₇, R₈, R₉, R₁₀, R₁₁ and R₁₂ is (C₁-C₆)-alkyl, —OR₂₅, halogen, —CF₃, —NO₂ or —CN. In another embodiment, R₅ is (C₁-C₆)-alkyl, —OR₂₅, halogen, or —CF₃ and one of R₇, R₈, R₉, R₁₀, R₁₁, R₁₂ is (C₁-C₆)-alkyl, —OR₂₅, halogen, —CF₃, —NO₂ or —CN; and the remaining substituents are each hydrogen. In some embodiments, R₅ is (C₁-C₆)-alkyl, —OR₂₅, halogen, or —CF₃ and R₉ is (C₁-C₆)-alkyl, —OR₂₅, halogen, —CF₃, —NO₂ or —CN; and the remaining substituents are each hydrogen.

In a further embodiment, R₅ is (C₁-C₆)-alkyl, —OR₂₅, halogen, or —CF₃; one of R₇, R₈, R₉, R₁₀, R₁₁, and R₁₂ is (C₁-C₆)-alkyl, —OR₂₅, halogen, —CF₃, —NO₂ or —CN; one of R₁₃, R₁₄, R₁₅, and R₁₆ is (C₁-C₆)-alkyl, —OR₂₅, or halogen, and the remaining substituents are each hydrogen.

In a further embodiment, R₅ is (C₁-C₆)-alkyl, —OR₂₅, halogen, or —CF₃; two of R₇, R₈, R₉, R₁₀, R₁₁, and R₁₂ is (C₁-C₆)-alkyl, —OR₂₅, halogen, —CF₃, —NO₂ or —CN; one of R₁₃, R₁₄, R₁₅, and R₁₆ is (C₁-C₆)-alkyl, —OR₂₅, or halogen, and the remaining substituents are each hydrogen.

In a further embodiment, R₅ is (C₁-C₆)-alkyl, —OR₂₅, halogen, or —CF₃; three of R₇, R₈, R₉, R₁₀, R₁₁, and R₁₂ is (C₁-C₆)-alkyl, —OR₂₅, halogen, —CF₃, —NO₂ or —CN; one of R₁₃, R₁₄, R₁₅, and R₁₆ is (C₁-C₆)-alkyl, —OR₂₅, or halogen, and the remaining substituents are each hydrogen.

In one embodiment, R₅ is (C₁-C₆)-alkyl, —OR₂₅, halogen, or —CF₃; R₉ is (C₁-C₆)-alkyl, —OR₂₅, halogen, —CF₃, —NO₂ or —CN; and two of R₁₀, R₁₁ and R₁₂ are each independently (C₁-C₆)-alkyl, —OR₂₅, halogen, —CF₃, —NO₂ or —CN. In another embodiment, R₅ is (C₁-C₆)-alkyl, —OR₂₅, halogen, or —CF₃; R₉ is (C₁-C₆)-alkyl, —OR₂₅, halogen, —CF₃, —NO₂ or —CN; two of R₁₀, R₁₁, R₁₂ is (C₁-C₆)-alkyl, —OR₂₅, halogen, —CF₃, —NO₂ or —CN; and the remaining substituents are each hydrogen. In some embodiments, R₅ is —OR₂₅; R₉ is halogen; two of R₁₀, R₁₁, R₁₂ is (C₁-C₆)-alkyl, —OR₂₅, halogen, —CF₃, —NO₂ or —CN; and the remaining substituents are each hydrogen. In some embodiments, R₅ is —OCH₃; R₉ is halogen; two of R₁₀, R₁₁, R₁₂ is (C₁-C₆)-alkyl, —OR₂₅, halogen, —CF₃, —NO₂ or —CN; and the remaining substituents are each hydrogen. In some embodiments, R₅ is (C₁-C₆)-alkyl, —OR₂₅, halogen, or —CF₃; R₉ is (C₁-C₆)-alkyl, —OR₂₅, halogen, —CF₃, —NO₂ or —CN; R₁₀ and R₁₂ are each independently (C₁-C₆)-alkyl, —OR₂₅, halogen, —CF₃, —NO₂ or —CN; and the remaining substituents are each hydrogen. In some embodiments, R₅ is (C₁-C₆)-alkyl, —OR₂₅, halogen, or —CF₃; R₉ is (C₁-C₆)-alkyl, —OR₂₅, halogen, —CF₃, —NO₂ or —CN; R₁₀ and R₁₁ are each independently (C₁-C₆)-alkyl, —OR₂₅, halogen, —CF₃, —NO₂ or —CN; and the remaining substituents are each hydrogen. In some embodiments, R₅ is (C₁-C₆)-alkyl, —OR₂₅, halogen, or —CF₃; R₉ is (C₁-C₆)-alkyl, —OR₂₅, halogen, —CF₃, NO₂ or —CN; R₁₁ and R₁₂ are each independently (C₁-C₆)-alkyl, —OR₂₅, halogen, —CF₃, —NO₂ or —CN; and the remaining substituents are each hydrogen.

In one embodiment, R₄ is (C₁-C₆)-alkyl, —OR₂₅, halogen, or —CF₃ and one of R₇, R₈, R₉, R₁₀, R₁₁ and R₁₂ is (C₁-C₆)-alkyl, —OR₂₅, halogen, —CF₃, —NO₂ or —CN. In another embodiment, R₄ is (C₁-C₆)-alkyl, —OR₂₅, halogen, or —CF₃; one of R₇, R₈, R₉, R₁₀, R₁₁, and R₁₂ is (C₁-C₆)-alkyl, —OR₂₅, halogen, —CF₃, —NO₂ or —CN; and the remaining substituents are each hydrogen. In a further embodiment, R₄ is (C₁-C₆)-alkyl, —OR₂₅, halogen, or —CF₃; one of R₇, R₈, R₉, R₁₀, R₁₁; R₁₂ is (C₁-C₆)-alkyl, —OR₂₅, halogen, —CF₃, —NO₂ or —CN; one of R₁₃, R₁₄, R₁₅, and R₁₆ is (C₁-C₆)-alkyl, —OR₂₅, or halogen, and the remaining substituents are each hydrogen.

In one embodiment, one of R₁₃, R₁₄, R₁₅, and R₁₆ is (C₁-C₆)-alkyl, halogen, —CF₃, or —OR₂₅. In one embodiment, R₉ is (C₁-C₆)-alkyl, —OR₂₅, halogen, —CF₃, —NO₂ or —CN. In another embodiment, R₉ is (C₁-C₆)-alkyl, —OR₂₅, halogen, —CF₃, —NO₂ or —CN; one of R₁, R₂, R₃, R₄, R₅ and R₆ is (C₁-C₆)-alkyl, —OR₂₅, halogen, or —CF₃; and Rₐ and R_{b} are each independently —H or —CH₃; and the remaining substituents are each hydrogen. In a further embodiment, R₉ is (C₁-C₆)-alkyl, —OR₂₅, halogen, —CF₃, —NO₂ or —CN; one of R₁, R₂, R₃, R₄, R₅ and R₆ is (C₁-C₆)-alkyl, —OR₂₅, halogen, or —CF₃; one of R₁₃, R₁₄, R₁₅, and R₁₆ is (C₁-C₆)-alkyl, —OR₂₅, or halogen, and the remaining substituents are each hydrogen. In one embodiment, R₉ is (C₁-C₆)-alkyl, —OR₂₅, halogen, —CF₃, —NO₂ or —CN and one of R₄ or R₅ is (C₁-C₆)-alkyl, —OR₂₅, halogen, or —CF₃, and the remaining substituents are each hydrogen. In one embodiment R₉ is (C₁-C₆)-alkyl, —OR₂₅, halogen, —CF₃, —NO₂ or —CN; and all other R groups are each hydrogen. In one embodiment, R₉ is (C₁-C₆)-alkyl, —OR₂₅, halogen, —CF₃, —NO₂ or —CN; one of R₁₃, R₁₄, R₁₅, and R₁₆ is (C₁-C₆)-alkyl, —OR₂₅, or halogen, and the remaining substituents are each hydrogen.

In one embodiment, R₈ is (C₁-C₆)-alkyl, —OR₂₅, halogen, —CF₃, —NO₂ or —CN. In another embodiment, R₈ is (C₁-C₆)-alkyl, —OR₂₅, halogen, —CF₃, —NO₂ or —CN; one of R₁, R₂, R₃, R₄, R₅ and R₆ is (C₁-C₆)-alkyl, —OR₂₅, halogen, or —CF₃; Rₐ and R_{b} are each independently —H or —CH₃; and the remaining substituents are each hydrogen. In a further embodiment, R₈ is (C₁-C₆)-alkyl, —OR₂₅, halogen, —CF₃, —NO₂ or —CN; one of R₁, R₂, R₃, R₄, R₅ and R₆ is (C₁-C₆)-alkyl, —OR₂₅, halogen, or —CF₃; one of R₁₃, R₁₄, R₁₅, and R₁₆ is (C₁-C₆)-alkyl, —OR₂₅, or halogen, and the remaining substituents are each hydrogen. In one embodiment, R₈ is (C₁-C₆)-alkyl, —OR₂₅, halogen, —CF₃, —NO₂ or —CN and one of R₄ or R₅ is (C₁-C₆)-alkyl, —OR₂₅, halogen, or —CF₃, and the remaining substituents are each hydrogen. In one embodiment R₈ is (C₁-C₆)-alkyl, —OR₂₅, halogen, —CF₃, —NO₂ or —CN; and all other R groups are each hydrogen. In one embodiment, R₈ is (C₁-C₆)-alkyl, —OR₂₅, halogen, —CF₃, —NO₂ or —CN; one of R₁₃, R₁₄, R₁₅, and R₁₆ is (C₁-C₆)-alkyl, —OR₂₅, or halogen, and the remaining substituents are each hydrogen.

In one embodiment, R₇ is (C₁-C₆)-alkyl, —OR₂₅, halogen, —CF₃, —NO₂ or —CN. In one embodiment, R₇ is —OR₂₅ and R₂₅ is (C₁-C₆)-alkyl. In one embodiment, R₇ is —OCH₃.

In one embodiment, R₁₀ is (C₁-C₆)-alkyl, —OR₂₅, halogen, —CF₃, —NO₂ or —CN. In one embodiment, R₁₀ is —OR₂₅ and R₂₅ is (C₁-C₆)-alkyl. In one embodiment, R₁₀ is —OCH₃.

In one embodiment, R₁₁ is (C₁-C₆)-alkyl, —OR₂₅, halogen, —CF₃, —NO₂ or —CN. In one embodiment, R₁₁ is —OR₂₅ and R₂₅ is (C₁-C₆)-alkyl. In one embodiment, R₁₁ is —OCH₃

In one embodiment, R₁₂ is (C₁-C₆)-alkyl, —OR₂₅, halogen, —CF₃, —NO₂ or —CN. In one embodiment, R₁₂ is —CF₃.

In one embodiment, R₁, R₂, R₃, R₆, R₇, R₈, R₉, R₁₀, R₁₁, R₁₂, R₁₃, R₁₄, R₁₅, and R₁₆ are each hydrogen.

In one embodiment, R₁, R₂, R₃, R₄, R₇, R₉, R₁₀, R₁₁, and R₁₂ are each hydrogen.

In one embodiment, R₁, R₂, R₃, R₄, R₇, R₈, R₁₀, R₁₁, and R₁₂ are each hydrogen.

In one embodiment, R₁, R₂, R₃, R₄, R₇, R₈, R₉, R₁₁, and R₁₂ are each hydrogen.

In one embodiment, R₁, R₂, R₃, R₄, R₇, R₈, R₉, R₁₀, and R₁₂ are each hydrogen.

In one embodiment, R₁, R₂, R₃, R₄, R₇, R₈, R₉, R₁₀, and R₁₁ are each hydrogen.

In one embodiment, R₁, R₂, R₃, R₄, R₇, R₈, and R₁₁ are each hydrogen.

In one embodiment, R₁, R₂, R₃, R₄, R₇, R₈, R₉ and R₁₁ are each hydrogen.

In one embodiment, R₁, R₂, R₃, R₄, R₅, R₆, R₇, R₈, R₉, and R₁₂ are each hydrogen.

In another embodiment, R₁₃, R₁₄, R₁₅, and R₁₆ are each hydrogen.

In one embodiment, R₃, R₆, R₇, R₈, R₉, R₁₂, R₁₃, R₁₄, R₁₅, and R₁₆ are each hydrogen.

In one embodiment, R₁ is —H or (C₁-C₆)-alkyl; R₂, R₈, and R₉ are each —H or halogen; R₄ is —H, halogen, —OR₂₅, or —CF₃; R₅ is —H, halogen, or —OR₂₅; and R₃, R₆, R₇, R₁₂, R₁₃, R₁₄, R₁₅, R₁₆, Rₐ and R_{b} are each hydrogen. In one embodiment, R₁ is —H or —CH₃; R₂, R₈, and R₉ are each —H or F; R₄ is —H, F, —OCH₃, or —CF₃; R₅ is —H, F, —OCH₃; and R₃, R₆, R₇, R₁₀, R₁₁, R₁₂, R₁₃, R₁₄, R₁₅, R₁₆, Rₐ and R_{b} are each hydrogen.

In one embodiment, R₁ is —H, —CF₃ or (C₁-C₆)-alkyl; R₄ and R₅ are each —H, halogen, —OR₂₅, or —CF₃; R₇, R₈, R₉, R₁₀, R₁₁, and R₁₂ are each —H, halogen, -alkyl, —OR₂₅, —CF₃, or —NO₂; R₁₆ is —H or —CH₃.

In one embodiment, any one of R₇, R₈, R₉, R₁₀, R₁₁, and R₁₂ is (C₁-C₆)-alkyl, —OR₂₅, halogen, —CF₃, —NO₂, or —CN; and any one of R₁₃, R₁₄, R₁₅, and R₁₆ is (C₁-C₆)-alkyl, —OR₂₅, halogen, —CF₃.

In one embodiment, any one of R₁, R₂, R₃, R₄, R₅, and R₆ is (C₁-C₆)-alkyl, —OR₂₅, halogen or —CF₃; and any one of R₇, R₈, R₉, R₁₀, R₁₁, and R₁₂ is (C₁-C₆)-alkyl, —OR₂₅, halogen, or —CF₃, —NO₂, or —CN.

In one embodiment, any one of R₁, R₂, R₃, R₄, R₅, and R₆ is (C₁-C₆)-alkyl, —OR₂₅, halogen or —CF₃; and any one of R₁₃, R₁₄, R₁₅, and R₁₆ is (C₁-C₆)-alkyl, —OR₂₅, halogen or —CF₃.

In one embodiment, R₄ is (C₁-C₆)-alkyl, —OR₂₅, halogen, or —CF₃ and any one of R₁₃, R₁₄, R₁₅, and R₁₆ is (C₁-C₆)-alkyl, —OR₂₅, halogen or —CF₃; and any one of R₇, R₈, R₉, R₁₀, R₁₁, and R₁₂ is (C₁-C₆)-alkyl, —OR₂₅, halogen, or —CF₃, —NO₂, or —CN.

In one embodiment, R₄ is (C₁-C₆)-alkyl, —OR₂₅, halogen, or —CF₃ and any one of R₁₃, R₁₄, R₁₅, and R₁₆ is (C₁-C₆)-alkyl, —OR₂₅, halogen or —CF₃; and any two of R₇, R₈, R₉, R₁₀, R₁₁, and R₁₂ is (C₁-C₆)-alkyl, —OR₂₅, halogen, or —CF₃, —NO₂, or —CN; wherein the any two of R₇, R₈, R₉, R₁₀, R₁₁, and R₁₂ can be either on the same ring of the quinoline or on different rings.

In one embodiment, R₅ is (C₁-C₆)-alkyl, —OR₂₅, halogen, or —CF₃ and any one of R₁₃, R₁₄, R₁₅, and R₁₆ is (C₁-C₆)-alkyl, —OR₂₅, halogen or —CF₃; and any one of R₇, R₈, R₉, R₁₀, R₁₁, and R₁₂ is (C₁-C₆)-alkyl, —OR₂₅, halogen, or —CF₃, —NO₂, or —CN.

In one embodiment, R₅ is (C₁-C₆)-alkyl, —OR₂₅, halogen, or —CF₃ and any one of R₁₃, R₁₄, R₁₅, and R₁₆ is (C₁-C₆)-alkyl, —OR₂₅, halogen or —CF₃; and any two of R₇, R₈, R₉, R₁₀, R₁₁, and R₁₂ is (C₁-C₆)-alkyl, —OR₂₅, halogen, or —CF₃, —NO₂, or —CN; wherein the any two of R₇, R₈, R₉, R₁₀, R₁₁, and R₁₂ can be either on the same ring of the quinoline or on different rings.

In one embodiment, R₂₅ is (C₁-C₆)-haloalkyl.

In another embodiment, R₂₅ is (C₁-C₆)-fluoroalkyl.

In one embodiment, R₂₅ is (C₁-C₆)-alkyl. In one embodiment, R₂₅ is —CH₃.

In one embodiment, the compounds of Formula (V) are antagonists of the 5-HT_{1A} receptor. In another embodiment, the compounds of Formula (V) are agonists of the 5-HT_{1A} receptor.

In another aspect, the processes for synthesizing piperazine-piperidine compounds provides compounds of the Formula (Vc):

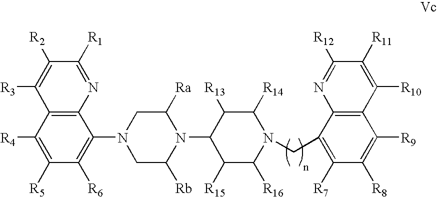

and pharmaceutically acceptable salts and hydrates thereof,

wherein R₁, R₂, R₃, R₄, R₅, R₆, R₇, R₈, R₉, R₁₀, R₁₁, R₁₂, R₁₃, R₁₄, R₁₅, and R₁₆, are each independently —H, (C₁-C₆)-alkyl, (C₁-C₆)-haloalkyl, (C₂-C₆)-alkenyl, or (C₂-C₆)-alkynyl, halogen, —CF₃, —NO₂, —CN, —OR₂₅, —OSO₂R₂₅, —SR₂₅, —SO₂R₂₅, —SO₂N(R₂₅)₂, —N(R₂₅)₂, C(O), —COR₂₅, —CO₂R₂₅, —NR₂₅CO₂R₂₅, —NR₂₅COR₂₅, —NR₂₅CON(R₂₅)₂, or —CON(R₂₅)₂;

Rₐ and R_{b} are each independently —H or —CH₃;

R₂₅ is —H; or linear or branched (C₁-C₆)-alkyl, (C₁-C₆)-haloalkyl, (C₂-C₆)-alkenyl, or (C₂-C₆)-alkynyl; and n is an integer from 1 to 2.

In one embodiment, R₁ is (C₁-C₆)-alkyl, —OR₂₅, halogen, or —CF₃. In another embodiment, R₁ is (C₁-C₆)-alkyl, —OR₂₅, halogen, or —CF₃ and one of R₁₃, R₁₄, R₁₅, and R₁₆ is (C₁-C₆)-alkyl, —OR₂₅, or halogen. In a further embodiment, R₁ is (C₁-C₆)-alkyl, —OR₂₅, halogen, or —CF₃; one of R₁₃, R₁₄, R₁₅, and R₁₆ is (C₁-C₆)-alkyl, —OR₂₅, or halogen, and R₇, R₈, R₉, R₁₀, R₁₁, and R₁₂ are each hydrogen. In yet another embodiment, R₁ is (C₁-C₆)-alkyl, —OR₂₅, halogen, or —CF₃; one of R₁₃, R₁₄, R₁₅, and R₁₆ is (C₁-C₆)-alkyl, —OR₂₅, or halogen, and R₁, R₂, R₃, R₅, R₆, R₇, R₈, R₉, R₁₀, R₁₁, and R₁₂ are each hydrogen. In one embodiment, R₁ is (C₁-C₆)-alkyl, —OR₂₅, halogen or —CF₃ and R₁, R₂, R₃, R₅, R₆, R₇, R₈, R₉, R₁₀, R₁₁, R₁₂, R₁₃, R₁₄, R₁₅, and R₁₆ are each hydrogen.

In one embodiment, R₅ is (C₁-C₆)-alkyl, —OR₂₅, halogen, or —CF₃. In another embodiment, R₅ is (C₁-C₆)-alkyl, —OR₂₅, halogen, or —CF₃ and one of R₁₃, R₁₄, R₁₅, and R₁₆ is (C₁-C₆)-alkyl, —OR₂₅, or halogen. In a further embodiment, R₅ is (C₁-C₆)-alkyl, —OR₂₅, halogen, or —CF₃; one of R₁₃, R₁₄, R₁₅, and R₁₆ is (C₁-C₆)-alkyl, —OR₂₅, halogen or —CF₃, and R₇, R₈, R₉, R₁₀, R₁₁, and R₁₂ are each hydrogen. In yet another embodiment, R₅ is (C₁-C₆)-alkyl, —OR₂₅, halogen, or —CF₃; one of R₁₃, R₁₄, R₁₅, and R₁₆ is (C₁-C₆)-alkyl, —OR₂₅, or halogen; and R₁, R₂, R₃, R₄, R₆, R₇, R₈, R₉, R₁₀, R₁₁, and R₁₂ are each hydrogen. In one embodiment, R₅ is (C₁-C₆)-alkyl, —OR₂₅, halogen or —CF₃ and R₁, R₂, R₃, R₄, R₆, R₇, R₈, R₉, R₁₀, R₁₁, R₁₂, R₁₃, R₁₄, R₁₅, and R₁₆ are each hydrogen. In a further embodiment, one of R₁₃, R₁₄, R₁₅, and R₁₆ is (C₁-C₆)-alkyl, halogen, —CF₃, or —OR₂₅; R₅ is (C₁-C₆)-alkyl, —OR₂₅, halogen, or —CF₃; and the remaining substituents are each hydrogen.

In one embodiment, R₄ is (C₁-C₆)-alkyl, —OR₂₅, halogen, or —CF₃. In another embodiment, R₄ is (C₁-C₆)-alkyl, —OR₂₅, halogen, or —CF₃ and one of R₁₃, R₁₄, R₁₅, and R₁₆ is (C₁-C₆)-alkyl, —OR₂₅, or halogen. In a further embodiment, R₄ is (C₁-C₆)-alkyl, —OR₂₅, halogen, or —CF₃; one of R₁₃, R₁₄, R₁₅, and R₁₆ is (C₁-C₆)-alkyl, —OR₂₅, or halogen, and R₇, R₈, R₉, R₁₀, R₁₁, and R₁₂ are each hydrogen. In yet another embodiment, R₄ is (C₁-C₆)-alkyl, —OR₂₅, halogen, or —CF₃; one of R₁₃, R₁₄, R₁₅, and R₁₆ is (C₁-C₆)-alkyl, —OR₂₅, or halogen, and R₁, R₂, R₃, R₅, R₆, R₇, R₈, R₉, R₁₀, R₁₁, and R₁₂ are each hydrogen. In one embodiment, R₄ is (C₁-C₆)-alkyl, —OR₂₅, halogen or —CF₃ and R₁, R₂, R₃, R₅, R₆, R₇, R₈, R₉, R₁₀, R₁₁, R₁₂, R₁₃, R₁₄, R₁₅, and R₁₆ are each hydrogen.

In one embodiment, R₉ is (C₁-C₆)-alkyl, —OR₂₅, halogen, —CF₃, —NO₂ or —CN. In another embodiment, R₉ is (C₁-C₆)-alkyl, —OR₂₅, halogen, —CF₃, —NO₂ or —CN; one of R₁, R₂, R₃, R₄, R₅ and R₆ is (C₁-C₆)-alkyl, —OR₂₅, halogen, or —CF₃; and Rₐ and R_{b} are each independently —H or —CH₃; and the remaining substituents are each hydrogen. In a further embodiment, R₉ is (C₁-C₆)-alkyl, —OR₂₅, halogen, —CF₃, —NO₂ or —CN; one of R₁, R₂, R₃, R₄, R₅ and R₆ is (C₁-C₆)-alkyl, —OR₂₅, halogen, or —CF₃; one of R₁₃, R₁₄, R₁₅, and R₁₆ is (C₁-C₆)-alkyl, —OR₂₅, or halogen, and the remaining substituents are each hydrogen. In one embodiment, R₉ is (C₁-C₆)-alkyl, —OR₂₅, halogen, —CF₃, —NO₂ or —CN and one of R₄ or R₅ is (C₁-C₆)-alkyl, —OR₂₅, halogen, or —CF₃, and the remaining substituents are each hydrogen. In one embodiment R₉ is (C₁-C₆)-alkyl, —OR₂₅, halogen, —CF₃, —NO₂ or —CN; and all other R groups are each hydrogen. In one embodiment, R₉ is (C₁-C₆)-alkyl, —OR₂₅, halogen, —CF₃, —NO₂ or —CN; one of R₁₃, R₁₄, R₁₅, and R₁₆ is (C₁-C₆)-alkyl, —OR₂₅, or halogen, and the remaining substituents are each hydrogen.

In one embodiment, R₈ is (C₁-C₆)-alkyl, —OR₂₅, halogen, —CF₃, —NO₂ or —CN. In another embodiment, R₈ is (C₁-C₆)-alkyl, —OR₂₅, halogen, —CF₃, —NO₂ or —CN; one of R₁, R₂, R₃, R₄, R₅ and R₆ is (C₁-C₆)-alkyl, —OR₂₅, halogen, or —CF₃; Rₐ and R_{b} are each independently —H or —CH₃; and the remaining substituents are each hydrogen. In a further embodiment, R₈ is (C₁-C₆)-alkyl, —OR₂₅, halogen, —CF₃, —NO₂ or —CN; one of R₁, R₂, R₃, R₄, R₅ and R₆ is (C₁-C₆)-alkyl, —OR₂₅, halogen, or —CF₃; one of R₁₃, R₁₄, R₁₅, and R₁₆ is (C₁-C₆)-alkyl, —OR₂₅, or halogen, and the remaining substituents are each hydrogen. In one embodiment, R₈ is (C₁-C₆)-alkyl, —OR₂₅, halogen, —CF₃, —NO₂ or —CN and one of R₄ or R₅ is (C₁-C₆)-alkyl, —OR₂₅, halogen, or —CF₃, and the remaining substituents are each hydrogen. In one embodiment R₈ is (C₁-C₆)-alkyl, —OR₂₅, halogen, —CF₃, —NO₂ or —CN; and all other R groups are each hydrogen. In one embodiment, R₈ is (C₁-C₆)-alkyl, —OR₂₅, halogen, —CF₃, —NO₂ or —CN; one of R₁₃, R₁₄, R₁₅, and R₁₆ is (C₁-C₆)-alkyl, —OR₂₅, or halogen, and the remaining substituents are each hydrogen.

In one embodiment, R₇ is (C₁-C₆)-alkyl, —OR₂₅, halogen, —CF₃, —NO₂ or —CN. In one embodiment, R₇ is —OR₂₅ and R₂₅ is (C₁-C₆)-alkyl. In one embodiment, R₇ is —OCH₃

In one embodiment, R₁₀ is (C₁-C₆)-alkyl, —OR₂₅, halogen, —CF₃, —NO₂ or —CN. In one embodiment, R₁₀ is —OR₂₅ and R₂₅ is (C₁-C₆)-alkyl. In one embodiment, R₁₀ is —OCH₃.

In one embodiment, R₁ is (C₁-C₆)-alkyl, —OR₂₅, halogen, —CF₃, —NO₂ or —CN. In one embodiment, R₁₁ is —OR₂₅ and R₂₅ is (C₁-C₆)-alkyl. In one embodiment, R₁₁ is —OCH₃

In one embodiment, R₁₂ is (C₁-C₆)-alkyl, —OR₂₅, halogen, —CF₃, —NO₂ or —CN. In one embodiment, R₁₂ is —CF₃.

In one embodiment, R₅ is (C₁-C₆)-alkyl, —OR₂₅, halogen, or —CF₃ and one of R₇, R₈, R₉, R₁₀, R₁₁, and R₁₂ is (C₁-C₆)-alkyl, —OR₂₅, halogen, —CF₃, —NO₂ or —CN. In another embodiment, R₅ is (C₁-C₆)-alkyl, —OR₂₅, halogen, or —CF₃ and one of R₇, R₈, R₉, R₁₀, R₁₁, R₁₂ is (C₁-C₆)-alkyl, —OR₂₅, halogen, —CF₃, —NO₂ or —CN; and the remaining substituents are each hydrogen. In some embodiments, R₅ is (C₁-C₆)-alkyl, —OR₂₅, halogen, or —CF₃ and R₉ is (C₁-C₆)-alkyl, —OR₂₅, halogen, —CF₃, —NO₂ or —CN; and the remaining substituents are each hydrogen.

In a further embodiment, R₅ is (C₁-C₆)-alkyl, —OR₂₅, halogen, or —CF₃; one of R₇, R₈, R₉, R₁₀, R₁₁, and R₁₂ is (C₁-C₆)-alkyl, —OR₂₅, halogen, —CF₃, —NO₂ or —CN; one of R₁₃, R₁₄, R₁₅, and R₁₆ is (C₁-C₆)-alkyl, —OR₂₅, or halogen, and the remaining substituents are each hydrogen.

In a further embodiment, R₅ is (C₁-C₆)-alkyl, —OR₂₅, halogen, or —CF₃; two of R₇, R₈, R₉, R₁₀, R₁₁, and R₁₂ is (C₁-C₆)-alkyl, —OR₂₅, halogen, —CF₃, —NO₂ or —CN; one of R₁₃, R₁₄, R₁₅, and R₁₆ is (C₁-C₆)-alkyl, —OR₂₅, or halogen, and the remaining substituents are each hydrogen.

In a further embodiment, R₅ is (C₁-C₆)-alkyl, —OR₂₅, halogen, or —CF₃; three of R₇, R₈, R₉, R₁₀, R₁₁, and R₁₂ is (C₁-C₆)-alkyl, —OR₂₅, halogen, —CF₃, —NO₂ or —CN; one of R₁₃, R₁₄, R₁₅, and R₁₆ is (C₁-C₆)-alkyl, —OR₂₅, or halogen, and the remaining substituents are each hydrogen.

In one embodiment, R₅ is (C₁-C₆)-alkyl, —OR₂₅, halogen, or —CF₃; R₉ is (C₁-C₆)-alkyl, —OR₂₅, halogen, —CF₃, —NO₂ or —CN; and two of R₁₀, R₁₁ and R₁₂ are each independently (C₁-C₆)-alkyl, —OR₂₅, halogen, —CF₃, —NO₂ or —CN. In another embodiment, R₅ is (C₁-C₆)-alkyl, —OR₂₅, halogen, or —CF₃; R₉ is (C₁-C₆)-alkyl, —OR₂₅, halogen, —CF₃, —NO₂ or —CN; two of R₁₀, R₁₁, R₁₂ is (C₁-C₆)-alkyl, —OR₂₅, halogen, —CF₃, —NO₂ or —CN; and the remaining substituents are each hydrogen. In some embodiments, R₅ is —OR₂₅; R₉ is halogen; two of R₁₀, R₁₁, R₁₂ is (C₁-C₆)-alkyl, —OR₂₅, halogen, —CF₃, —NO₂ or —CN; and the remaining substituents are each hydrogen. In some embodiments, R₅ is —OCH₃; R₉ is halogen; two of R₁₀, R₁₁, R₁₂ is (C₁-C₆)-alkyl, —OR₂₅, halogen, —CF₃, —NO₂ or —CN; and the remaining substituents are each hydrogen. In some embodiments, R₅ is (C₁-C₆)-alkyl, —OR₂₅, halogen, or —CF₃; R₉ is (C₁-C₆)-alkyl, —OR₂₅, halogen, —CF₃, —NO₂ or —CN; R₁₀ and R₁₂ are each independently (C₁-C₆)-alkyl, —OR₂₅, halogen, —CF₃, —NO₂ or —CN; and the remaining substituents are each hydrogen. In some embodiments, R₅ is (C₁-C₆)-alkyl, —OR₂₅, halogen, or —CF₃; R₉ is (C₁-C₆)-alkyl, —OR₂₅, halogen, —CF₃, —NO₂ or —CN; R₁₀ and R₁₁ are each independently (C₁-C₆)-alkyl, —OR₂₅, halogen, —CF₃, —NO₂ or —CN; and the remaining substituents are each hydrogen. In some embodiments, R₅ is (C₁-C₆)-alkyl, —OR₂₅, halogen, or —CF₃; R₉ is (C₁-C₆)-alkyl, —OR₂₅, halogen, —CF₃, —NO₂ or —CN; R₁₁ and R₁₂ are each independently (C₁-C₆)-alkyl, —OR₂₅, halogen, —CF₃, —NO₂ or —CN; and the remaining substituents are each hydrogen.

In one embodiment, R₄ is (C₁-C₆)-alkyl, —OR₂₅, halogen, or —CF₃ and one of R₇, R₈, R₉, R₁₀, R₁₁ and R₁₂ is (C₁-C₆)-alkyl, —OR₂₅, halogen, —CF₃, —NO₂ or —CN. In another embodiment, R₄ is (C₁-C₆)-alkyl, —OR₂₅, halogen, or —CF₃; one of R₇, R₈, R₉, R₁₀, R₁₁, and R₁₂ is (C₁-C₆)-alkyl, —OR₂₅, halogen, —CF₃, —NO₂ or —CN; and the remaining substituents are each hydrogen. In a further embodiment, R₄ is (C₁-C₆)-alkyl, —OR₂₅, halogen, or —CF₃; one of R₇, R₈, R₉, R₁₀, R₁₁; R₁₂ is (C₁-C₆)-alkyl, —OR₂₅, halogen, —CF₃, —NO₂ or —CN; one of R₁₃, R₁₄, R₁₅, and R₁₆ is (C₁-C₆)-alkyl, —OR₂₅, or halogen, and the remaining substituents are each hydrogen.

In one embodiment, one of R₁₃, R₁₄, R₁₅, and R₁₆ is (C₁-C₆)-alkyl, halogen, —CF₃, or —OR₂₅.

In one embodiment, R₉ is (C₁-C₆)-alkyl, —OR₂₅, halogen, —CF₃, —NO₂ or —CN. In another embodiment, R₉ is (C₁-C₆)-alkyl, —OR₂₅, halogen, —CF₃, —NO₂ or —CN; one of R₁, R₂, R₃, R₄, R₅ and R₆ is (C₁-C₆)-alkyl, —OR₂₅, halogen, or —CF₃; and Rₐ and R_{b} are each independently —H or —CH₃; and the remaining substituents are each hydrogen. In a further embodiment, R₉ is (C₁-C₆)-alkyl, —OR₂₅, halogen, —CF₃, —NO₂ or —CN; one of R₁, R₂, R₃, R₄, R₅ and R₆ is (C₁-C₆)-alkyl, —OR₂₅, halogen, or —CF₃; one of R₁₃, R₁₄, R₁₅, and R₁₆ is (C₁-C₆)-alkyl, —OR₂₅, or halogen, and the remaining substituents are each hydrogen. In one embodiment, R₉ is (C₁-C₆)-alkyl, —OR₂₅, halogen, —CF₃, —NO₂ or —CN and one of R₄ or R₅ is (C₁-C₆)-alkyl, —OR₂₅, halogen, or —CF₃, and the remaining substituents are each hydrogen. In one embodiment R₉ is (C₁-C₆)-alkyl, —OR₂₅, halogen, —CF₃, —NO₂ or —CN; and all other R groups are each hydrogen. In one embodiment, R₉ is (C₁-C₆)-alkyl, —OR₂₅, halogen, —CF₃, —NO₂ or —CN; one of R₁₃, R₁₄, R₁₅, and R₁₆ is (C₁-C₆)-alkyl, —OR₂₅, or halogen, and the remaining substituents are each hydrogen.

In one embodiment, R₈ is (C₁-C₆)-alkyl, —OR₂₅, halogen, —CF₃, —NO₂ or —CN. In another embodiment, R₈ is (C₁-C₆)-alkyl, —OR₂₅, halogen, —CF₃, —NO₂ or —CN; one of R₁, R₂, R₃, R₄, R₅ and R₆ is (C₁-C₆)-alkyl, —OR₂₅, halogen, or —CF₃; Rₐ and R_{b} are each independently —H or —CH₃; and the remaining substituents are each hydrogen. In a further embodiment, R₈ is (C₁-C₆)-alkyl, —OR₂₅, halogen, —CF₃, —NO₂ or —CN; one of R₁, R₂, R₃, R₄, R₅ and R₆ is (C₁-C₆)-alkyl, —OR₂₅, halogen, or —CF₃; one of R₁₃, R₁₄, R₁₅, and R₁₆ is (C₁-C₆)-alkyl, —OR₂₅, or halogen, and the remaining substituents are each hydrogen. In one embodiment, R₈ is (C₁-C₆)-alkyl, —OR₂₅, halogen, —CF₃, —NO₂ or —CN and one of R₄ or R₅ is (C₁-C₆)-alkyl, —OR₂₅, halogen, or —CF₃, and the remaining substituents are each hydrogen. In one embodiment R₈ is (C₁-C₆)-alkyl, —OR₂₅, halogen, —CF₃, —NO₂ or —CN; and all other R groups are each hydrogen. In one embodiment, R₈ is (C₁-C₆)-alkyl, —OR₂₅, halogen, —CF₃, —NO₂ or —CN; one of R₁₃, R₁₄, R₁₅, and R₁₆ is (C₁-C₆)-alkyl, —OR₂₅, or halogen, and the remaining substituents are each hydrogen.

In one embodiment, R₇ is (C₁-C₆)-alkyl, —OR₂₅, halogen, —CF₃, —NO₂ or —CN. In one embodiment, R₇ is —OR₂₅ and R₂₅ is (C₁-C₆)-alkyl. In one embodiment, R₇ is OCH₃

In one embodiment, R₁₀ is (C₁-C₆)-alkyl, —OR₂₅, halogen, —CF₃, —NO₂ or —CN. In one embodiment, R₁₀ is —OR₂₅ and R₂₅ is (C₁-C₆)-alkyl. In one embodiment, R₁₀ is—OCH₃.

In one embodiment, R₁₁ is (C₁-C₆)-alkyl, —OR₂₅, halogen, —CF₃, —NO₂ or —CN. In one embodiment, R₁₁ is —OR₂₅ and R₂₅ is (C₁-C₆)-alkyl. In one embodiment, R₁₁ is —OCH₃

In one embodiment, R₁₂ is (C₁-C₆)-alkyl, —OR₂₅, halogen, —CF₃, —NO₂ or —CN. In one embodiment, R₁₂ is —CF₃.

In one embodiment, R₁, R₂, R₃, R₆, R₇, R₈, R₉, R₁₀, R₁₁, R₁₂, R₁₃, R₁₄, R₁₅, and R₁₆ are each hydrogen.

In one embodiment, R₁, R₂, R₃, R₄, R₇, R₉, R₁₀, R₁₁, and R₁₂ are each hydrogen.

In one embodiment, R₁, R₂, R₃, R₄, R₇, R₈, R₁₀, R₁₁, and R₁₂ are each hydrogen.

In one embodiment, R₁, R₂, R₃, R₄, R₇, R₈, R₉, R₁₁, and R₁₂ are each hydrogen.

In one embodiment, R₁, R₂, R₃, R₄, R₇, R₈, R₉, R₁₀, and R₁₂ are each hydrogen.

In one embodiment, R₁, R₂, R₃, R₄, R₇, R₈, R₉, R₁₀, and R₁₁, are each hydrogen.

In one embodiment, R₁, R₂, R₃, R₄, R₇, R₈, and R₁₁, are each hydrogen.

In one embodiment, R₁, R₂, R₃, R₄, R₇, R₈, R₉ and R₁₁ are each hydrogen.

In one embodiment, R₁, R₂, R₃, R₄, R₅, R₆, R₇, R₈, R₉, and R₁₂ are each hydrogen.

In another embodiment, R₁₃, R₁₄, R₁₅, and R₁₆ are each hydrogen.

In one embodiment, R₃, R₆, R₇, R₈, R₉, R₁₂, R₁₃, R₁₄, R₁₅, and R₁₆ are each hydrogen.

In one embodiment, R₁ is —H or (C₁-C₆)-alkyl; R₂, R₈, and R₉ are each —H or halogen; R₄ is —H, halogen, —OR₂₅, or —CF₃; R₅ is —H, halogen, or —OR₂₅; and R₃, R₆, R₇, R₁₂, R₁₃, R₁₄, R₁₅, R₁₆, Rₐ and R_{b} are each hydrogen. In one embodiment, R₁ is —H or —CH₃; R₂, R₈, and R₉ are each —H or F; R₄ is —H, F, —OCH₃, or —CF₃; R₅ is —H, F, —OCH₃; and R₃, R₆, R₇, R₁₀, R₁₁, R₁₂, R₁₃, R₁₄, R₁₅, R₁₆, Rₐ and R_{b} are each hydrogen.

In one embodiment, R₁ is —H, —CF₃ or (C₁-C₆)-alkyl; R₄ and R₅ are each —H, halogen, —OR₂₅, or —CF₃; R₇, R₈, R₉, R₁₀, R₁₁, and R₁₂ are each —H, halogen, -alkyl, —OR₂₅, —CF₃, or —NO₂; R₁₆ is —H or —CH₃.

In one embodiment, any one of R₇, R₈, R₉, R₁₀, R₁₁, and R₁₂ is (C₁-C₆)-alkyl, —OR₂₅, halogen, —CF₃, —NO₂, or —CN; and any one of R₁₃, R₁₄, R₁₅, and R₁₆ is (C₁-C₆)-alkyl, —OR₂₅, halogen, —CF₃.

In one embodiment, any one of R₁, R₂, R₃, R₄, R₅, and R₆ is (C₁-C₆)-alkyl, —OR₂₅, halogen or —CF₃; and any one of R₇, R₈, R₉, R₁₀, R₁₁, and R₁₂ is (C₁-C₆)-alkyl, —OR₂₅, halogen, or —CF₃, —NO₂, or —CN.

In one embodiment, any one of R₁, R₂, R₃, R₄, R₅, and R₆ is (C₁-C₆)-alkyl, —OR₂₅, halogen or —CF₃; and any one of R₁₃, R₁₄, R₁₅, and R₁₆ is (C₁-C₆)-alkyl, —OR₂₅, halogen or —CF₃.

In one embodiment, R₄ is (C₁-C₆)-alkyl, —OR₂₅, halogen, or —CF₃ and any one of R₁₃, R₁₄, R₁₅, and R₁₆ is (C₁-C₆)-alkyl, —OR₂₅, halogen or —CF₃; and any one of R₇, R₈, R₉, R₁₀, R₁₁, and R₁₂ is (C₁-C₆)-alkyl, —OR₂₅, halogen, or —CF₃, —NO₂, or —CN.

In one embodiment, R₄ is (C₁-C₆)-alkyl, —OR₂₅, halogen, or —CF₃ and any one of R₁₃, R₁₄, R₁₅, and R₁₆ is (C₁-C₆)-alkyl, —OR₂₅, halogen or —CF₃; and any two of R₇, R₈, R₉, R₁₀, R₁₁, and R₁₂ is (C₁-C₆)-alkyl, —OR₂₅, halogen, or —CF₃, —NO₂, or —CN; wherein the any two of R₇, R₈, R₉, R₁₀, R₁₁, and R₁₂ can be either on the same ring of the quinoline or on different rings.

In one embodiment, R₅ is (C₁-C₆)-alkyl, —OR₂₅, halogen, or —CF₃ and any one of R₁₃, R₁₄, R₁₅, and R₁₆ is (C₁-C₆)-alkyl, —OR₂₅, halogen or —CF₃; and any one of R₇, R₈, R₉, R₁₀, R₁₁, and R₁₂ is (C₁-C₆)-alkyl, —OR₂₅, halogen, or —CF₃, —NO₂, or —CN.

In one embodiment, R₅ is (C₁-C₆)-alkyl, —OR₂₅, halogen, or —CF₃ and any one of R₁₃, R₁₄, R₁₅, and R₁₆ is (C₁-C₆)-alkyl, —OR₂₅, halogen or —CF₃; and any two of R₇, R₈, R₉, R₁₀, R₁₁, and R₁₂ is (C₁-C₆)-alkyl, —OR₂₅, halogen, or —CF₃, —NO₂, or —CN; wherein the any two of R₇, R₈, R₉, R₁₀, R₁₁, and R₁₂ can be either on the same ring of the quinoline or on different rings.

In one embodiment, R₂₅ is (C₁-C₆)-haloalkyl.

In another embodiment, R₂₅ is (C₁-C₆)-fluoroalkyl.

In one embodiment, R₂₅ is (C₁-C₆)-alkyl. In one embodiment, R₂₅ is —CH₃.

In one embodiment, the compounds of Formula V are antagonists of the 5-HT_{1A} receptor. In another embodiment, the compounds of Formula V are agonists of the 5-HT_{1A} receptor.

In another aspect, the compounds of the Formula Vd are synthesized by the methods of the present invention:

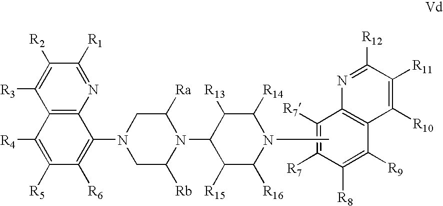

and pharmaceutically acceptable salts thereof,

wherein R₁, R₂, R₃, R₄, R₅, R₆, R₇, R_{7′}, R₈, R₉, R₁₀, R₁₁, R₁₂, R₁₃, R₁₄, R₁₅, and R₁₆, are each independently —H, (C₁-C₆)-alkyl, (C₁-C₆)-haloalkyl, (C₂-C₆)-alkenyl, or (C₂-C₆)-alkynyl, halogen, —CF₃, —NO₂, —CN, —OR₂₅, —OSO₂R₂₅, —SR₂₅, —SO₂R₂₅, —SO₂N(R₂₅)₂, —N(R₂₅)₂, C(O), —COR₂₅, —CO₂R₂₅, —NR₂₅CO₂R₂₅, —NR₂₅COR₂₅, —NR₂₅CON(R₂₅)₂, or —CON(R₂₅)₂;

Rₐ and R_{b} are each independently —H or —CH₃; and

R₂₅ is —H; or linear or branched (C₁-C₆)-alkyl, (C₁-C₆)-haloalkyl, (C₂-C₆)-alkenyl, or (C₂-C₆)-alkynyl; and

where the piperidine group can be attached to the non-hetero atom containing ring of the quinoline through positions R₇, R_{7′}, R₈, or R₉.

In one embodiment, R₅ is (C₁-C₆)-alkyl, —OR₂₅, halogen, or —CF₃. In another embodiment, R₅ is (C₁-C₆)-alkyl, —OR₂₅, halogen, or —CF₃ and one of R₁₃, R₁₄, R₁₅, and R₁₆ is (C₁-C₆)-alkyl, —OR₂₅, or halogen. In a further embodiment, R₅ is (C₁-C₆)-alkyl, —OR₂₅, halogen, or —CF₃; one of R₁₃, R₁₄, R₁₅, and R₁₆ is (C₁-C₆)-alkyl, —OR₂₅, halogen or —CF₃; the piperidine is connected through one of R₇, R_{7′}, R₈, or R₉; and the remainder of the R groups of the quinoline attached to the piperidine are each hydrogen. In yet another embodiment, R₅ is (C₁-C₆)-alkyl, —OR₂₅, halogen, or —CF₃; one of R₁₃, R₁₄, R₁₅, and R₁₆ is (C₁-C₆)-alkyl, —OR₂₅, or halogen; the piperidine is connected through R_{7′}; and the remaining R groups are each hydrogen. In yet another embodiment, R₅ is (C₁-C₆)-alkyl, —OR₂₅, halogen, or —CF₃; one of R₁₃, R₁₄, R₁₅, and R₁₆ is (C₁-C₆)-alkyl, —OR₂₅, or halogen; the piperidine is connected through R₇; and the remaining R groups are each hydrogen. In yet another embodiment, R₅ is (C₁-C₆)-alkyl, —OR₂₅, halogen, or —CF₃; one of R₁₃, R₁₄, R₁₅, and R₁₆ is (C₁-C₆)-alkyl, —OR₂₅, or halogen; the piperidine is connected through R₈; and the remaining R groups are each hydrogen. In yet another embodiment, R₅ is (C₁-C₆)-alkyl, —OR₂₅, halogen, or —CF₃; one of R₁₃, R₁₄, R₁₅, and R₁₆ is (C₁-C₆)-alkyl, —OR₂₅, or halogen; the piperidine is connected through R₉; and the remaining R groups are each hydrogen. In one embodiment, R₅ is (C₁-C₆)-alkyl, —OR₂₅, halogen or —CF₃, the piperidine is connected through R_{7′}, and the remaining R groups are each hydrogen. In one embodiment, R₅ is (C₁-C₆)-alkyl, —OR₂₅, halogen or —CF₃, the piperidine is connected through R₇, and the remaining R groups are each hydrogen. In one embodiment, R₅ is (C₁-C₆)-alkyl, —OR₂₅, halogen or —CF₃, the piperidine is connected through R₈, and the remaining R groups are each hydrogen. In one embodiment, R₅ is (C₁-C₆)-alkyl, —OR₂₅, halogen or —CF₃, the piperidine is connected through R₉, and the remaining R groups are each hydrogen. In a further embodiment, one of R₁₃, R₁₄, R₁₅, and R₁₆ is (C₁-C₆)-alkyl, halogen, —CF₃, or —OR₂₅; R₅ is (C₁-C₆)-alkyl, —OR₂₅, halogen, or —CF₃; and the remaining substituents are each hydrogen.

In one embodiment, R₄ is (C₁-C₆)-alkyl, —OR₂₅, halogen, or —CF₃. In another embodiment, R₄ is (C₁-C₆)-alkyl, —OR₂₅, halogen, or —CF₃ and one of R₁₃, R₁₄, R₁₅, and R₁₆ is (C₁-C₆)-alkyl, —OR₂₅, halogen, or —CF₃. In a further embodiment, R₄ is (C₁-C₆)-alkyl, —OR₂₅, halogen, or —CF₃; one of R₁₃, R₁₄, R₁₅, and R₁₆ is (C₁-C₆)-alkyl, —OR₂₅, halogen or —CF₃; the piperidine is connected through one of R₇, R_{7′}, R₈, or R₉; and the remainder of the R groups of the quinoline attached to the piperidine are each hydrogen. In yet another embodiment, R₄ is (C₁-C₆)-alkyl, —OR₂₅, halogen, or —CF₃; one of R₁₃, R₁₄, R₁₅, and R₁₆ is (C₁-C₆)-alkyl, —OR₂₅, or halogen; the piperidine is connected through R_{7′}; and the remaining R groups are each hydrogen. In yet another embodiment, R₄ is (C₁-C₆)-alkyl, —OR₂₅, halogen, or —CF₃; one of R₁₃, R₁₄, R₁₅, and R₁₆ is (C₁-C₆)-alkyl, —OR₂₅, or halogen; the piperidine is connected through R₇; and the remaining R groups are each hydrogen. In yet another embodiment, R₄ is (C₁-C₆)-alkyl, —OR₂₅, halogen, or —CF₃; one of R₁₃, R₁₄, R₁₅, and R₁₆ is (C₁-C₆)-alkyl, —OR₂₅, or halogen; the piperidine is connected through R₈; and the remaining R groups are each hydrogen. In yet another embodiment, R₅ is (C₁-C₆)-alkyl, —OR₂₅, halogen, or —CF₃; one of R₁₃, R₁₄, R₁₅, and R₁₆ is (C₁-C₆)-alkyl, —OR₂₅, or halogen; the piperidine is connected through R₉; and the remaining R groups are each hydrogen. In one embodiment, R₄ is (C₁-C₆)-alkyl, —OR₂₅, halogen or —CF₃, the piperidine is connected through R_{7′}, and the remaining R groups are each hydrogen. In one embodiment, R₄ is (C₁-C₆)-alkyl, —OR₂₅, halogen or —CF₃, the piperidine is connected through R₇, and the remaining R groups are each hydrogen. In one embodiment, R₄ is (C₁-C₆)-alkyl, —OR₂₅, halogen or —CF₃, the piperidine is connected through R₈, and the remaining R groups are each hydrogen. In one embodiment, R₄ is (C₁-C₆)-alkyl, —OR₂₅, halogen or —CF₃, the piperidine is connected through R₉, and the remaining R groups are each hydrogen.

In one embodiment, R₅ is (C₁-C₆)-alkyl, —OR₂₅, halogen, or —CF₃ and one of R₇, R₈, R₉, R₁₀, R₁₁ and R₁₂ is (C₁-C₆)-alkyl, —OR₂₅, halogen, —CF₃, —NO₂ or —CN. In another embodiment, R₅ is (C₁-C₆)-alkyl, —OR₂₅, halogen, or —CF₃ and one of R₇, R₈, R₉, R₁₀, R₁₁, or R₁₂ is (C₁-C₆)-alkyl, —OR₂₅, halogen, —CF₃, —NO₂ or —CN; and the remaining substituents are each hydrogen. In some embodiments, R₅ is —OR₂₅ and R₉ is (C₁-C₆)-alkyl, —OR₂₅, halogen, —CF₃, —NO₂ or —CN; and the remaining substituents are each hydrogen. In some embodiments, R₅ is (C₁-C₆)-alkyl, —OR₂₅, halogen, or —CF₃, R₉ is halogen and the remaining substituents are each hydrogen.

In a further embodiment, R₅ is (C₁-C₆)-alkyl, —OR₂₅, halogen, or —CF₃; one of R₇, R₈, R₉, R₁₀, R₁₁; R₁₂ is (C₁-C₆)-alkyl, —OR₂₅, halogen, —CF₃, —NO₂ or —CN; one of R₁₃, R₁₄, R₁₅, and R₁₆ is (C₁-C₆)-alkyl, —OR₂₅, or halogen, and the remaining substituents are each hydrogen.

In one embodiment, R₅ is (C₁-C₆)-alkyl, —OR₂₅, halogen, or —CF₃; two of R₇, R₈, R₉, R₁₀, R₁₁; R₁₂ are each independently (C₁-C₆)-alkyl, —OR₂₅, halogen, —CF₃, —NO₂ or —CN; one of R₁₃, R₁₄, R₁₅, and R₁₆ is (C₁-C₆)-alkyl, —OR₂₅, or halogen, and the remaining substituents are each hydrogen.

In one embodiment, R₅ is (C₁-C₆)-alkyl, —OR₂₅, halogen, or —CF₃; three of R₇, R₈, R₉, R₁₀, R₁₁; R₁₂ are each independently (C₁-C₆)-alkyl, —OR₂₅, halogen, —CF₃, —NO₂ or —CN; one of R₁₃, R₁₄, R₁₅, and R₁₆ is (C₁-C₆)-alkyl, —OR₂₅, or halogen, and the remaining substituents are each hydrogen.

In one embodiment, R₅ is (C₁-C₆)-alkyl, —OR₂₅, halogen, or —CF₃; R₉ is (C₁-C₆)-alkyl, —OR₂₅, halogen, —CF₃, —NO₂ or —CN; and two of R₁₀, R₁₁ and R₁₂ are each independently (C₁-C₆)-alkyl, —OR₂₅, halogen, —CF₃, —NO₂ or —CN. In another embodiment, R₅ is (C₁-C₆)-alkyl, —OR₂₅, halogen, or —CF₃; R₉ is (C₁-C₆)-alkyl, —OR₂₅, halogen, —CF₃, —NO₂ or —CN; two of R₁₀, R₁₁, R₁₂ is (C₁-C₆)-alkyl, —OR₂₅, halogen, —CF₃, —NO₂ or —CN; and the remaining substituents are each hydrogen. In some embodiments, In some embodiments, R₅ is —(C₁-C₆)-alkyl, —OR₂₅, halogen, or —CF₃; R₉ is (C₁-C₆)-alkyl, —OR₂₅, halogen, —CF₃, —NO₂ or —CN; R₁₀ and R₁₂ are each independently (C₁-C₆)-alkyl, —OR₂₅, halogen, —CF₃, —NO₂ or —CN; and the remaining substituents are each hydrogen. In some embodiments, In some embodiments, R₅ is —(C₁-C₆)-alkyl, —OR₂₅, halogen, or —CF₃; R₉ is (C₁-C₆)-alkyl, —OR₂₅, halogen, —CF₃, —NO₂ or —CN; R₁₀ and R₁₁ are each independently (C₁-C₆)-alkyl, —OR₂₅, halogen, —CF₃, —NO₂ or —CN; and the remaining substituents are each hydrogen. In some embodiments, In some embodiments, R₅ is —(C₁-C₆)-alkyl, —OR₂₅, halogen, or —CF₃; R₉ is (C₁-C₆)-alkyl, —OR₂₅, halogen, —CF₃, —NO₂ or —CN; R₁₁, and R₁₂ are each independently (C₁-C₆)-alkyl, —OR₂₅, halogen, —CF₃, —NO₂ or —CN; and the remaining substituents are each hydrogen.

In one embodiment, R₄ is (C₁-C₆)-alkyl, —OR₂₅, halogen, or —CF₃ and one of R_{7′}, R₇, R₈, R₉, R₁₀, R₁₁, and R₁₂ is (C₁-C₆)-alkyl, —OR₂₅, halogen, —CF₃, —NO₂ or —CN. In another embodiment, R₄ is (C₁-C₆)-alkyl, —OR₂₅, halogen, or —CF₃; one of R_{7′}, R₇, R₈, R₉, R₁₀, R₁₁, and R₁₂ is (C₁-C₆)-alkyl, —OR₂₅, halogen, —CF₃, —NO₂ or —CN; and the remaining substituents are each hydrogen. In a further embodiment, R₄ is (C₁-C₆)-alkyl, —OR₂₅, halogen, or —CF₃; one of R_{7′}, R₇, R₈, R₉, R₁₀, R₁₁; R₁₂ is (C₁-C₆)-alkyl, —OR₂₅, halogen, —CF₃, —NO₂ or —CN; one of R₁₃, R₁₄, R₁₅, and R₁₆ is (C₁-C₆)-alkyl, —OR₂₅, or halogen, and the remaining substituents are each hydrogen.

In one embodiment, one of R₁₃, R₁₄, R₁₅, and R₁₆ is (C₁-C₆)-alkyl, halogen, —CF₃, or —OR₂₅.

In one embodiment, R₉ is (C₁-C₆)-alkyl, —OR₂₅, halogen, —CF₃, —NO₂ or —CN. In another embodiment, R₉ is (C₁-C₆)-alkyl, —OR₂₅, halogen, —CF₃, —NO₂ or —CN; one of R₁, R₂, R₃, R₄, and R₆, is (C₁-C₆)-alkyl, —OR₂₅, halogen, or —CF₃; Rₐ and R_{b} are each independently —H or —CH₃; and the remaining substituents are each hydrogen except for the R group through which the piperidine is connected. In a further embodiment, R₉ is (C₁-C₆)-alkyl, —OR₂₅, halogen, —CF₃, —NO₂ or —CN; one of R₁, R₂, R₃, R₄, and R₆ is (C₁-C₆)-alkyl, —OR₂₅, halogen, or —CF₃; one of R₁₃, R₁₄, R₁₅, and R₁₆ is (C₁-C₆)-alkyl, —OR₂₅, or halogen; Rₐ and R_{b} are each independently —H or —CH₃; and the remaining substituents are each hydrogen except for the R group through which the piperidine is connected. In one embodiment, R₉ is (C₁-C₆)-alkyl, —OR₂₅, halogen, —CF₃, —NO₂ or —CN and one of R₄ or R₅ is (C₁-C₆)-alkyl, —OR₂₅, halogen, or —CF₃. In one embodiment R₉ is (C₁-C₆)-alkyl, —OR₂₅, halogen, —CF₃, —NO₂ or —CN; and all other R groups are each hydrogen except for the R group through which the piperidine is connected. In one embodiment, R₉ is (C₁-C₆)-alkyl, —OR₂₅, halogen, —CF₃, —NO₂ or —CN; one of R₁₃, R₁₄, R₁₅, and R₁₆ is (C₁-C₆)-alkyl, —OR₂₅, or halogen, and the remaining substituents are each hydrogen except for the R group through which the piperidine is connected.

In one embodiment, R₈ is (C₁-C₆)-alkyl, —OR₂₅, halogen, —CF₃, —NO₂ or —CN. In another embodiment, R₈ is (C₁-C₆)-alkyl, —OR₂₅, halogen, —CF₃, —NO₂ or —CN; one of R₁, R₂, R₃, R₄, and R₆ is (C₁-C₆)-alkyl, —OR₂₅, halogen, or —CF₃; Rₐ and R_{b} are each independently —H or —CH₃; and the remaining substituents are each hydrogen except for the R group through which the piperidine is connected. In a further embodiment, R₈ is (C₁-C₆)-alkyl, —OR₂₅, halogen, —CF₃, —NO₂ or —CN; one of R₁, R₂, R₃, R₄, and R₆ is (C₁-C₆)-alkyl, —OR₂₅, halogen, or —CF₃; one of R₁₃, R₁₄, R₁₅, and R₁₆ is (C₁-C₆)-alkyl, —OR₂₅, or halogen, Rₐ and R_{b} are each independently —H or —CH₃; and the remaining substituents are each hydrogen except for the R group through which the piperidine is connected. In one embodiment, R₈ is (C₁-C₆)-alkyl, —OR₂₅, halogen, —CF₃, —NO₂ or —CN and one of R₄ or R₅ is (C₁-C₆)-alkyl, —OR₂₅, halogen, or —CF₃. In one embodiment R₈ is (C₁-C₆)-alkyl, —OR₂₅, halogen, —CF₃, —NO₂ or —CN; and all other R groups are each hydrogen except for the R group through which the piperidine is connected. In one embodiment, R₈ is (C₁-C₆)-alkyl, —OR₂₅, halogen, —CF₃, —NO₂ or —CN; one of R₁₃, R₁₄, R₁₅, and R₁₆ is (C₁-C₆)-alkyl, —OR₂₅, or halogen, Rₐ and R_{b} are each independently —H or —CH₃; and the remaining substituents are each hydrogen except for the R group through which the piperidine is connected.

In one embodiment, R₇ is (C₁-C₆)-alkyl, —OR₂₅, halogen, —CF₃, —NO₂ or —CN. In one embodiment, R₇ is —OR₂₅ and R₂₅ is (C₁-C₆)-alkyl.

In one embodiment, R₁₀ is (C₁-C₆)-alkyl, —OR₂₅, halogen, —CF₃, —NO₂ or —CN. In one embodiment, R₁₀ is —OR₂₅ and R₂₅ is (C₁-C₆)-alkyl.

In one embodiment, R₁₁ is (C₁-C₆)-alkyl, —OR₂₅, halogen, —CF₃, —NO₂ or —CN. In one embodiment, R₁₁ is —OR₂₅ and R₂₅ is (C₁-C₆)-alkyl.

In one embodiment, R₁₂ is (C₁-C₆)-alkyl, —OR₂₅, halogen, —CF₃, —NO₂ or —CN. In one embodiment, R₁₂ is —CF₃.

In one embodiment, R₁, R₂, R₃, R₄, R₇, R_{7′}, R₉, R₁₀, R₁₁, and R₁₂, are each hydrogen except for the R group through which the piperidine is connected.

In one embodiment, R₁, R₂, R₃, R₄, R₇, R_{7′}, R₈, R₁₀, R₁₁, and R₁₂, are each hydrogen except for the R group through which the piperidine is connected.

In one embodiment, R₁, R₂, R₃, R₄, R₇, R_{7′}, R₈, R₉, R₁₁, and R₁₂, are each hydrogen except for the R group through which the piperidine is connected.

In one embodiment, R₁, R₂, R₃, R₄, R₇, R_{7′}, R₈, R₉, R₁₀, and R₁₁, are each hydrogen except for the R group through which the piperidine is connected.

In one embodiment, R₁, R₂, R₃, R₄, R₇, R_{7′}, R₈, and R₁₁, are each hydrogen except for the R group through which the piperidine is connected.

In one embodiment, R₁, R₂, R₃, R₄, R₇, R_{7′}, R₈, R₉, and R₁₁, are each hydrogen except for the R group through which the piperidine is connected.

In one embodiment, R₁, R₂, R₃, R₄, R₇, R_{7′}, R₈, R₉, R₁₀, R₁₁, and R₁₂, are each hydrogen except for the R group through which the piperidine is connected.

In one embodiment, R₁, R₂, R₃, R₆, R_{7′}, R₇, R₈, R₉, R₁₀, R₁₁, R₁₂, R₁₃, R₁₄, R₁₅, and R₁₆ are each hydrogen except for the R group through which the piperidine is connected.

In one embodiment, R₁, R₂, R₃, R₆, R₇, R_{7′}, R₈, R₉, R₁₀, R₁₁, and R₁₂ are each hydrogen except for the R group through which the piperidine is connected.

In one embodiment, R₁, R₂, R₃, R₄, R₅, R₆, R_{7′}, R₇, R₁₀, R₁₁, R₁₂, R₁₃, R₁₄, R₁₅, and R₁₆ are each hydrogen except for the R group through which the piperidine is connected.

In one embodiment, R₁, R₂, R₃, R₄, R₅, R₆, R_{7′}, R₇, R₁₀, R₁₁, and R₁₂ are each hydrogen.

In another embodiment, R₁₃, R₁₄, R₁₅, and R₁₆ are each hydrogen.

In one embodiment, R₃, R₆, R_{7′}, R₇, R₁₀, R₁₁, R₁₂, R₁₃, R₁₄, R₁₅, and R₁₆ are each hydrogen except for the R group through which the piperidine is connected.

In one embodiment, R₁ is —H or (C₁-C₆)-alkyl; R₄ and R₅ are each independently —H, halogen, —OR₂₅, or —CF₃; R₇, R_{7′}, R₈, R₉, R₁₀, R₁₁, and R₁₂ are each independently —H, halogen, (C₁-C₆)-alkyl, —OR₂₅, —CF₃, NO₂ or CN; R₅ is —H, halogen, or —OR₂₅; and R₃, R₆, R_{7′}, R₇, R₈, R₉, R₁₂, R₁₃, R₁₄, R₁₅, and R₁₆ are each hydrogen except for the R group through which the piperidine is connected.

In one embodiment, R₁ is —H or (C₁-C₆)-alkyl; R₂, R₈, and R₉ are each —H or F; R₄ is —H, F, —OR₂₅, or —CF₃; R₅ is —H, F, or —OR₂₅; and R₃, R₆, R₇, R₈, R₉, R₁₂, R₁₃, R₁₄, R₁₅, and R₁₆ are each hydrogen except for the R group through which the piperidine is connected.

In one embodiment, R₁ is —H or (C₁-C₆)-alkyl; R₂, R₈, and R₉ are each —H or halogen; R₄ is —H, halogen, —OR₂₅, or —CF₃; R₅ is —H, halogen, or —OR₂₅; and R₃, R₆, R_{7′}, R₇, R₈, R₉, R₁₂, R₁₃, R₁₄, R₁₅, and R₁₆ are each hydrogen except for the R group through which the piperidine is connected. In one embodiment, R₁ is —H or (C₁-C₆)-alkyl; R₂, R₈, and R₉ are each —H or F; R₄ is —H, F, —OR₂₅, or —CF₃; R₅ is —H, F, or —OR₂₅; and R₃, R₆, R₇, R₈, R₉, R₁₂, R₁₃, R₁₄, R₁₅, and R₁₆ are each hydrogen except for the R group through which the piperidine is connected.

In one embodiment, any one of R_{7′}, R₇, R₅, R₉, R₁₀, R₁₁, and R₁₂ is (C₁-C₆)-alkyl, —OR₂₅, halogen, or —CF₃, —NO₂, or —CN except for the R group through which the piperidine is connected; and any one of R₁₃, R₁₄, R₁₅, and R₁₆ is (C₁-C₆)-alkyl, —OR₂₅, halogen or —CF₃.

In one embodiment, any one of R₁, R₂, R₃, R₄, R₅, and R₆ is (C₁-C₆)-alkyl, —OR₂₅, halogen; and any one of R_{7′}, R₇, R₈, R₉, R₁₀, R₁₁, and R₁₂ is (C₁-C₆)-alkyl, —OR₂₅, halogen, or —CF₃, —NO₂, or —CN except for the R group through which the piperidine is connected.

In one embodiment, any one of R₁, R₂, R₃, R₄, R₅, and R₆ is (C₁-C₆)-alkyl, —OR₂₅, halogen; and any one of R₁₃, R₁₄, R₁₅, and R₁₆ is (C₁-C₆)-alkyl, —OR₂₅, or halogen.

In one embodiment, R₄ is (C₁-C₆)-alkyl, —OR₂₅, halogen, or —CF₃ and any one of R₁₃, R₁₄, R₁₅, and R₁₆ is (C₁-C₆)-alkyl, —OR₂₅, or halogen; and any one of R_{7′}, R₇, R₈, R₉, R₁₀, R₁₁, and R₁₂ is (C₁-C₆)-alkyl, —OR₂₅, halogen, or —CF₃, —NO₂, or —CN except for the R group through which the piperidine is connected.

In one embodiment, R₄ is (C₁-C₆)-alkyl, —OR₂₅, halogen, or —CF₃ and any one of R₁₃, R₁₄, R₁₅, and R₁₆ is (C₁-C₆)-alkyl, —OR₂₅, or halogen; and any two of R_{7′}, R₇, R₈, R₉, R₁₀, R₁₁, and R₁₂ are each independently (C₁-C₆)-alkyl, —OR₂₅, halogen, or —CF₃, —NO₂, or —CN except for the R group through which the piperidine is connected; wherein the any two of R_{7′}, R₇, R₈, R₉, R₁₀, R₁₁, and R₁₂ can be either on the same ring of the quinoline or on different rings.

In one embodiment, R₅ is (C₁-C₆)-alkyl, —OR₂₅, halogen, or —CF₃ and any one of R₁₃, R₁₄, R₁₅, and R₁₆ is (C₁-C₆)-alkyl, —OR₂₅, or halogen; and any one of R_{7′}, R₇, R₈, R₉, R₁₀, R₁₁, and R₁₂ is (C₁-C₆)-alkyl, —OR₂₅, halogen, or —CF₃, —NO₂, or —CN except for the R group through which the piperidine is connected.

In one embodiment, R₅ is (C₁-C₆)-alkyl, —OR₂₅, halogen, or —CF₃ and any one of R₁₃, R₁₄, R₁₅, and R₁₆ is (C₁-C₆)-alkyl, —OR₂₅, or halogen; and any three of R₇—, R₇, R₈, R₉, R₁₀, R₁₁, and R₁₂ are each independently (C₁-C₆)-alkyl, —OR₂₅, halogen, or —CF₃, —NO₂, or —CN except for the R group through which the piperidine is connected; wherein the any two of R_{7′}, R₇, R₈, R₉, R₁₀, R₁₁, and R₁₂ can be either on the same ring of the quinoline or on different rings.

In one embodiment, the piperidine N is connected through the R₇ of the quinoline. In another embodiment, the piperidine N is connected through the R₇ of the quinoline. In yet another embodiment, the piperidine N is connected through the R₈ of the quinoline. In still another embodiment, the piperidine N is connected through the R₉ of the quinoline.

In one embodiment, R₂₅ is (C₁-C₆)-haloalkyl.

In another embodiment, R₂₅ is (C₁-C₆)-fluoroalkyl.

In one embodiment, R₂₅ is (C₁-C₆)-alkyl. In one embodiment, R₂₅ is —CH₃.

In one embodiment, the compounds of Formula Vb are antagonists of the 5-HT_{1A} receptor. In another embodiment, the compounds of Formula Vb are agonists of the 5-HT_{1A} receptor.

In another aspect, the invention provides methods and processes of synthesizing compounds of the Formula Ve:

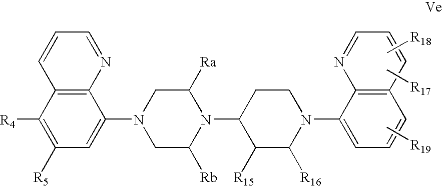

and pharmaceutically acceptable salts thereof,

wherein Rₐ, R_{b}, R₄, R₅, R₁₅, R₁₆, R₁₇, R₁₈ and R₁₉ are defined as above for Formula V; and

R₄ and R₅ cannot both be hydrogen.

In one embodiment, R₄ and R₅ are each independently —H, —OR₂₅, halogen, or (C₁-C₆)-alkyl; R₁₅ and R₁₆ are each independently —H or —CH₃; and R₁₇, R₁₈, and R₁₉ are each independently —H, —OR₂₅, halogen, (C₁-C₆)-alkyl, —CF₃, —NO₂, —CN. In one embodiment, R₄ and R₅ are each independently —H, —OCH₃, F, or —CH₃; R₁₅ and R₁₆ are each independently —H or —CH₃; and R₁₇, R₁₈, and R₁₉ are each independently —H, —OCH₃, —F, —CH₃, —CF₃, —NO₂, —CN, or —Br.

In another embodiment, R₁₉ is in the para position relative to the nitrogen of the piperidine.

In one embodiment, R₁₇ and R₁₈ are located at positions 2 and 4 of the quinoline ring (i.e., at the ortho and para positions relative to the nitrogen of the quinoline ring).

In one embodiment, R₅ is (C₁-C₆)-alkyl, —OR₂₅, halogen, or —CF₃.

In another embodiment, R₅ is (C₁-C₆)-alkyl, —OR₂₅, halogen, or —CF₃ and one of R₁₅ and R₁₆ is (C₁-C₆)-alkyl, —OR₂₅, or halogen. In a further embodiment, R₅ is (C₁-C₆)-alkyl, —OR₂₅, halogen, or —CF₃; one of R₁₅ and R₁₆ is (C₁-C₆)-alkyl, —OR₂₅, or halogen; and R₁₇, R₁₈ and R₁₉ are each hydrogen.

In yet another embodiment, R₅ is (C₁-C₆)-alkyl, —OR₂₅, halogen, or —CF₃; R₁₅ is (C₁-C₆)-alkyl, —OR₂₅, or halogen, and R₄ and R₁₆ are each hydrogen.

In one embodiment, R₅ is (C₁-C₆)-alkyl, —OR₂₅, halogen, or —CF₃; R₁₆ is (C₁-C₆)-alkyl, —OR₂₅, or halogen, and R₄ and R₁₅ are each hydrogen.

In one embodiment, R₅ is (C₁-C₆)-alkyl, —OR₂₅, halogen or —CF₃ and R₄, R₁₅, R₁₆, R₁₇, R₁₈ and R₁₉ are each hydrogen. In one embodiment, R₅ is (C₁-C₆)-alkyl, —OR₂₅, halogen, or —CF₃ and one of R₁₇, R₁₈ and R₁₉ is (C₁-C₆)-alkyl, —OR₂₅, halogen, or —CF₃. In another embodiment, R₅ is (C₁-C₆)-alkyl, —OR₂₅, halogen, or —CF₃; one of R₁₇, R₁₈ and R₁₉ is (C₁-C₆)-alkyl, —OR₂₅, halogen, or —CF₃; and the remaining substituents are each hydrogen.

In one embodiment, R₅, R₁₇, R₁₈, and R₁₉ are each independently (C₁-C₆)-alkyl, —OR₂₅, halogen or —CF₃ and R₄, R₁₅, and R₁₆ are each hydrogen. In one embodiment, R₅ is —OR₂₅ or halogen; R₁₇ and R₁₈ are each independently —OR₂₅, halogen or —CF₃; R₁₉ is halogen; and Rₐ, R_{b}, R₄, R₁₅, and R₁₆ are each hydrogen.

In one embodiment, R₄ is (C₁-C₆)-alkyl, —OR₂₅, halogen, or —CF₃.

In another embodiment, R₄ is (C₁-C₆)-alkyl, —OR₂₅, halogen, or —CF₃ and one of R₁₅, and R₁₆ is (C₁-C₆)-alkyl, —OR₂₅, or halogen.

In a further embodiment, R₄ is (C₁-C₆)-alkyl, —OR₂₅, halogen, or —CF₃; one of R₁₅ and R₁₆ is (C₁-C₆)-alkyl, —OR₂₅, or halogen; and R₁₇, R₁₈ and R₁₉ are each hydrogen.

In yet another embodiment, R₄ is (C₁-C₆)-alkyl, —OR₂₅, halogen, or —CF₃; R₁₅ is (C₁-C₆)-alkyl, —OR₂₅, or halogen, and R₅ and R₁₆ are each hydrogen.

In one embodiment, R₄ is (C₁-C₆)-alkyl, —OR₂₅, halogen, or —CF₃; R₁₆ is (C₁-C₆)-alkyl, —OR₂₅, or halogen, and R₅ and R₁₅ are each hydrogen.

In one embodiment, R₄ is (C₁-C₆)-alkyl, —OR₂₅, halogen or —CF₃ and R₅, R₁₅, R₁₆, R₁₇, R₁₈ and R₁₉ are each hydrogen.

In one embodiment, R₅ is (C₁-C₆)-alkyl, —OR₂₅, halogen, or —CF₃ and one of R₁₇, R₁₈ and R₁₉ is (C₁-C₆)-alkyl, —OR₂₅, halogen, or —CF₃.

In one embodiment, R₅ is (C₁-C₆)-alkyl, —OR₂₅, halogen, or —CF₃ and two of R₁₇, R₁₈ and R₁₉ are each independently (C₁-C₆)-alkyl, —OR₂₅, halogen, or —CF₃.

In one embodiment, R₅ is (C₁-C₆)-alkyl, —OR₂₅, halogen, or —CF₃ and three of R₁₇, R₁₈ and R₁₉ are each independently (C₁-C₆)-alkyl, —OR₂₅, halogen, or —CF₃.

In another embodiment, R₅ is (C₁-C₆)-alkyl, —OR₂₅, halogen, or —CF₃ and one of R₁₇, R₁₈ and R₁₉ is (C₁-C₆)-alkyl, —OR₂₅, halogen, or —CF₃; and the remaining substituents are each hydrogen.

In one embodiment, R₅, R₁₇, R₁₈ and R₁₉ are each independently (C₁-C₆)-alkyl, —OR₂₅, halogen, or —CF₃ and the remaining substituents are each hydrogen.

In a further embodiment, R₅ is (C₁-C₆)-alkyl, —OR₂₅, halogen, or —CF₃; one of R₁₇, R₁₈ and R₁₉ is (C₁-C₆)-alkyl, —OR₂₅, halogen, or —CF₃; one of R₁₅ and R₁₆ is (C₁-C₆)-alkyl, —OR₂₅, or halogen; and the remaining substituents are each hydrogen.

In one embodiment, R₅ is (C₁-C₆)-alkyl, —OR₂₅, halogen, or —CF₃; and any two of R₁₇, R₁₈ and R₁₉ are each independently (C₁-C₆)-alkyl, —OR₂₅, halogen, or —CF₃; and one of R₁₅, and R₁₆ is (C₁-C₆)-alkyl, —OR₂₅, halogen, or —CF₃.

In one embodiment, R₄ is (C₁-C₆)-alkyl, —OR₂₅, halogen, or —CF₃ and one of R₁₇, R₁₈ and R₁₉ is (C₁-C₆)-alkyl, —OR₂₅, halogen, or —CF₃.

In another embodiment, R₄ is (C₁-C₆)-alkyl, —OR₂₅, halogen, or —CF₃ and one of R₁₇, R₁₈ and R₁₉ is (C₁-C₆)-alkyl, —OR₂₅, halogen, or —CF₃; and the remaining substituents are each hydrogen.

In a further embodiment, R₄ is (C₁-C₆)-alkyl, —OR₂₅, halogen, or —CF₃; one of R₁₇, R₁₈ and R₁₉ is (C₁-C₆)-alkyl, —OR₂₅, halogen, or —CF₃; one of R₁₅ and R₁₆ is (C₁-C₆)-alkyl, —OR₂₅, or halogen; and the remaining substituents are each hydrogen.

In a further embodiment, R₄ is (C₁-C₆)-alkyl, —OR₂₅, halogen, or —CF₃; and any two of R₁₇, R₁₈ and R₁₉ are each independently (C₁-C₆)-alkyl, —OR₂₅, halogen, or —CF₃. In one embodiment, R₅, R₁₇, R₁₈ and R₁₉ are each independently (C₁-C₆)-alkyl, —OR₂₅, halogen, or —CF₃ and the remaining substituents are each hydrogen.

In one embodiment, one of R₁₅ and R₁₆ is —H, (C₁-C₆)-alkyl, halogen, —CF₃, or —OR₂₅. In a further embodiment, one of R₁₅ and R₁₆ is —H, (C₁-C₆)-alkyl, halogen, —CF₃, or —OR₂₅; R₅ is (C₁-C₆)-alkyl, —OR₂₅, halogen, or —CF₃; and the remaining substituents are each hydrogen. In one embodiment, R₅, R₁₇, R₁₈ and R₁₉ are each independently (C₁-C₆)-alkyl, —OR₂₅, halogen, or —CF₃ and the remaining substituents are each hydrogen.

In a further embodiment, one of R₁₅ and R₁₆ is —H, (C₁-C₆)-alkyl, halogen, —CF₃, or —OR₂₅; R₄ is (C₁-C₆)-alkyl, —OR₂₅, halogen, or —CF₃; and the remaining substituents are each hydrogen.

In one embodiment, R₄, R₁₅, R₁₆, R₁₇, R₁₈ and R₁₉ are each hydrogen.

In one embodiment, R₄, R₁₅, R₁₆, R₁₇, and R₁₈ are each hydrogen.

In one embodiment, R₄, R₁₅, and R₁₆ are each hydrogen.

In one embodiment, R₅, R₁₅, R₁₆, R₁₇, R₁₈ and R₁₉ are each hydrogen.

In one embodiment, R₅, R₁₅, R₁₆, R₁₇, and R₁₈ are each hydrogen.

In one embodiment, R₅ is —OR₂₅ or halogen; R₄, R₁₅, R₁₆, R₁₇, and R₁₈ are each hydrogen; and R₁₉ is —H or halogen.

In one embodiment, R₅ is —OCH₃ or F; R₄, R₁₅, R₁₆, R₁₇, and R₁₈ are each hydrogen; and R₁₉ is —H or F.

In one embodiment, R₅ is —OCH₃ or F; R₄, R₁₅, and R₁₆ are each hydrogen; and one of R₁₈ or R₁₉ is —H or F. In one embodiment, R₅ is —OCH₃ or F; R₄, R₁₅, R₁₆ and R₁₇ are each hydrogen; and R₁₈ and R₁₉ are each independently —CH₃ or halogen.

In one embodiment, R₅ is —OR₂₅ or halogen; R₁₇ and R₁₈ are each independently —OR₂₅, halogen or —CF₃; R₁₉ is halogen; and Rₐ, R_{b}, R₄, R₁₅, and R₁₆ are each hydrogen.

In one embodiment, R₅ is —OCH₃ or F; R₁₇ is —OCH₃; R₁₈ is —CF₃; R₁₉ is F; and Rₐ, R_{b}, R₄, R₁₅, and R₁₆ are each hydrogen.

In one embodiment, R₄ is —OR₂₅ or halogen; R₅, R₁₅, R₁₆, R₁₇ and R₁₈ are each hydrogen; and R₁₉ is —H or halogen. In one embodiment, R₅ is —OCH₃ or F; R₄, R₁₅, R₁₆ and R₁₉ are each hydrogen; and R₁₇ and R₁₈ are each —CH₃ or halogen.

In one embodiment, R₄ is —OCH₃ or F; R₅, R₁₅, R₁₆, R₁₇ and R₁₈ are each hydrogen; and R₁₉ is —H or F.

In one embodiment, R₄ is —OCH₃ or F; R₅, R₁₅, and R₁₆ are each hydrogen; and one of R₁₈ or R₁₉ is —H or F. In one embodiment, R₄ is —OCH₃ or F; R₅, R₁₅, R₁₆ and R₁₇ are each hydrogen; and R₁₈ and R₁₉ are each —CH₃ or halogen. In one embodiment, R₄ is —OCH₃ or F; R₄, R₁₅, R₁₆ and R₁₉ are each hydrogen; and R₁₇ and R₁₈ are each —CH₃ or halogen.

In one embodiment, the compounds of Formula Ve are antagonists of the 5-HT_{1A} receptor.

In another embodiment, the compounds of Formula Ve are agonists of the 5-HT_{1A} receptor.

Illustrative examples of compounds of Formula V and Formula Ve are set forth below and include, without limitation:
6-methoxy-8-[4-(1-quinolin-8-yl-piperidin-4-yl)-piperazin-1-yl]-quinoline;
6-fluoro-8-[4-(1-quinolin-8-yl-piperidin-4-yl)-piperazin-1-yl]-quinoline;
5-fluoro-8-[4-(1-quinolin-8-yl-piperidin-4-yl)-piperazin-1-yl]-quinoline;
7-fluoro-8-(4-(4-(6-methoxyquinolin-8-yl)piperazin-1-yl)piperidin-1-yl)quinoline;
6-fluoro-8-{4-[1-(8-fluoroquinolin-7-yl)piperidin-4-yl]piperazin-1-yl}quinoline;
3-trifluoromethyl-8-(4-(4-(6-methoxyquinolin-8-yl)piperazin-1-yl)piperidin-1-yl)quinoline;
6-methoxy-8-(4-(1-(quinolin-8-ylmethyl)piperidin-4-yl)piperazin-1-yl)quinoline;
5-fluoro-4-methoxy-8-(4-(4-(6-methoxyquinolin-8-yl)piperazin-1-yl)piperidin-1-yl)-2-(trifluoromethyl)quinoline;
5-fluoro-8-(4-(4-(6-methoxyquinolin-8-yl)piperazin-1-yl)piperidin-1-yl)quinoline;
8-[4-(1-quinolin-8-yl-piperidin-4-yl)-piperazin-1-yl]-quinoline;
6-chloro-8-[4-(4-(6-chloro)-quinolin-8-yl-piperidin-1-yl)-piperazin-1-yl]-quinoline;
6-fluoro-8-[4-(4-(6-chloro)-quinolin-8-yl-piperidin-1-yl)-piperazin-1-yl]-quinoline;
5-chloro-8-[4-(1-quinolin-8-yl-piperidin-4-yl)-piperazin-1-yl]-quinoline;
2-methyl-8-[4-(1-quinolin-8-yl-piperidin-4-yl)-piperazin-1-yl]-quinoline;
6-chloro-8-[4-(1-quinolin-8-yl-piperidin-4-yl)-piperazin-1-yl]-quinoline;
8-[4-(1-quinolin-8-yl-piperidin-4-yl)-piperazin-1-yl]-5-trifluoromethyl-quinoline;
5-methoxy-8-[4-(1-quinolin-8-yl-piperidin-4-yl)-piperazin-1-yl]-quinoline;
5-fluoro-8-[4-(4-quinolin-8-yl-piperazin-1-yl)-piperidin-1-yl]-quinoline;
6-methoxy-8-[4-(2-methylquinolin-8-yl-piperidin-4-yl)-piperazin-1-yl]-quinoline;
6-fluoro-8-(4-(1-(2-methylquinolin-8-yl)piperidin-4-yl)piperazin-1-yl)quinoline;
6-methoxy-8-[4-(3-methylquinolin-8-yl-piperidin-4-yl)-piperazin-1-yl]-quinoline;
6-methoxy-8-(4-(1-(4-methylquinolin-8-yl)piperidin-4-yl)piperazin-1-yl)quinoline;
6-methoxy-8-(4-(1-(2,4-dimethylquinolin-8-yl)piperidin-4-yl)piperazin-1-yl)quinoline;
6-methoxy-8-(4-(1-(2,4-dimethyl-5-fluoroquinolin-8-yl)piperidin-4-yl)piperazin-1-yl)quinoline;
6-methoxy-8-(4-(1-(2-(trifluoromethyl)quinolin-8-yl)piperidin-4-yl)piperazin-1-yl)quinoline;
6-fluoro-8-(4-(1-(5-fluoroquinolin-8-yl)piperidin-4-yl)piperazin-1-yl)quinoline;
6-methoxy-8-(4-(1-(6-bromoquinolin-8-yl)piperidin-4-yl)piperazin-1-yl)quinoline;
6-methoxy-8-(4-(1-(6-fluoroquinolin-8-yl)piperidin-4-yl)piperazin-1-yl)quinoline;
6-fluoro-8-(4-(1-(7-fluoroquinolin-8-yl)piperidin-4-yl)piperazin-1-yl)quinoline;
6-methoxy-8-{4-[1-(8-fluoroquinolin-7-yl)piperidin-4-yl]piperazin-1-yl}quinoline;
6-methoxy-8-{4-[1-(2-trifluoromethyl-4-methoxyquinolin-7-yl)piperidin-4-yl]piperazin-1-yl}quinoline;
6-methoxy-8-(4-(1-(2-trifluoromethyl-4-methoxyquinolin-8-yl)piperidin-4-yl)piperazin-1-yl)quinoline;
5-fluoro-8-(4-(4-(6-methoxyquinolin-8-yl)piperazin-1-yl)piperidin-1-yl)-2-trifluoromethylquinoline;
5-fluoro-8-(4-(4-(6-methoxyquinolin-8-yl)piperazin-1-yl)piperidin-1-yl)-3-trifluoromethylquinoline;
5-fluoro-8-(4-(4-(6-methoxyquinolin-8-yl)piperazin-1-yl)piperidin-1-yl)-4-trifluoromethylquinoline;
2,5-difluoro-8-(4-(4-(6-methoxyquinolin-8-yl)piperazin-1-yl)piperidin-1-yl)quinoline;
3,5-difluoro-8-(4-(4-(6-methoxyquinolin-8-yl)piperazin-1-yl)piperidin-1-yl)quinoline;
4,5-difluoro-8-(4-(4-(6-methoxyquinolin-8-yl)piperazin-1-yl)piperidin-1-yl)quinoline;
and pharmaceutically acceptable salts thereof.

In addition, the compounds and pharmaceutically acceptable salts of compounds of the present invention can exist as polymorphs. Such polymorphs can be transient or isolatable as a stable product. These polymorphs are within the scope of the present invention.

Prodrugs of the compounds or pharmaceutically acceptable salts of compounds are also within the scope of the present invention.

Therapeutic or Prophylactic Uses

In one embodiment, the compounds or pharmaceutically acceptable salts of the compounds of the present invention are useful as 5-HT_{1A} receptor antagonists. In another embodiment, the compounds or pharmaceutically acceptable salts of the compounds of the present invention are useful as 5-HT_{1A} receptor agonists. Accordingly, the compounds and pharmaceutically acceptable salts of the compounds of the present invention are useful for treating a mammal with a 5-HT_{1A}-related disorder. One non-limiting example of a disorder that 5-HT_{1A} receptor antagonists are useful for treating is cognition-related disorder, while a non-limiting example of a disorder that 5-HT_{1A} receptor agonists are useful for treating is anxiety-related disorder. In some embodiments, the compounds and pharmaceutical salts of the invention are useful for improving cognitive function or cognitive deficits. Examples of improvements in cognitive function include, without limitation, memory improvement and retention of learned information. Accordingly, the compounds and pharmaceutical salts of the invention are useful for slowing the loss of memory and cognition and for maintaining independent function for patients afflicted with a cognition-related disorder. Thus, in one embodiment, the compounds and pharmaceutically acceptable salts of the compounds of the present invention that act as 5-HT_{1A} receptor antagonists are useful for treating a mammal with a cognition-related disorder. In one embodiment, the compounds and pharmaceutically acceptable salts of the compounds of the present invention that act as 5-HT_{1A} receptor antagonists are useful for improving the cognitive function of a mammal. Similarly, in one embodiment, the compounds and pharmaceutically acceptable salts of the compounds of the present invention that act as 5-HT_{1A} receptor agonists are useful for treating a mammal with an anxiety-related disorder.

One nonlimiting example of a 5-HT_{1A}-related disorder is a cognition-related disorder (e.g., cognitive dysfunction). Exemplary cognition-related disorders include, without limitation, mild cognitive impairment (MCI), dementia, delirium, amnestic disorder, Alzheimer's disease, Parkinson's disease, Huntington's disease, memory disorders including memory deficits associated with depression, senile dementia, dementia of Alzheimer's disease, cognitive deficits or cognitive dysfunction associated with neurological conditions including, for example, Parkinson's disease (PD), Huntington's disease (HD), Alzheimer's disease, depression and schizophrenia (and other psychotic disorders such as paranoia and mano-depressive illness); cognitive dysfunction in schizophrenia, disorders of attention and learning such as attention deficit disorders (e.g., attention deficit hyperactivity disorder (ADHD)) and dyslexia, cognitive dysfunction associated with developmental disorders such as Down's syndrome and Fragile X syndrome, loss of executive function, loss of learned information, vascular dementia, schizophrenia, cognitive decline, neurodegenerative disorder, and other dementias, for example, due to HIV disease, head trauma, Parkinson's disease, Huntington's disease, Pick's disease, Creutzfeldt-Jakob disease, or due to multiple etiologies. Cognition-related disorders also include, without limitation, cognitive dysfunction associated with MCI and dementias such as Lewy Body, vascular, and post stroke dementias. Cognitive dysfunction associated with surgical procedures, traumatic brain injury or stroke may also be treated in accordance with the present invention.

Another nonlimiting example of a 5-HT_{1A}-related disorder is an anxiety-related disorder. Exemplary anxiety-related disorders include, without limitation, generalized anxiety disorder, attention deficit disorder, attention deficit hyperactivity disorder, obsessive compulsive disorder, substance addiction, withdrawal from drug, alcohol or nicotine addiction, panic disorder, panic attacks, post traumatic stress disorder, premenstrual dysphoric disorder, social anxiety disorder, eating disorders such as anorexia nervosa and bulimia nervosa, vasomotor flushing, and phobias, including social phobia, agoraphobia, and specific phobias. Substance addition includes, without limitation, drug, alcohol or nicotine addiction.

### EXAMPLES

### Example 1

### Preparation of 5-fluoro-8-{4-[4-(8-quinolinyl)-1-piperazinyl]-1-piperidinyl}-quinoline and Intermediates

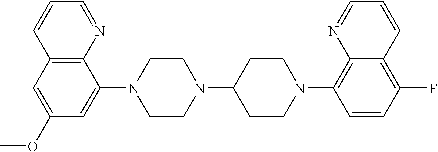

### 1. Preparation of 6-methoxy-8-(1-piperazinyl)quinoline

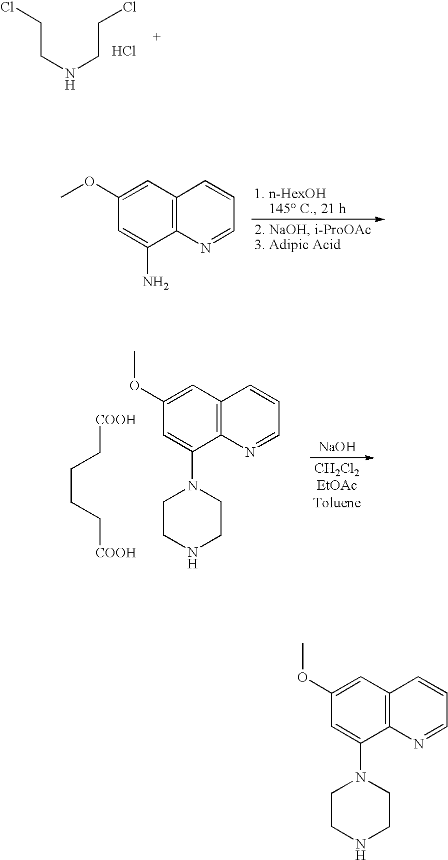

A mixture of 8-amino-6-methoxyquinoline (150.0 g, 0.862 mol) and bis(2-chloroethyl)amine (219 g, 1.23 mol) in 6 parts (volume:weight; hexanol:8-amino-6-methoxyquinoline) of 1-hexanol (900 mL) was heated to 145° C. and stirred for 21 hours. Upon completion, the reaction mixture was cooled 50-60° C., and 507 g of aqueous NaOH solution (made from 300 g of water and 207 g of 50% NaOH) was added slowly. The reaction mixture was cooled to 25-30° C. and isopropyl acetate (750 mL) was added.

The mixture was then clarified through a celite pad. The aqueous phase was subsequently split off, and discarded. The organic solution was treated with a slurry of adipic acid (126 g, 0.862 mol) in isopropyl acetate (250 ml). The resulting mixture was stirred for 16 hours to form 6-methoxy-8-(1-piperazinyl)quinoline adipate salt. The adipate salt was filtered and washed with isopropyl acetate (2×150 ml) and dried by nitrogen flow to give adipate of 6-Methoxy-8-piperazin-1-yl-quinoline (186 g, 55% yield) with ˜97% HPLC area, 88% strength purity in 51% yield.

The salt was recrystallized from a mixture of methanol and isopropyl acetate. This was done due to the need for further purification. However, if purification is not required, the following procedure can be eliminated.

To purify the adipate salt, 580 g of the crude adipate salt and 2.8 liter of methanol were mixed and heated to 65° C. and a dark solution was obtained. To this solution was charged slowly 1.1 liter of isopropyl acetate over 40 min at about 63° C. The mixture was stirred at about 63° C. for about 1 h and cooled to 0-5° C. After stirring at 0-5° C. for 2 hours, the mixture was filtered and washed with 300 ml of isopropyl acetate and dried with airflow. The total yield was 395 g, or 68.1% recovery.

To liberate 6-methoxy-8-(1-piperazinyl)quinoline from its adipate salt, 100 g (0.257 mol) of the adipate salt was added into a 2-L reactor followed by the addition of 500 ml of dichloromethane. To this mixture was added 100 g of water followed by the slow (in about 15 min) addition of 41 g of 50% sodium hydroxide solution to maintain the pH in the 13-14 range (additional sodium hydroxide solution may be needed if the pH is below 10). The organic bottom layer was separated and filtered through a pad of activated basic aluminum oxide (100 g, 6.5 cm diameter×3 cm depth). The pad was washed with 100 ml of isopropyl acetate twice. The dichloromethane was replaced by toluene by distillation under vacuum (450 to 500 mm Hg) while 3×150 ml of toluene was added into the reactor until the final volume was about 135 ml. This solution was used for reductive amination.

White solid precipitated after distillation. The solid was removed by filtration, and the resultant filter cake was washed with 50 ml of toluene. The final volume was 185 ml at a purity of 97.56%, and a solution strength of 27.4%.

### 2. Preparation of 8-bromo-5-fluoroquinoline via 2-bromo-5-fluoroaniline Intermediate

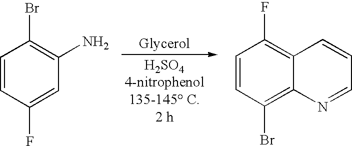

To a 2-L reactor equipped with a mechanic agitator, a condenser, a thermocouple, a baffle, and nitrogen inlet were charged with an aqueous solution of sulfuric acid made from 267 ml concentrated sulfuric acid and 114 mL of water. The sulfuric acid was heated to 140-150° C. Prior to addition to the hot sulfuric acid, 228 g of water, 200 g of 2-bromo-5-fluoroaniline, 97 g of glycerol, and 80 g of 4-nitrophenol were mixed at between 25-50° C. The 2-bromo-5-fluoroaniline mixture was added slowly over 1.5 hours to the diluted, hot (140-150° C.) sulfuric acid. The mixture was incubated at 135-145° C. for 1 hour after the addition. The reaction mixture was cooled to below 20-50° C., and the reaction mixture was transferred slowly to a 5-L reactor containing 1100 g of water and 1210 g of toluene.

The 2-L reactor was washed with 300 g of water and the wash was combined into the 5-L reactor. The pH of the contents in the 5-L reactor was adjusted to pH 8-10 by adding approximately 1233 g (1370 mL) ammonium hydroxide (28-30% NH3) at 20-40° C. The mixture was stirred at room temperature for 15 min and the solid by-product was filtered off while the filtrate was retained. The filter cake was washed with 400 ml of toluene and the all the filtrate was combined and charged a 3-L reactor. About 500 ml of 8.5% KOH solution was charged into the 3-L reactor and stirred for 10 min and bottom aqueous layer was split off. A second portion of 500 ml of 8.5% KOH solution was added and the mixture was stirred for 15 min and the bottom aqueous layer was split off. Water, at a volume of 500 ml, was added and stirred for 15 min before the bottom aqueous layer was split off. The aqueous layers were subsequently discarded. The organic layer was heated to distill off about 100-200 ml of toluene to azeotropically remove water. A clear solution was obtained. This solution was used directly in the following step.

During the typical reaction scheme, the yield is 178 g real 8-bromo-5-fluoroquinoline, ˜75%. The yield for the above reaction scheme was 87.5% at a product purity of over 99%.

### 3. Preparation of 1-(5-Fluoroquinolin-8-yl)piperidin-4-one

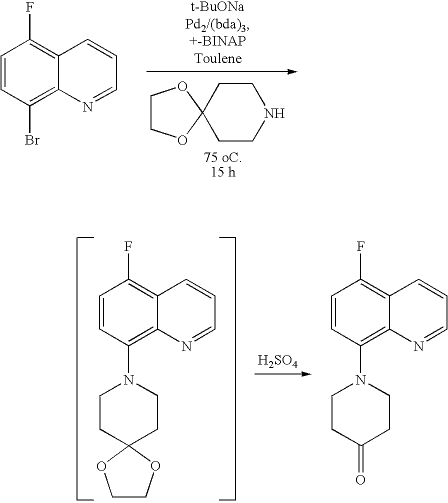

To a 5-L jacketed cylindrical reactor equipped with an impeller-style agitator, condenser, thermocouple, and vacuum/nitrogen inlet was charged 2-L, 15% toluene solution of 8-bromo-5-fluoroquinoline produced in the step above, 209 g of 1,4-Dioxa-8-azaspiro[4.5]decane. Meanwhile in a 500-mL Erlenmeyer flask, a suspension of 16.5 g (26.5 mmol)±-[1,1′-binaphthalene]-2,2′-diylbis[diphenyl-Phosphine, and 6.08 g (6.64 mmol) tris[m-[(1,2-h:4,5-h)-(1E,4E)-1,5-diphenyl-1,4-pentadien-3-one]]dipalladium in 260 g of toluene was prepared. This freshly made suspension was charged into the 5-L reactor followed by a rinse of 170 g of toluene. 166 g sodium tert-butoxide was then charged into the reactor followed by a rinse with 430 g of toluene. The reactor was degassed by vacuum to less than 125 mmHg and then filled with nitrogen to atmosphere three times. The mixture was then heated to 50-60° C. and stirred for 1 hour and then heated to 65-75° C. and stirred at this temperature for about 10 hours.

The mixture was subsequently cooled to 40-50° C. and then quenched with 800 g of water. The lower aqueous layer was split off and the volume of the organic layer was reduced to about 1.5 L by vacuum distillation. To this residual was charged 2.28 kg of 20% sulfuric acid at 25-30° C. The mixture was stirred for an hour and was clarified by filtration and a bi-phase filtrate was obtained. The aqueous phase was split off and retained. Toluene (870 g) was added to the aqueous solution and the mixture was neutralized by slowly adding 770 g of a 50% sodium hydroxide solution.

The lower aqueous layer was split off and extracted with 600 g of toluene. The organic layers were combined and the volume of the reaction was reduced to about 1 L by vacuum distillation. The residue was cooled to room temperature and 480 g of toluene was charged. The mixture was heated to 45-55° C. to form a clear solution, which was filtered through a celite/charcoal pad to remove palladium. The filtrate was concentrated by vacuum distillation to about 0.7 L and diluted with 620 g heptane, cooled to −15 to −5° C. to form a slurry. The solid was collected by filtration. The product was dried by air-flow at room temperature.

The overall yield was about 70%.

### 4. Preparation of 5-Fluoro-8-(4-(4-(6-methoxyquinolin-8-yl)piperazin-1-yl)piperidin-1-yl)-quinoline

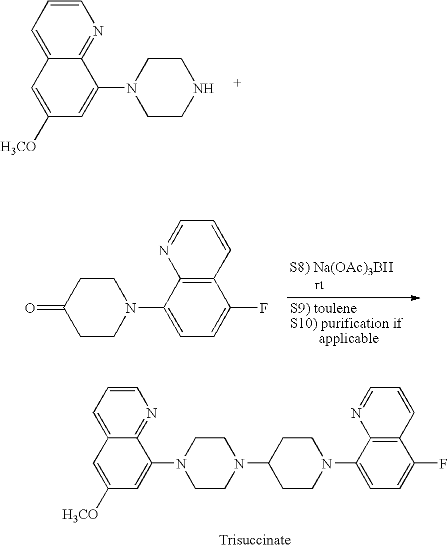

Toluene (118 g), sodium triacetoxyborohydride (44.5 g) were mixed at 0° C. to room temperature. To this mixture was charged a premixed toluene solution of 160 g, or 27.4 wt % in toluene, of 6-methoxy-8-(1-piperazinyl)quinoline and 41 g of 1-(5-Fluoroquinolin-8-yl)piperidin-4-one. The resulting mixture was stirred for 2 to 3 hours at about 30° C. KOH solution (443 g 9% in water) was charged to quench the residual sodium triacetoxyborohydride. Heptane (118 g) was added to further precipitate the product. The product was then filtered and washed with ethanol (2×100 ml). The yield was 68 g, or approximately 86%.

This crude product (67 g) was dissolved in 586 g dichloromethane and passed through a charcoal/celite pad to remove palladium. The dichloromethane was distilled off while 400 g of ethanol was slowly added at the same time. The resulting slurry was filtered and washed with ethanol twice (65 g+100 g). The product was dried in oven at 55° C. overnight. The recovery yield for the purification procedure was 59.9 g, or approximately 89.4%.

Production of the trisuccinate salt of 5-Fluoro-8-(4-(4-(6-methoxyquinolin-8-yl)piperazin-1-yl)piperidin-1-yl)-quinoline was accomplished as follows. Briefly, 55 g (0.127 mol) of 5-Fluoro-8-(4-(4-(6-methoxyquinolin-8-yl)piperazin-1-yl)piperidin-1-yl)-quinoline was dissolved in 440 ml of dichloromethane. This solution was charged in 20 min into a 3-L reactor containing 42.7 g (0.361 mol) succinic acid and 1.5 L acetone at 30-35° C. The product crystallized out of the solution and then dichloromethane was distilled off while simultaneously 1.5 kg of 2-butanone was added. The resulting slurry was filtered and the crystalline solid was collected.

The yield was 74.0 g at approximately 77%.

### Example 2

### Alternative Preparation of 5-fluoro-8-{4-[4-(8-quinolinyl)-1-piperazinyl]-1-piperidinyl}-quinoline and Intermediates

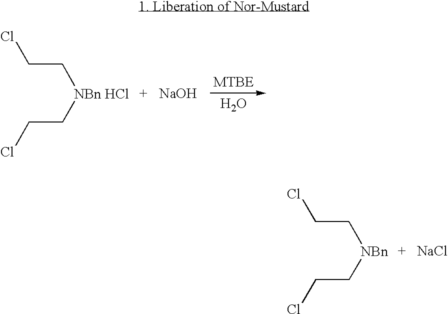

To a 2 L reactor equipped with a mechanic stirrer, an addition funnel, a thermocouple, nitrogen inlet and a bottom outlet was added 442 g of water, 134.5 g (0.5 mol) nor-mustard and 177 g methyl tert-butyl ether. To this mixture was slowly added 125 ml, 5 N, sodium hydroxide over a period of 20 min. The mixture was stirred for 10 min and the aqueous layer was split off. The organic layer was washed with 20% aqueous sodium chloride solution twice (2×200 g). The methyl tert-butyl ether was distilled off and the product was obtained as an oil. The yield was 117 g at ˜100% yield. The product still contained trace amount solvent.

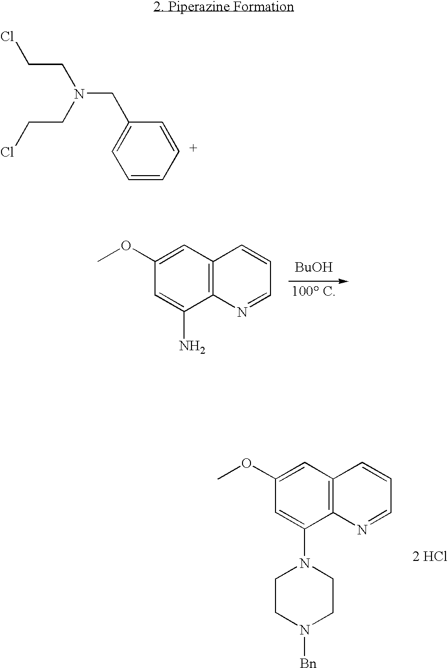

To a 1-L reactor a mechanic stirrer, a thermocouple, and nitrogen inlet was charged 380 g of butanol, 42 g of 8-amino-6-methoxyquinoline and 97 g of nor-mustard free amine. The mixture was heated to 100° C. for 18 hours before cooled to 0-5° C. A solid was formed upon cooling and was filled with nitrogen protection. The solid was hygroscopic. The filter cake was washed with 100 g cold butanol and 2×200 g of MTBE. The solid was dissolved in 160 g of water to obtain an orange solution.

This orange solution was slowly charged into a 2-L reactor containing a potassium hydroxide solution prepared with 537 g water and 60 g 45% KOH. The product was precipitated upon addition into the base. The slurry was stirred for 1 hour and then filtered. The filter cake was washed with 100 g water, 100 g MeOH and 100 g methyl tert-butyl ether. The product was dried under vacuum at 50° C. Weight=48.2 g, 60%

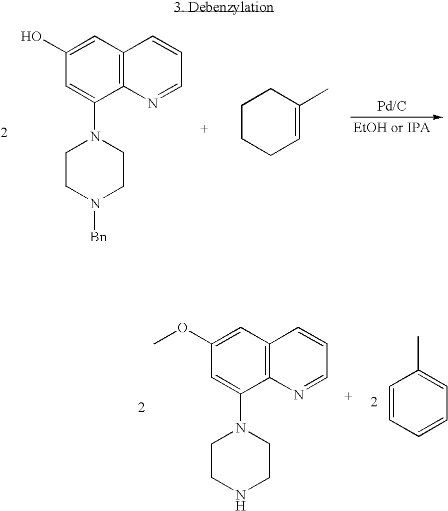

To a 100 ml flask equipped with a stirrer, a thermocouple, a condenser and nitrogen inlet was charged ethanol 27 g, 8-(4-Benzyl-piperazin-1-yl)-6-methoxy-quinoline (2 g), methylcyclohexene (10 g), and 0.6 g of dry 10% palladium on carbon. The mixture was heated to reflux for 30 hours, and cooled to ambient temperature. The palladium on carbon was filtered off, and the solvent was removed by rotavap. The weight of the product yield was 1.7 g. The product contained small amounts of solvent.

### Example 3

### Preparation of Disuccinate Salt of 5-Fluoro-4-methoxy-8-(4-(4-(6-methoxy-quinolin-8-yl)-piperazin-1-yl)-piperidin-1-yl)-2-trifluoromethyl-quinoline

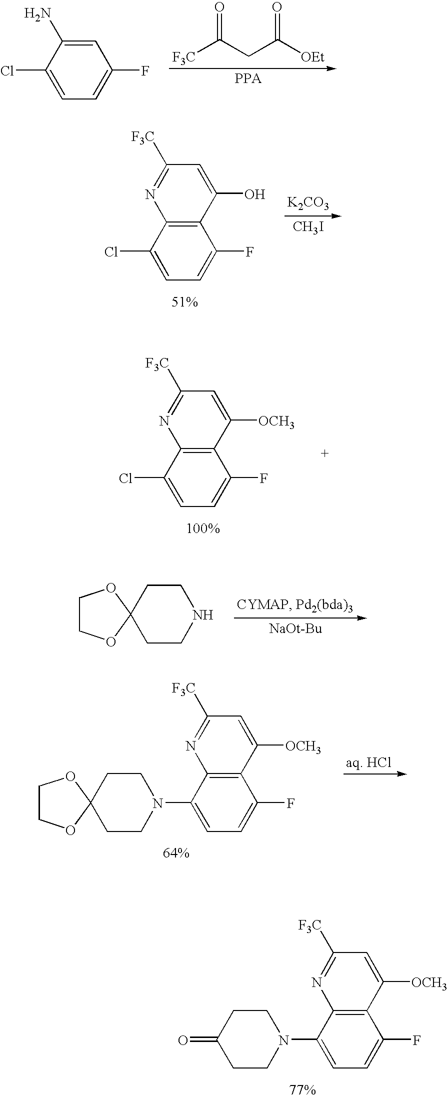

### 1. 8-Chloro-5-fluoro-2-(trifluoromethyl)quinolin-4-ol

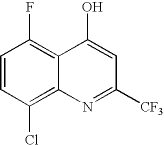

A solution of ethyl 4,4,4-trifluoroacetoacetate (commercially available, 4 mL, 27.3 mmol, 1.05 eq.) in polyphosphoric acid (22 mL) was heated to 100° C. 2-chloro-5-fluoroaniline (3.78 g, 26.0 mmol, 1 eq.) was added slowly to the stirred hot solution. The resulting reaction mixture was further heated to 150° C. and then stirred at that temperature overnight (approximately 18 hours). The reaction was cooled to room temperature and water was added carefully. The resulting light brown precipitate was collected by vacuum filtration, washed with water and dissolved in ethyl acetate. The ethyl acetate solution was washed with brine, dried over anhydrous MgSO4 and concentrated on a rotary evaporator. The crude product was purified by flash chromatography on silica gel using hexane/ethyl acetate to give 3.54 g (51% yield) of the desired product as an off-white solid; MP=141-142; MS (ES) m/z (relative intensity): 266 (M+H)+ (100).

### 2. 8-Chloro-5-fluoro-4-methoxy-2-(trifluoromethyl)quinoline

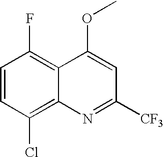

To a solution of 8-Chloro-5-fluoro-2-(trifluoromethyl)quinolin-4-ol (Step 1, 3.54 g, 13.3 mmol, 1 eq.) in acetone (75 mL) was added anhydrous K₂CO₃ (3.88 g, 28.0 mmol, 2.1 eq.), followed by iodomethane (1.8 mL, 28.9 mmol, 2.17 eq.). The resulting mixture was stirred at reflux for 1.5 hours. An additional aliquot of iodomethane (1.8 mL, 28.9 mmol, 2.17 eq.) was added and reflux was continued for an additional 1 hour. The reaction was cooled to room temperature, poured onto ice and extracted with ethyl acetate. The combined organic layers were dried over anhydrous MgSO₄, filtered and concentrated on a rotary evaporator to give 3.72 g (100% yield) of the desired product as a yellow solid, which was used in subsequent reactions without further purification. An analytical sample was prepared by recrystallization from hexane/ethyl acetate; MP=198-200° C.; MS (ES) m/z (relative intensity): 280 (M+H)⁺ (100).

### 3. 8-(1,4-Dioxa-8-azaspiro[4,5]dec-8-yl)-5-fluoro-4-methoxy-2-(trifluoromethyl)-quinoline

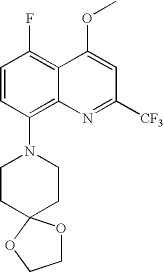

To a solution of 8-chloro-5-fluoro-4-methoxy-2-(trifluoromethyl)quinoline (Step 2, 1.24 g, 4.45 mmol, 1 eq.) in anhydrous tetrahydrofuran (44 mL) was added tris(dibenzylideneacetone)-dipalladium(0) (Pd₂(dba)₃, 0.125 g, 0.14 mmol, 0.03 eq.), sodium tert-butoxide (0.69 g, 7.18 mmol, 1.61 eq.), 2-dicyclohexyl-phosphino-2′-(N,N-dimethylamino)biphenyl (CYMAP, 0.054 g, 0.14 mmol, 0.03 eq.), and 1,4-dioxo-8-azaspiro-4,5-decane (0.8 mL, 6.24 mmol, 1.4 eq.). The resulting mixture was stirred at 70° C. overnight (approximately 18 hours) under a nitrogen atmosphere. The reaction was then cooled to room temperature, diluted with ether, filtered through a plug of silica gel and concentrated on a rotary evaporator. The crude product was purified by flash chromatography on silica gel using hexane/ethyl acetate to give 1.09 g (64% yield) of the desired product as a beige solid; MP=101-103° C.; MS (ES) m/z (relative intensity): 387 (M+H)⁺ (100).

### 4. 1-[5-Fluoro-4-methoxy-2-(trifluoromethyl)quinolin-8-yl]piperidin-4-one

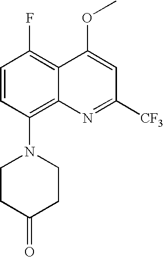

To a solution of 8-(1,4-dioxa-8-azaspiro[4,5]dec-8-yl)-5-fluoro-4-methoxy-2-(trifluoro-methyl)quinoline (Step 3, 0.6 g, 1.56 mmol, 1 eq.) in tetrahydrofuran (20 mL) was added 2N aqueous HCl (6 mL). The resulting mixture was stirred at 70° C. for 5 hours. The reaction was cooled to room temperature, poured into 1N aqueous sodium hydroxide and extracted with ethyl acetate. The combined organic layers were dried over anhydrous Na₂SO₄, and concentrated on a rotary evaporator. The crude product was purified by flash chromatography on silica gel using hexane/ethyl acetate to give 0.41 g of the desired product as a light yellow solid; MP=171-173C; MS (ES) m/z (relative intensity): 343 (M+H)⁺ (100).

5. 5-fluoro-4-methoxy-8-(4-(4-(6-methoxyquinolin-8-yl)piperazin-1-yl)piperidin-1-yl)-2-(trifluoromethyl)quinoline trihydrochloride

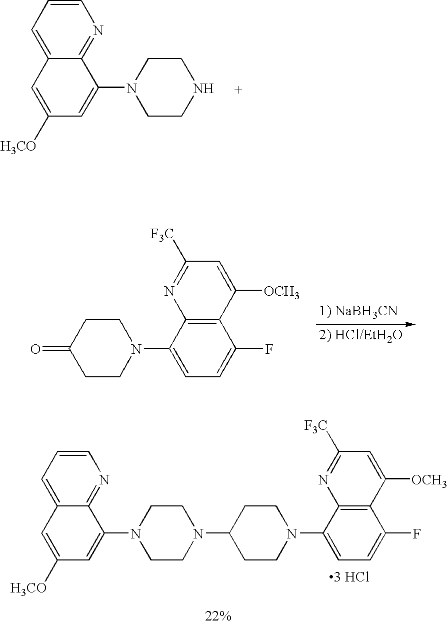

To a solution of 1-[5-fluoro-4-methoxy-2-(trifluoromethyl)quinolin-8-yl]piperidin-4-one (Step 4, 0.31 g, 0.9 mmol, 1 eq.) and 6-methoxy-8-(1-piperazinyl)quinoline (Example A, Step 4, 0.30 g, 1.23 mmol, 1.37 eq.) in anhydrous methanol (20 mL) was added sodium cyanoborohydride (0.103 g, 1.64 mmol, 1.82 eq.). The resulting mixture was stirred overnight at room temperature under nitrogen (app. 18 hr). An additional aliquot of sodium cyanoborohydride (0.10 g, 1.59 mmol, 1.76 eq.) was added and stirring at room temperature was continued overnight. The resulting reaction mixture was poured into brine and extracted with ethyl acetate. The organic layer was dried over anhydrous sodium sulfate and concentrated on a rotary evaporator to a yellow oil. The desired product was isolated by chromatography on a 40 g silica column (1000 mL 20% acetone in hexane followed by 500 mL 30% acetone in hexane) as a yellow solid (0.113 g, 22% yield). The free base was converted to its trihydrochloride sesquihydrate salt by dissolving it in dichloromethane (3 mL), adding diethyl ether (9 mL), cooling in an ice bath and adding 1M HCl/Et20 (1 mL). The resulting yellow solid was collected by vacuum filtration, washed with ether and dried in vacuo to give 0.152 g. MS (ES) m/z (relative intensity): 570 (M+H)+ (100).

### 6. Synthesis of Disuccinate Salt of 5-Fluoro-4-methoxy-8-(4-(4-(6-methoxy-quinolin-8-yl)-piperazin-1-yl)-piperidin-1-yl)-2-trifluoromethyl-quinoline

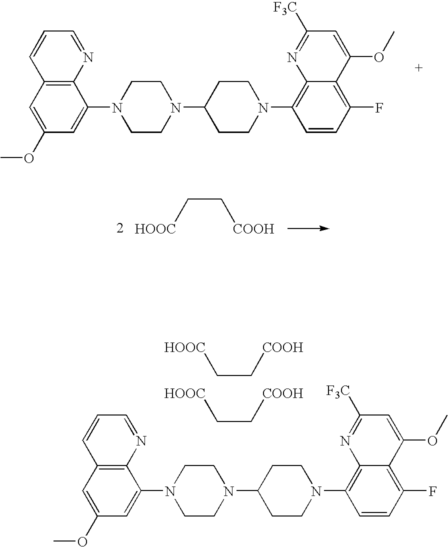

The free base of the compound synthesized in Part 5 of this Example was isolated as a disuccinic acid. To a 12-L reactor equipped with heating mantle, thermocouple and nitrogen inlet were charged 124 g of succinic acid and 2470 g of acetone. The mixture was heated to 50° C. and a colorless solution was formed. Meanwhile, in a 3-L flask were charged 240 g of 5-Fluoro-4-methoxy-8-(4-(4-(6-methoxy-quinolin-8-yl)-piperazin-1-yl)-piperidin-1-yl)-2-trifluoromethyl-quinoline and 2250 g of THF. 5-Fluoro-4-methoxy-8-(4-(4-(6-methoxy-quinolin-8-yl)-piperazin-1-yl)-piperidin-1-yl)-2-trifluoromethyl-quinoline in THF mixture was heated to 50° C. and a yellow solution was achieved. This yellow solution was slowly (about 3 hours) charged into the 12-L reactor while maintain both solution temperature at about 50° C. The resulting slurry was stirred over night at room temperature, and then cooled to 5-10° C. After stirred at 5-10° C. for 2 hours, the slurry was filtered and the product was washed with acetone 3×600 ml. The product was dried with airflow at room temperature for 3 hours.

The weight of the product was 311 g, or about 91.6% yield. NMR analysis indicated that the compound was the disuccinate salt form of 5-Fluoro-4-methoxy-8-(4-(4-(6-methoxy-quinolin-8-yl)-piperazin-1-yl)-piperidin-1-yl)-2-trifluoromethyl-quinoline. In addition, residual solvents were found at concentrations of 0.047% for acetone, 0.027% for THF, and 0.14% for water.

What is claimed is:

1. A process for isolating a compound having Formula I: 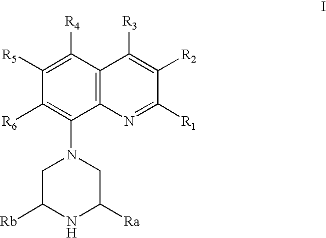
or a pharmaceutically acceptable salt thereof,
wherein R₁, R₂, R₃, R₄, R₅, and R₆, are each independently —H, (C₁-C₆)-alkyl, (C₁-C₆)-haloalkyl, (C₂-C₆)-alkenyl, or (C₂-C₆)-alkynyl, halogen, —CF₃, —NO₂, —CN, —OR₂₅, —OSO₂R₂₅, —SR₂₅, —SO₂R₂₅, —SO₂N(R₂₅)₂, —N(R₂₅)₂, C(O), —COR₂₅, —CO₂R₂₅, —NR₂₅CO₂R₂₅, —NR₂₅COR₂₅, —NR₂₅CON(R₂₅)₂, or —CON(R₂₅)₂;
Ra and Rb are each independently —H or —CH₃; and
R₂₅ is —H; or linear or branched (C₁-C₆)-alkyl, (C₁-C₆)-haloalkyl, (C₂-C₆)-alkenyl, or (C₂-C₆)-alkynyl, the process comprising:
(a) reacting a compound having Formula I with a dicarboxylic acid to form an addition salt of the compound having Formula I:
(b) isolating the compound of Formula I from the addition salt of Formula I in the presence of an organic solvent, a base, and CH₂Cl₂.

2. The process of claim 1, wherein the dicarboxylic acid is a (C₃-C₁₂)-alkyl dicarboxylic acid.

3. The process of claim 2, wherein the (C₃-C₁₂)-alkyl dicarboxylic acid is adipic acid.

4. The process of claim 1, wherein R₁, R₂, R₃, R₄, R₅, and R₆, are each independently —H, (C₁-C₆)-alkyl, (C₁-C₆)-haloalkyl, (C₂-C₆)-alkenyl, or (C₂-C₆)-alkynyl, halogen, —CF₃, —NO₂, —CN, or —OR₂₅.

5. The process of claim 1, wherein R₅ is —OR₂₅, and R₂₅ is (C₁-C₆)-alkyl.

6. The process of claim 1, wherein R₅ is a methoxy.

7. The process of claim 1, wherein the organic solvent is toluene.

8. The process of claim 1, wherein the base is NaOH or KOH.

9. A process for synthesizing a compound comprising:
a) mixing an optionally substituted aniline compound having Formula II with and glycerol and 4-nitrophenol to form a first solution solution: 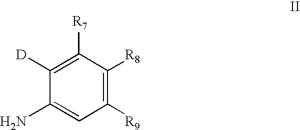
wherein D is halogen, and
wherein R₇, R₈, and R₉, are each independently —H, (C₁-C₆)-alkyl, (C₁-C₆)-haloalkyl, (C₂-C₆)-alkenyl, or (C₂-C₆)-alkynyl, halogen, —CF₃, —NO₂, —CN, —OR₂₅, —OSO₂R₂₅, —SR₂₅, —SO₂R₂₅, —SO₂N(R₂₅)₂, —N(R₂₅)₂, C(O), —COR₂₅, —CO₂R₂₅, —NR₂₅CO₂R₂₅, —NR₂₅COR₂₅, —NR₂₅CON(R₂₅)₂, or —CON(R₂₅)₂; and R₂₅ is —H; or linear or branched (C₁-C₆)-alkyl, (C₁-C₆)-haloalkyl, (C₂-C₆)-alkenyl, or (C₂-C₆)-alkynyl;
b) reacting the first solution with an acid to form a compound of Formula III: 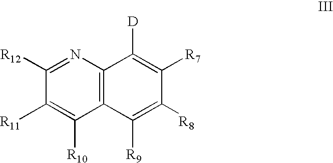
wherein D is halogen, and wherein R₇, R₈, R₉, R₁₀, R₁₁, and R₁₂, are each independently —H, (C₁-C₆)-alkyl, (C₁-C₆)-haloalkyl, (C₂-C₆)-alkenyl, or (C₂-C₆)-alkynyl, halogen, —CF₃, —NO₂, —CN, —OR₂₅, —OSO₂R₂₅, —SR₂₅, —SO₂R₂₅, —SO₂N(R₂₅)₂, —N(R₂₅)₂, C(O), —COR₂₅, —CO₂R₂₅, —NR₂₅CO₂R₂₅, —NR₂₅COR₂₅, —NR₂₅CON(R₂₅)₂, or —CON(R₂₅)₂, and R₂₅ is —H; or linear or branched (C₁-C₆)-alkyl, (C₁-C₆)-haloalkyl, (C₂-C₆)-alkenyl, or (C₂-C₆)-alkynyl,
wherein the method comprises mixing the optionally substituted aniline compound having Formula II, glycerol, and 4-nitrophenol to form a solution prior to addition of acid.

10. The process of claim 9, wherein the acid is H₂SO₄.

11. The process of claim 9, wherein the temperature of the acid is greater than about 50° C.

12. The process of claim 9, wherein the temperature of the acid is greater than about 100° C.

13. The process of claim 9, wherein the temperature of the acid is greater than about 120° C.

14. The process of claim 9, wherein the temperature of the acid is between about 135° C. and about 145° C.

15. The process of claim 9, wherein the temperature of the acid is less than about 150° C.

16. The process of claim 9, wherein D is bromine or chlorine.

17. The process of claim 16, wherein D is bromine.

18. The process of claim 9, wherein R₉ is halogen.

19. The process of claim 18, wherein R₉ is fluorine.

20. The process of claim 9, wherein R₇ and R₈ are each independently hydrogen.

21. The process of claim 9, wherein R₁₁ is hydrogen.

22. The process of claim 9, wherein R₁₂ is hydrogen.

23. The process of claim 9, wherein R₉ is halogen, R₁₁ is hydrogen, and R₁₂ is hydrogen.

24. The process of claim 23, wherein D is bromine.

25. The process of claim 9 further comprising heating a mixture of the solution and H₂SO₄ to between 135° C. and 140° C.

26. A process of synthesizing a compound comprising:
a) reacting an optionally substituted piperazino-quinoline compound of Formula I: 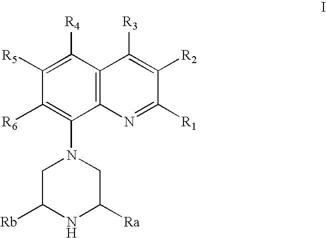
wherein R₁, R₂, R₃, R₄, R₅, and R₆, are each independently —H, (C₁-C₆)-alkyl, (C₁-C₆)-haloalkyl, (C₂-C₆)-alkenyl, or (C₂-C₆)-alkynyl, halogen, —CF₃, —NO₂, —CN, —OR₂₅, —OSO₂R₂₅, —SR₂₅, —SO₂R₂₅, —SO₂N(R₂₅)₂, —N(R₂₅)₂, C(O), —COR₂₅, —CO₂R₂₅, —NR₂₅CO₂R₂₅, —NR₂₅COR₂₅, —NR₂₅CON(R₂₅)₂, or —CON(R₂₅)₂;
Ra and Rb are each independently —H or —CH₃; and
R₂₅ is —H; or linear or branched (C₁-C₆)-alkyl, (C₁-C₆)-haloalkyl, (C₂-C₆)-alkenyl, or (C₂-C₆)-alkynyl;
with an optionally substituted piperidin-4-one compound of Formula IV: 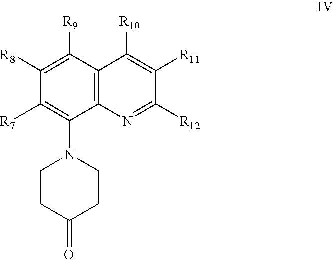
wherein R₇, R₈, R₉, R₁₀, R₁₁, and R₁₂, are each independently —H, (C₁-C₆)-alkyl, (C₁-C₆)-haloalkyl, (C₂-C₆)-alkenyl, or (C₂-C₆)-alkynyl, halogen, —CF₃, —NO₂, —CN, —OR₂₅, —OSO₂R₂₅, —SR₂₅, —SO₂R₂₅, —SO₂N(R₂₅)₂, —N(R₂₅)₂, C(O), —COR₂₅, —CO₂R₂₅, —NR₂₅CO₂R₂₅, —NR₂₅COR₂₅, —NR₂₅CON(R₂₅)₂, or —CON(R₂₅)₂, and R₂₅ is —H; or linear or branched (C₁-C₆)-alkyl, (C₁-C₆)-haloalkyl, (C₂-C₆)-alkenyl, or (C₂-C₆)-alkynyl;
in the presence of toluene and under conditions effective to bring about reductive amination at the piperidine carbonyl, thereby providing an piperazine-piperidine compound having the Formula V:

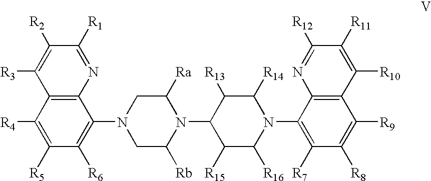

27. The process of claim 26, wherein R₅ is —H, (C₁-C₆)-alkyl, OR₂₅, halogen, or CF₃.

28. The process of claim 26, wherein R₉ is —H, (C₁-C₆)-alkyl, OR₂₅, halogen, CF₃, —NO₂, or —CN.

29. The process of claim 26, wherein R₁₀ is —H, (C₁-C₆)-alkyl, OR₂₅, halogen, CF₃, —NO₂, or —CN.

30. The process of claim 26, wherein R₁₂ is —H, (C₁-C₆)-alkyl, OR₂₅, halogen, CF₃, —NO₂, or —CN.

31. The process of claim 26, wherein R₅ is —H, (C₁-C₆)-alkyl, OR₂₅, halogen, or CF₃; and R₉ is —H, (C1-C6)-alkyl, OR₂₅, halogen, CF₃, —NO₂, or —CN.

32. The process of claim 26, wherein R₅ is —H, (C1-C6)-alkyl, OR₂₅, halogen, or CF₃ and one of R₇, R₅, R₉, R₁₀, R₁₁, and R₁₂ is —H, (C₁-C₆)-alkyl, OR₂₅, halogen, CF₃, —NO₂, or —CN.

33. The process of claim 26, wherein any one of R₁, R₂, R₃, R₄, R₅, and R₆ is —H, (C₁-C₆)-alkyl, OR₂₅, halogen, or CF₃; and any three of R₇, R₈, R₉, R₁₀, R₁₁, and R₁₂ is —H, (C₁-C₆)-alkyl, OR₂₅, halogen, CF₃, —NO₂, or —CN.

34. The process of claim 26, wherein R₂₅ is (C1-C6)-alkyl.

35. The process of claim 26, wherein n is 1.

36. The process of claim 26, wherein R₅ is —OR₂₅, R₂₅ being linear or branched (C₁-C₆)-alkyl and R₁, R₂, R₃, R₄, and R₆ are each —H.

37. The process of claim 36, wherein R₅ is methoxy.

38. The process of claim 37, wherein RF and R_{b} are each independently hydrogen.

39. The process of claim 38, wherein R₉ is halogen, and R₇, R₈, R₁₀, R₁₁, and R₁₂, are each hydrogen.

40. The process of claim 39, wherein R₉ is halogen.

41. The process of claim 26 further comprising reacting the compound having Formula IV and the compound of Formula V in the presence of a compound having the Formula VI:

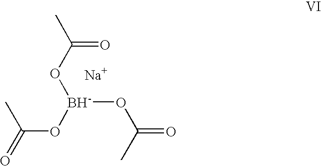

42. The process of claim 41 further comprising
b) premixing toluene and the compound having Formula VII to form a first organic solution;
c) premixing the compound having Formula IV with the compound having Formula V in toluene to form a second organic solution; and
(d) mixing the first and second organic solutions under conditions effective for reacting Formula IV and Formula V to produce Formula VI.

43. A process for isolating a compound comprising:
a) mixing a compound of Formula VII with a first organic solvent to make a first solution: 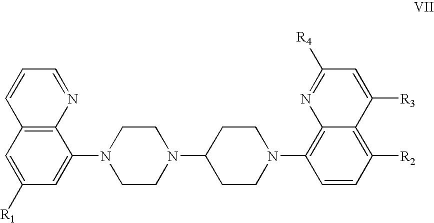
wherein R₁, R₂, R₃, and R₄, are each independently —H, (C₁-C₆)-alkyl, (C₁-C₆)-haloalkyl, (C₂-C₆)-alkenyl, or (C₂-C₆)-alkynyl, halogen, —CF₃, —NO₂, —CN, —OR₂₅, —OSO₂R₂₅, —SR₂₅, —SO₂R₂₅, —SO₂N(R₂₅)₂, —N(R₂₅)₂, C(O), —COR₂₅, —CO₂R₂₅, —NR₂₅CO₂R₂₅, —NR₂₅COR₂₅, —NR₂₅CON(R₂₅)₂, or —CON(R₂₅)₂; and
R₂₅ is —H; or linear or branched (C₁-C₆)-alkyl, (C₁-C₆)-haloalkyl, (C₂-C₆)-alkenyl, or (C₂-C₆)-alkynyl;
b) mixing the first solution with a second solution, the second solution comprising a second organic solvent and a dicarboxylic acid; and
c) isolating the dicarboxylic acid addition salt of the compound of Formula VII from the mixture,
wherein the isolated dicarboxylic acid addition salt of the compound of Formula VII contains less than 0.25 w % of each solvent used during the synthesis of the compound of Formula VII.

44. The process of claim 43, wherein dicarboxylic acid is succinic acid.

45. The process of claim 43, wherein the first organic solvent is THF.

46. The process of claim 43, wherein the second organic solvent is acetone.

47. The process of claim 46, wherein the first organic solvent is THF, the second organic solvent is acetone, and the dicarboxylic acid is succinic acid.

48. The process of claim 43, wherein R₁, R₂, R₃, and R₄, are each independently —H, (C₁-C₆)-alkyl, (C₁-C₆)-haloalkyl, (C₂-C₆)-alkenyl, or (C₂-C₆)-alkynyl, halogen, —CF₃, —NO₂, —CN, or —OR₂₅, and R₂₅ is —H; or linear or branched (C₁-C₆)-alkyl, (C₁-C₆)-haloalkyl, (C₂-C₆)-alkenyl, or (C₂-C₆)-alkynyl.

49. The process of claim 43, wherein R₁, R₂, R₃, and R₄, are each independently —H, (C₁-C₆)-alkyl, halogen, —CF₃, or —OR₂₅, and R₂₅ is —H; or linear or branched (C₁-C₆)-alkyl, (C₁-C₆)-haloalkyl, (C₂-C₆)-alkenyl, or (C₂-C₆)-alkynyl.

50. The process of claim 43, wherein R₁ and R₃ are —H, or —OR₂₅, and R₂₅ is —H or (C₁-C₆)-alkyl.

51. The process of claim 50, wherein R₂ is —H or halogen and R₄ is —H or —CF₃.

52. The process of claim 43, wherein the amount of each solvent is less than 0.2 w %.

53. The process of claim 43, wherein the amount of each solvent is less than 0.15 w %.

54. The process of claim 43, wherein the amount of each solvent is less than 0.10 w %.

55. The process of claim 43, wherein the amount of each solvent is less than 0.05 w %.

56. The process of claim 43, wherein the amount of each solvent is less than 0.025 w %.

57. The process of claim 43, wherein the amount of each solvent is less than 0.02 w %.

58. The process of claim 43, wherein the amount of each solvent is less than 0.015 w %.

59. The process of claim 43, wherein the amount of each solvent is less than 0.01 w %.
